(19) United States
(12) Patent Application Publication
(10) Pub. No.: US 2022/0332700 A1
(43) Pub. Date: Oct. 20, 2022

(54) NOVEL APOPTOSIS SIGNAL-REGULATING KINASE 1 INHIBITORS

(71) Applicant: MANKIND PHARMA LTD, New Delhi (IN)

(72) Inventors: Santosh Kumar RAI, Haryana (IN); Rakesh Iswar PATIL, Haryana (IN); Somnath Singha ROY, Haryana (IN); Kalicharan SHARMA, Haryana (IN); Rahul DWIVEDI, Haryana (IN); Srinivasa Reddy BAPURAM, Haryana (IN); Anil KUMAR, Haryana (IN)

(73) Assignee: MANKIND PHARMA LTD, New Delhi (IN)

(21) Appl. No.: 17/620,944

(22) PCT Filed: Jun. 24, 2020
(86) PCT No.: PCT/IN2020/050549

§ 371 Date: Dec. 20, 2021

(30) Foreign Application Priority Data
Jun. 26, 2019 (IN) 201911025419

(51) Int. Cl. C07D 401/14; C07D 413/14; C07D 417/14; C07D 405/14; C07D 491/107; C07D 409/14

(52) U.S. Cl. CPC C07D 401/14; C07D 413/14; C07D 417/14; C07D 405/14; C07D 491/107; C07D 409/14

(57) ABSTRACT

The present invention relates to inhibitors of apoptosis signal-regulating kinase 1 (“ASK1”), a process for synthesis of the compounds of the present invention, composition comprising the compounds and use of the compounds for inhibition of ASK1.

### FIELD OF THE INVENTION

The invention relates to inhibitors of apoptosis signal-regulating kinase 1 (“ASK1”), a process for synthesis of the compounds of the present invention, composition comprising the compounds and use of the compounds for inhibition of ASK1.

### BACKGROUND OF THE INVENTION

Apoptosis signal-regulating kinase 1 (ASK1), is a member of the mitogen-activated protein kinases (MAPKs) family, which are members of the serine/threonine kinase family. ASK1 is also known as mitogen-activated protein kinase kinasekinase 5 (MAPKKK5, MAP3K5), MAP/ERK kinase kinase 5 (MEKK5), MEK kinase 5, MEKK5, MAP/ERK kinase kinase 5.

ASK1 is a member of the large mitogen-activated protein kinase kinase kinase (“MAP3K”) family. ASK1 activation and signaling are associated with broad range of diseases. Compounds that inhibit ASK1 are desired for use in the treatment of ASK1 mediated conditions.

Compounds that inhibit ASK1 are desired for use in the treatment of Nonalcoholic steatohepatitis (NASH). Nonalcoholic steatohepatitis is a liver disease with an etiological constellation characterized by macrovesicular hepatic steatosis, inflammation hepatocyte ballooning and fibrosis. Currently, there is no approved pharmaceutical medicament specifically for the treatment of nonalcoholic steatohepatitis. There is a need for pharmaceutical medicaments to offer additional treatment options for patients suffering from nonalcoholic steatohepatitis.

Non-alcoholic fatty liver disease (NAFLD), comprising several liver diseases including NAFL and NASH, which is the most frequent liver disease world-wide, is a clinical manifestation of overweight and metabolic syndrome. NAFL is a chronic disease that can last several decades and is characterized by predominant macrovesicular steatosis of the liver. The prevalence of NAFL is increasing globally. NASH is an acronym that stands for Non-Alcoholic SteatoHepatitis. It is the most severe form of NAFLD and is characterized by the presence of an abnormal accumulation of fat in the liver which in some individuals can progress to liver cell injury (hepatocellular ballooning) and inflammation. A significant number of NAFL patients develop non-alcoholic steatohepatitis (NASH), fibrosis and subsequently hepatocellular carcinoma (HCC). Hepatocellular ballooning and inflammation—sometimes called necroinflammation—are commonly considered as the drivers of disease progression, or as the underlying causes of the disease. As NASH evolves, over time it can result in excessive scarring in the liver (fibrosis), a natural response to injury which can lead to liver cirrhosis or liver cancer.

Clearly there remains a significant unmet need for novel therapeutic agents as to target NASH progression into cirrhosis and HCC. The object of the present invention is therefore to provide novel treatment agents to this end.

### OBJECT OF THE INVENTION

An object of the invention is to provide compounds as inhibitors of ASK1, a process for preparation of the inhibitors, a composition containing the compounds and utility of the compounds.

### SUMMARY OF THE INVENTION

The present invention discloses novel compounds of 1-(6-(4-isopropyl-4H-1,2,4-triazol-3-yl)pyridin-2-yl)imidazolidin-4-one their pharmaceutically acceptable salts and isomers of formula I:

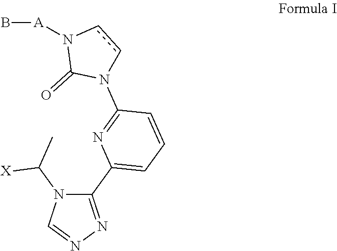

Wherein;
indicates an optional bond

A is absent, H, C1-C₆ alkyl, 5 membered or a 6 membered saturated or unsaturated carbocycle or heterocycle that may be aromatic or aliphatic, which may contain single ring or fused ring and the ring may comprise 1 to 3 heteroatom selected from the group comprising O, N, S, which may be further substituted with R₁, R₁R₂ OH, CH₃, F or Cl; —OR₁, CO, —NR₁R₂, —SO₂(CH₂)n, —CHOHR₁, —CONR₁R₂, —COR₁, —CN, —CF₃; SO₂NR₁R₂, SO₂R₁, O(CH₂)nSO₂CH₃, NH(CR₁R₂)n, NH(CH₂)nR₁, CH₂SO₂R₁, a 3 to 6 membered carbocycle, a 5 or 6 membered heterocycle containing 1 or 2 heteroatoms selected from O, N or S; the heteroatom may be oxidised;

B is absent, H, SO₂, SO₂(CH₂)n; SO₂R₁, SO₂(CH₂)nOR₁, SO₂NHR₁, NH₂(CH₂)n, CO NH₂(CH₂)n, NH₂(CH₂)n, NH₂(CH₂)nR₁CONH₂, NH₂(CH₂)nCN, NHCHR₁CN, HR₁, SO₂Me, 3 to 8 carbocyclic ring optionally substituted by R1 or R2 or R3, 3 to 8 membered heterocyclic ring containing 1 to 3 heteroatoms selected from the group comprising O, N, S which may be optionally substituted by R1 or R2 or R3;

Wherein (CH₂)n can form a cyclic structure depending upon the number of carbon atoms;

Wherein A may be connected to the ring though R1 and B may be connected to A through R1;

X is H, CH₃, (CH₂)n, C₁-C₆ straight or branched or cyclic alkyl, CN, (CH₂)CN, OH, (CH₂)nOH, CF₃;

R1, R2 and R3 are independently selected from the group comprising H, halo, CN, CF₃, CH₃, hydroxyl, C₁-C₆ alkyl, C₁ to C6 alkoxy , C₁-C₆alkoxyalkyl and C₁-C₆cyanoalkyl, amino, SO₂, SO₂C₁-C₆ Alkyl, CH₂CF₃, —(CH₂)nCN, —CH₂OMe, —CH₂SO₂Me, NR₄R₅, CONR₄R₅, C₁-C₆ straight or branched or cyclic alkyl, C₂-C₆ straight or branched alkenyl, C₂-C₆ straight or branched alkynyl, halo-C₁-C₆ alkyl, C₁-C₆ alkyloxy; C₁-C₆ alkylamino, heteroalkyl, C₃-C₈ cycloalkyl, C₃-C₈cycloalkenyl, C₃-C₈heterocycloalkyl, C₃-C₈heterocycloalkenyl, carbocycyl, aryl, and heteroaryl; oxidised heteroaryl, oxidise heterocyclyl, heteroaryl or heterocyclyl that may be optionally substituted with R4 and R5;

R4 and R5 may be selected from H, C₁-C₆ alkyl and C₃-C₈ cycloalkyl, NH₂, NH, C₁-C₆ alkyl n is 0 to 3.

### DETAILED DESCRIPTION OF THE INVENTION

The present invention discloses compounds of formula (I), their derivatives, analogs, tautomeric forms, stereoisomers, polymorphs, hydrates, solvates, pharmaceutically acceptable salts and compositions, metabolites and prodrugs thereof, for intervention in ASK 1 and for their use in treating liver diseases such as non-alcoholic fatty liver disease (NAFLD) and non-alcoholic steatohepatitis (NASH).

The present invention discloses novel compounds of 1-(6-(4-isopropyl-4H-1,2,4-triazol-3-yl)pyridin-2-yl)imidazolidin-4-one their pharmaceutically acceptable salts and isomers of formula I:

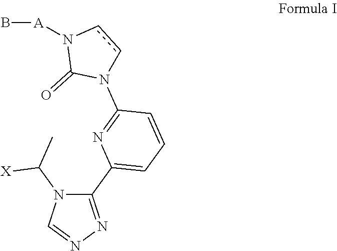

Wherein;
indicates an optional bond

A is absent, H, C1-C₆ alkyl, 5 membered or a 6 membered saturated or unsaturated carbocycle or heterocycle that may be aromatic or aliphatic, which may contain single ring or fused ring and the ring may comprise 1 to 3 heteroatom selected from the group comprising O, N, S, which may be further substituted with R₁, R₁R₂ OH, CH₃, F or Cl; —OR₁, CO, —NR₁R₂, —SO₂(CH₂)n, —CHOHR₁, —CONR₁R₂, —COR₁, —CN, —CF₃; SO₂NR₁R₂, SO₂R₁, O(CH₂)nSO₂CH₃, NH(CR₁R₂)n, NH(CH₂)nR₁, CH₂SO₂R₁, a 3 to 6 membered carbocycle, a 5 or 6 membered heterocycle containing 1 or 2 heteroatoms selected from O, N or S; the heteroatom may be oxidised;

B is absent, H, SO₂, SO₂(CH₂)n; SO₂R₁, SO₂(CH₂)nOR₁, SO₂NHR₁, NH₂(CH₂)n, CO NH₂(CH₂)n, NH₂(CH₂)n, NH₂(CH₂)nR₁CONH₂, NH₂(CH₂)nCN, NHCHR₁CN, NHR₁, SO₂Me, 3 to 8 carbocyclic ring optionally substituted by R1 or R2 or R3, 3 to 8 membered heterocyclic ring containing 1 to 3 heteroatoms selected from the group comprising O, N, S which may be optionally substituted by R1 or R2 or R3;

Wherein (CH₂)n can form a cyclic structure depending upon the number of carbon atoms;

Wherein A may be connected to the ring though R1 and B may be connected to A through R1;

X is H, CH₃, (CH₂)n, C₁-C₆ straight or branched or cyclic alkyl, CN, (CH₂)CN, OH, (CH₂)nOH, CF₃;

R1, R2 and R3 are independently selected from the group comprising H, halo, CN, CF₃, CH₃, hydroxyl, C₁-C₆ alkyl, C₁ to C₆ alkoxy , C₁-C₆alkoxyalkyl and C₁-C₆cyanoalkyl, amino, SO₂, SO₂C₁-C₆ Alkyl, CH₂CF₃, —(CH₂)nCN, —CH₂OMe, —CH₂SO₂Me, NR₄R₅, CONR₄R₅, C₁-C₆ straight or branched or cyclic alkyl, C₂-C₆ straight or branched alkenyl, C₂-C₆ straight or branched alkynyl, halo-C₁-C₆ alkyl, C₁-C₆ alkyloxy; C₁-C₆ alkylamino, heteroalkyl, C₃-C₈cycloalkyl, C₃-C₈cycloalkenyl, C₃-C₈heterocycloalkyl, C₃-C₈heterocycloalkenyl, carbocycyl, aryl, and heteroaryl; oxidised heteroaryl, oxidise heterocyclyl, heteroaryl or heterocyclyl that may be optionally substituted with R4 and R5;

R4 and R5 may be selected from H, C₁-C₆ alkyl and C₃-C₈ cycloalkyl, NH₂, NH, C₁-C₆ alkyl n is 0 to 3.

It is understood by a person skilled in the art that H may be added or removed as per the principles of chemistry.

The compounds disclosed herein and their pharmaceutically acceptable salts can exist as single stereoisomers, racemates, and as mixtures of enantiomers and diastereomers. The compounds disclosed herein can also exist as geometric isomers. All such single stereoisomers, racemates and mixtures thereof, and geometric isomers are intended to be within the scope of the compounds disclosed herein.

Exemplary compounds of the present invention of Formula I are illustrated herein below at Table 1.

TABLE 1
Exemplary compounds of the present invention.

| S. No | Structure | Name |
|---|---|---|
| 1001. | 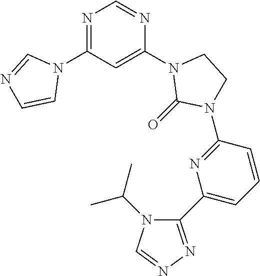 [SMILES: CC(C)n1cnnc1-c1cccc(N2CCN(c3cc(-n4ccnc4)ncn3)C2=O)n1] | 1-(6-(1H-imidazol-1-yl)pyrimidin-4-yl)-3-(6- (4-isopropyl-4H-1,2,4-triazol-3-yl)pyridin-2- yl)imidazolidin-2-one |
| 1002. | 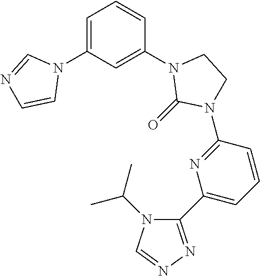 [SMILES: CC(C)n1cnnc1-c1cccc(N2CCN(c3cccc(-n4ccnc4)c3)C2=O)n1] | 1-(3-(1H-imidazol-1-yl)phenyl)-3-(6-(4- isopropyl-4H-1,2,4-triazol-3-yl)pyridin-2- yl)imidazolidin-2-one |
| 1003. | 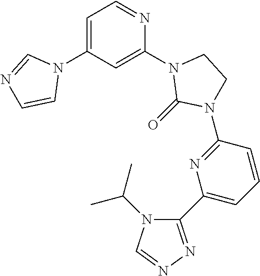 [SMILES: CC(C)n1cnnc1-c1cccc(N2CCN(c3cc(-n4ccnc4)ccn3)C2=O)n1] | 1-(4-(1H-imidazol-1-yl)pyridin-2-yl)-3-(6-(4- isopropyl-4H-1,2,4-triazol-3-yl)pyridin-2- yl)imidazolidin-2-one |
| 1004. | 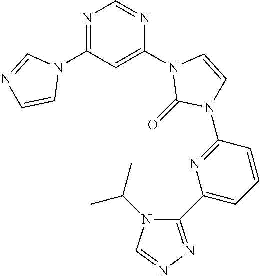 [SMILES: CC(C)n1cnnc1-c1cccc(-n2ccn(-c3cc(-n4ccnc4)ncn3)c2=O)n1] | 1-(6-(1H-imidazol-1-yl)pyrimidin-4-yl)-3-(6- (4-isopropyl-4H-1,2,4-triazol-3-yl)pyridin-2- yl)-1H-imidazol-2(3H)-one |
| 1005. | 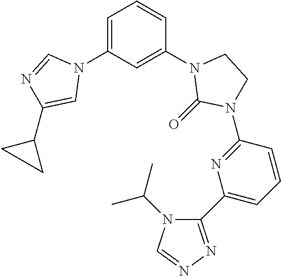 [SMILES: CC(C)n1cnnc1-c1cccc(N2CCN(c3cccc(-n4cnc(C5CC5)c4)c3)C2=O)n1] | 1-(3-(4-cyclopropyl-1H-imidazol-1-yl)phenyl)- 3-(6-(4-isopropyl-4H-1,2,4-triazol-3- yl)pyridin-2-yl)imidazolidin-2-one |
| 1006. | 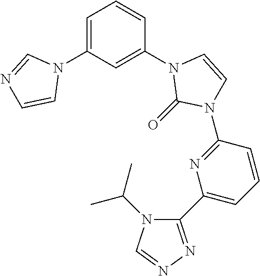 [SMILES: CC(C)n1cnnc1-c1cccc(-n2ccn(-c3cccc(-n4ccnc4)c3)c2=O)n1] | 1-(3-(1H-imidazol-1-yl)phenyl)-3-(6-(4- isopropyl-4H-1,2,4-triazol-3-yl)pyridin-2-yl)- 1H-imidazol-2(3H)-one |
| 1007. | 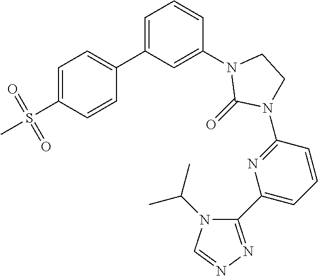 [SMILES: CC(C)n1cnnc1-c1cccc(N2CCN(c3cccc(-c4ccc(S(C)(=O)=O)cc4)c3)C2=O)n1] | 1-(6-(4-isopropyl-4H-1,2,4-triazol-3- yl)pyridin-2-yl)-3-(4′-(methylsulfonyl)-[1,1′- biphenyl]-3-yl)imidazolidin-2-one |
| 1008. | 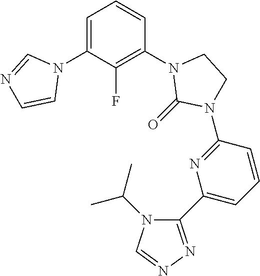 [SMILES: CC(C)n1cnnc1-c1cccc(N2CCN(c3cccc(-n4ccnc4)c3F)C2=O)n1] | 1-(2-fluoro-3-(1H-imidazol-1-yl)phenyl)-3-(6- 4-isopropyl-4H-1,2,4-triazol-3-yl)pyridin-2- yl)imidazolidin-2-one |
| 1009. | 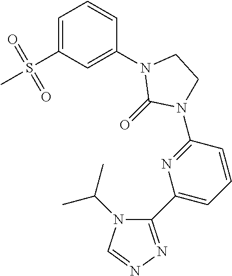 [SMILES: CC(C)n1cnnc1-c1cccc(N2CCN(c3cccc(S(C)(=O)=O)c3)C2=O)n1] | 1-(6-(4-isopropyl-4H-1,2,4-triazol-3- yl)pyridin-2-yl)-3-(3- (methylsulfonyl)phenyl)imidazolidin-2-one |
| 1010. | 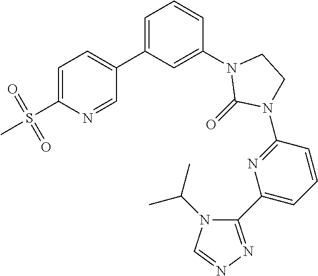 [SMILES: CC(C)n1cnnc1-c1cccc(N2CCN(c3cccc(-c4ccc(S(C)(=O)=O)nc4)c3)C2=O)n1] | 1-(6-(4-isopropyl-4H-1,2,4-triazol-3- yl)pyridin-2-yl)-3-(3-(6- (methylsulfonyl)pyridin-3- yl)phenyl)imidazolidin-2-one |
| 1011. | 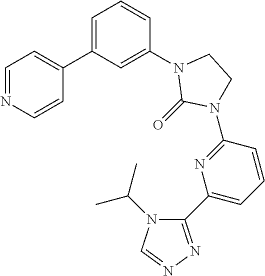 [SMILES: CC(C)n1cnnc1-c1cccc(N2CCN(c3cccc(-c4ccncc4)c3)C2=O)n1] | (1-(6-(4-isopropyl-4H-1,2,4-triazol-3- yl)pyridin-2-yl)-3-(3-(pyridin-4- yl)phenyl)imidazolidin-2-one |
| 1012. | 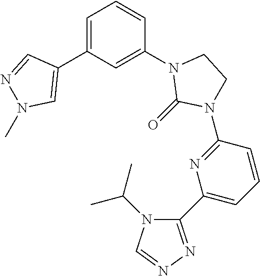 [SMILES: CC(C)n1cnnc1-c1cccc(N2CCN(c3cccc(-c4cnn(C)c4)c3)C2=O)n1] | 1-(6-(4-isopropyl-4H-1,2,4-triazol-3- yl)pyridin-2-yl)-3-(3-(1-methyl-1H-pyrazol-4- yl)phenyl)imidazolidin-2-one |
| 1013. | 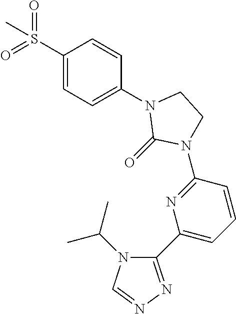 [SMILES: CC(C)n1cnnc1-c1cccc(N2CCN(c3ccc(S(C)(=O)=O)cc3)C2=O)n1] | 1-(6-(4-isopropyl-4H-1,2,4-triazol-3- yl)pyridin-2-yl)-3-(4- (methylsulfonyl)phenyl)imidazolidin-2-one |
| 1014. | 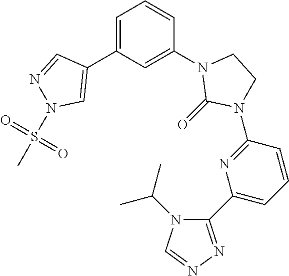 [SMILES: CC(C)n1cnnc1-c1cccc(N2CCN(c3cccc(-c4cnn(S(C)(=O)=O)c4)c3)C2=O)n1] | 1-(6-(4-isopropyl-4H-1,2,4-triazol-3- yl)pyridin-2-yl)-3-(3-(1-(methylsulfonyl)-1H- pyrazol-4-yl)phenyl)imidazolidin-2-one |
| 1015. | 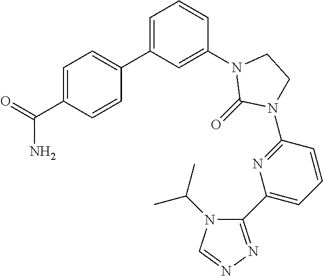 [SMILES: CC(C)n1cnnc1-c1cccc(N2CCN(c3cccc(-c4ccc(C(N)=O)cc4)c3)C2=O)n1] | 3′-(3-(6-(4-isopropyl-4H-1,2,4-triazol-3- yl)pyridin-2-yl)-2-oxoimidazolidin-1-yl)-[1,1′- biphenyl]-4-carboxamide |
| 1016. | 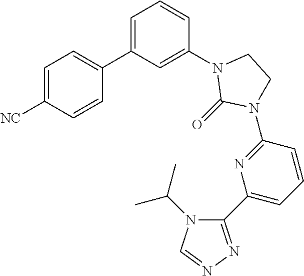 [SMILES: CC(C)n1cnnc1-c1cccc(N2CCN(c3cccc(-c4ccc(C#N)cc4)c3)C2=O)n1] | 3′-(3-(6-(4-isopropyl-4H-1,2,4-triazol-3- yl)pyridin-2-yl)-2-oxoimidazolidin-1-yl)-[1,1′- biphenyl]-4-carbonitrile |
| 1017. | 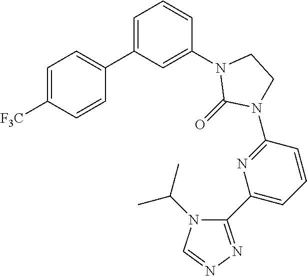 [SMILES: CC(C)n1cnnc1-c1cccc(N2CCN(c3cccc(-c4ccc(C(F)(F)F)cc4)c3)C2=O)n1] | 1-(6-(4-isopropyl-4H-1,2,4-triazol-3- yl)pyridin-2-yl)-3-(4′-(trifluoromethyl)-[1,1′- biphenyl]-3-yl)imidazolidin-2-one |
| 1018. | 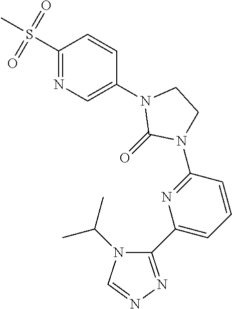 [SMILES: CC(C)n1cnnc1-c1cccc(N2CCN(c3ccc(S(C)(=O)=O)nc3)C2=O)n1] | 1-(6-(4-isopropyl-4H-1,2,4-triazol-3- yl)pyridin-2-yl)-3-(6-(methylsulfonyl)pyridin- 3-yl)imidazolidin-2-one |
| 1019. | 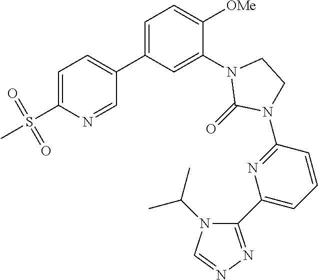 [SMILES: COc1ccc(-c2ccc(S(C)(=O)=O)nc2)cc1N1CCN(c2cccc(-c3nncn3C(C)C)n2)C1=O] | 1-(6-(4-isopropyl-4H-1,2,4-triazol-3- yl)pyridin-2-yl)-3-(2-methoxy-5-(6- (methylsulfonyl)pyridin-3- yl)phenyl)imidazolidin-2-one |
| 1020. | 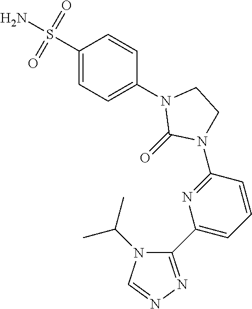 [SMILES: CC(C)n1cnnc1-c1cccc(N2CCN(c3ccc(S(N)(=O)=O)cc3)C2=O)n1] | 4-(3-(6-(4-isopropyl-4H-1,2,4-triazol-3- yl)pyridin-2-yl)-2-oxoimidazolidin-1- yl)benzenesulfonamide |
| 1021. | 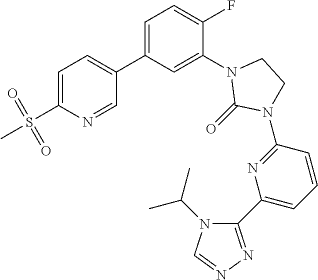 [SMILES: CC(C)n1cnnc1-c1cccc(N2CCN(c3cc(-c4ccc(S(C)(=O)=O)nc4)ccc3F)C2=O)n1] | 1-(2-fluoro-5-(6-(methylsulfonyl)pyridin-3- yl)phenyl)-3-(6-(4-isopropyl-4H-1,2,4-triazol- 3-yl)pyridin-2-yl)imidazolidin-2-one |
| 1022. | 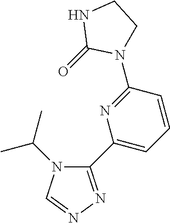 [SMILES: CC(C)n1cnnc1-c1cccc(N2CCNC2=O)n1] | 1-(6-(4-isopropyl-4H-1,2,4-triazol-3- yl)pyridin-2-yl)imidazolidin-2-one |
| 1023. | 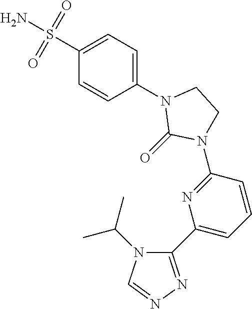 [SMILES: CC(C)n1cnnc1-c1cccc(N2CCN(c3ccc(S(N)(=O)=O)cc3)C2=O)n1] | 4-(3-(6-(4-isopropyl-4H-1,2,4-triazol-3- yl)pyridin-2-yl)-2-oxoimidazolidin-1- yl)benzenesulfonamide |
| 1024. | 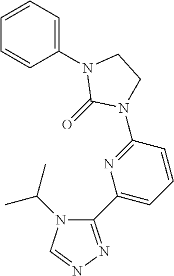 [SMILES: CC(C)n1cnnc1-c1cccc(N2CCN(c3ccccc3)C2=O)n1] | 1-(6-(4-isopropyl-4H-1,2,4-triazol-3- yl)pyridin-2-yl)-3-phenylimidazolidin-2-one |
| 1025. | 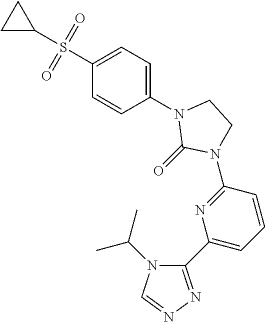 [SMILES: CC(C)n1cnnc1-c1cccc(N2CCN(c3ccc(S(=O)(=O)C4CC4)cc3)C2=O)n1] | 1-(4-(cyclopropylsulfonyl)phenyl)-3-(6-(4- isopropyl-4H-1,2,4-triazol-3-yl)pyridin-2- yl)imidazolidin-2-one |
| 1026. | 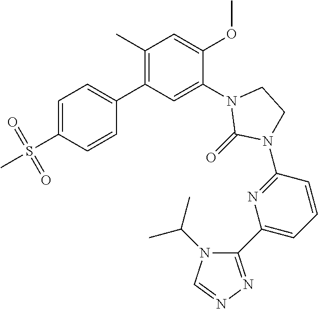 [SMILES: COc1cc(C)c(-c2ccc(S(C)(=O)=O)cc2)cc1N1CCN(c2cccc(-c3nncn3C(C)C)n2)C1=O] | 1-(6-(4-isopropyl-4H-1,2,4-triazol-3- yl)pyridin-2-yl)-3-(4-methoxy-6-methyl-4′- (methylsulfonyl)-[1,1′-biphenyl]-3- yl)imidazolidin-2-one |
| 1027. | 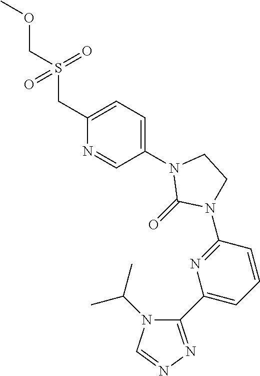 [SMILES: COCS(=O)(=O)Cc1ccc(N2CCN(c3cccc(-c4nncn4C(C)C)n3)C2=O)cn1] | 1-(6-(2-methoxyethylsulfonyl)pyridin-3-yl)-3- (6-(4-isopropyl-4H-1,2,4-triazol-3-yl)pyridin- 2-yl)imidazolidin-2-one |
| 1028. | 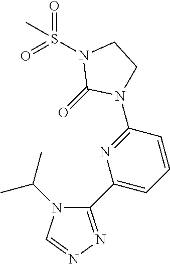 [SMILES: CC(C)n1cnnc1-c1cccc(N2CCN(S(C)(=O)=O)C2=O)n1] | 1-(6-(4-isopropyl-4H-1,2,4-triazol-3- yl)pyridin-2-yl)-3- (methylsulfonyl)imidazolidin-2-one |
| 1029. | 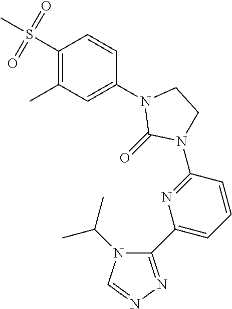 [SMILES: Cc1cc(N2CCN(c3cccc(-c4nncn4C(C)C)n3)C2=O)ccc1S(C)(=O)=O] | 1-(6-(4-isopropyl-4H-1,2,4-triazol-3- yl)pyridin-2-yl)-3-(3-methyl-4- (methylsulfonyl)phenyl)imidazolidin-2-one |
| 1030. | 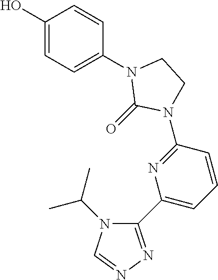 [SMILES: CC(C)n1cnnc1-c1cccc(N2CCN(c3ccc(O)cc3)C2=O)n1] | 1-(4-hydroxyphenyl)-3-(6-(4-isopropyl-4H- 1,2,4-triazol-3-yl)pyridin-2-yl)imidazolidin-2- one |
| 1031. | 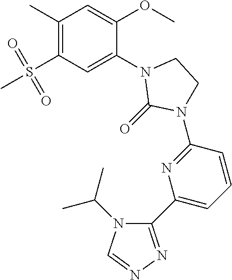 [SMILES: COc1cc(C)c(S(C)(=O)=O)cc1N1CCN(c2cccc(-c3nncn3C(C)C)n2)C1=O] | 1-(6-(4-isopropyl-4H-1,2,4-triazol-3- yl)pyridin-2-yl)-3-(2-methoxy-4-methyl-5- (methylsulfonyl)phenyl)imidazolidin-2-one |
| 1032. | 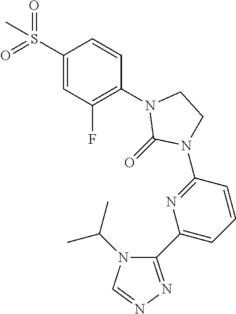 [SMILES: CC(C)n1cnnc1-c1cccc(N2CCN(c3ccc(S(C)(=O)=O)cc3F)C2=O)n1] | 1-(2-fluoro-4-(methylsulfonyl)phenyl)-3-(6-(4- isopropyl-4H-1,2,4-triazol-3-yl)pyridin-2- yl)imidazolidin-2-one |
| 1033. | 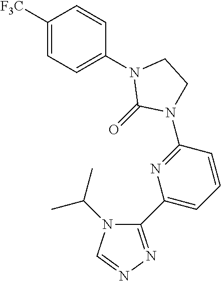 [SMILES: CC(C)n1cnnc1-c1cccc(N2CCN(c3ccc(C(F)(F)F)cc3)C2=O)n1] | 1-(4-(trifluoromethyl)phenyl)-3-(6-(4- isopropyl-4H-1,2,4-triazol-3-yl)pyridin-2- yl)imidazolidin-2-one |
| 1034. | 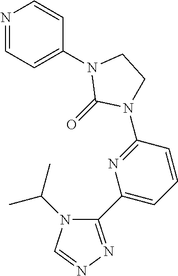 [SMILES: CC(C)n1cnnc1-c1cccc(N2CCN(c3ccncc3)C2=O)n1] | 1-(6-(4-isopropyl-4H-1,2,4-triazol-3- yl)pyridin-2-yl)-3-(pyridin-4-yl)imidazolidin- 2-one |
| 1035. | 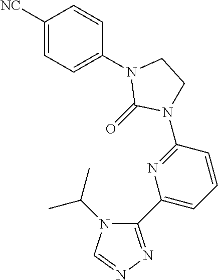 [SMILES: CC(C)n1cnnc1-c1cccc(N2CCN(c3ccc(C#N)cc3)C2=O)n1] | 4-(3-(6-(4-isopropyl-4H-1,2,4-triazol-3- yl)pyridin-2-yl)-2-oxoimidazolidin-1- yl)benzonitrile |
| 1036. | 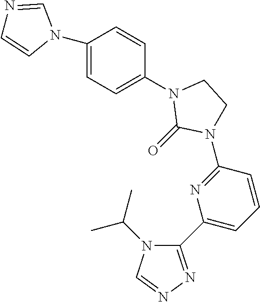 [SMILES: CC(C)n1cnnc1-c1cccc(N2CCN(c3ccc(-n4ccnc4)cc3)C2=O)n1] | 1-(4-(1H-imidazol-1-yl)phenyl)-3-(6-(4- isopropyl-4H-1,2,4-triazol-3-yl)pyridin-2- yl)imidazolidin-2-one |
| 1037. | 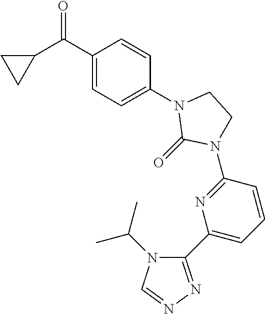 [SMILES: CC(C)n1cnnc1-c1cccc(N2CCN(c3ccc(C(=O)C4CC4)cc3)C2=O)n1] | 1-(4-(cyclopropanecarbonyl)phenyl)-3-(6-(4- isopropyl-4H-1,2,4-triazol-3-yl)pyridin-2- yl)imidazolidin-2-one |
| 1038. | 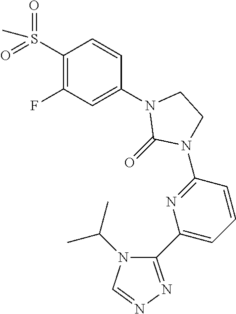 [SMILES: CC(C)n1cnnc1-c1cccc(N2CCN(c3ccc(S(C)(=O)=O)c(F)c3)C2=O)n1] | 1-(3-fluoro-4-(methylsulfonyl)phenyl)-3-(6-(4- isopropyl-4H-1,2,4-triazol-3-yl)pyridin-2- yl)imidazolidin-2-one |
| 1039. | 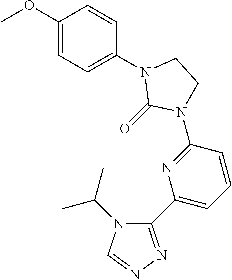 [SMILES: COc1ccc(N2CCN(c3cccc(-c4nncn4C(C)C)n3)C2=O)cc1] | 1-(6-(4-isopropyl-4H-1,2,4-triazol-3- yl)pyridin-2-yl)-3-(4- methoxyphenyl)imidazolidin-2-one |
| 1040. | 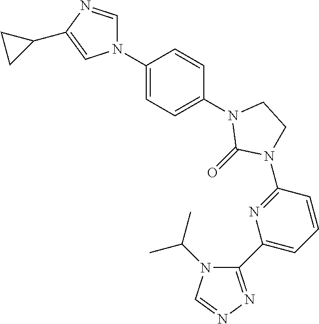 [SMILES: CC(C)n1cnnc1-c1cccc(N2CCN(c3ccc(-n4cnc(C5CC5)c4)cc3)C2=O)n1] | 1-(4-(4-cyclopropyl-1H-imidazol-1-yl)phenyl)- 3-(6-(4-isopropyl-4H-1,2,4-triazol-3- yl)pyridin-2-yl)imidazolidin-2-one |
| 1041. | 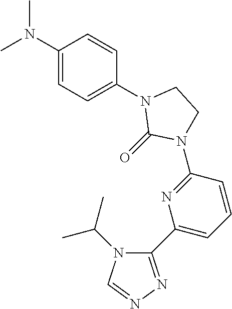 [SMILES: CC(C)n1cnnc1-c1cccc(N2CCN(c3ccc(N(C)C)cc3)C2=O)n1] | 1-(4-(dimethylamino)phenyl)-3-(6-(4- isopropyl-4H-1,2,4-triazol-3-yl)pyridin-2- yl)imidazolidin-2-one |
| 1042. | 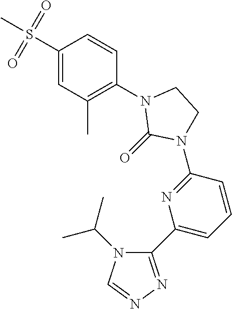 [SMILES: Cc1cc(S(C)(=O)=O)ccc1N1CCN(c2cccc(-c3nncn3C(C)C)n2)C1=O] | 1-(6-(4-isopropyl-4H-1,2,4-triazol-3- yl)pyridin-2-yl)-3-(2-methyl-4- (methylsulfonyl)phenyl)imidazolidin-2-one |
| 1043. | 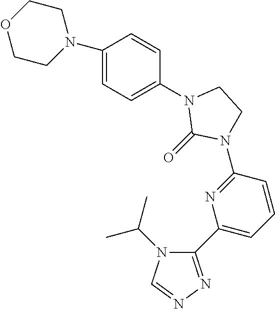 [SMILES: CC(C)n1cnnc1-c1cccc(N2CCN(c3ccc(N4CCOCC4)cc3)C2=O)n1] | 1-(6-(4-isopropyl-4H-1,2,4-triazol-3- yl)pyridin-2-yl)-3-(4- morpholinophenyl)imidazolidin-2-one |
| 1044. | 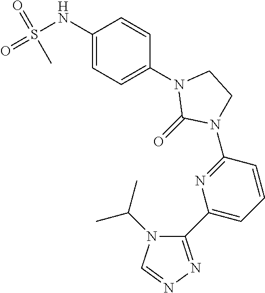 [SMILES: CC(C)n1cnnc1-c1cccc(N2CCN(c3ccc(NS(C)(=O)=O)cc3)C2=O)n1] | N-(4-(3-(6-(4-isopropyl-4H-1,2,4-triazol-3- yl)pyridin-2-yl)-2-oxoimidazolidin-1- yl)phenyl)methanesulfonamide |
| 1045. | 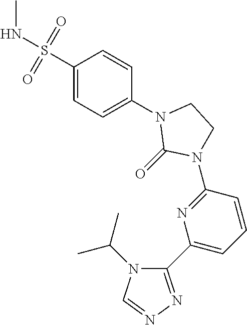 [SMILES: CNS(=O)(=O)c1ccc(N2CCN(c3cccc(-c4nncn4C(C)C)n3)C2=O)cc1] | 4-(3-(6-(4-isopropyl-4H-1,2,4-triazol-3- yl)pyridin-2-yl)-2-oxoimidazolidin-1-yl)-N- methylbenzenesulfonamide |
| 1046. | 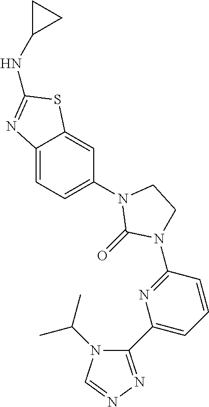 [SMILES: CC(C)n1cnnc1-c1cccc(N2CCN(c3ccc4nc(NC5CC5)sc4c3)C2=O)n1] | N-cyclopropyl-6-(3-(6-(4-isopropyl-4H-1,2,4- triazol-3-yl)pyridin-2-yl)-2-oxoimidazolidin-1- yl)benzo[d]thiazol-2-aminium 2,2,2- trifluoroacetate |
| 1047. | 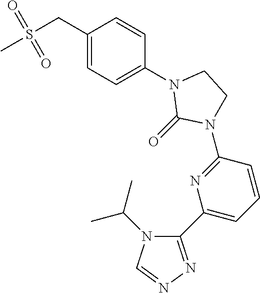 [SMILES: CC(C)n1cnnc1-c1cccc(N2CCN(c3ccc(CS(C)(=O)=O)cc3)C2=O)n1] | 1-(6-(4-isopropyl-4H-1,2,4-triazol-3- yl)pyridin-2-yl)-3-(4- ((methylsulfonyl)methyl)phenyl)imidazolidin- 2-one |
| 1048. | 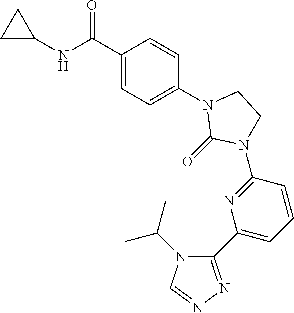 [SMILES: CC(C)n1cnnc1-c1cccc(N2CCN(c3ccc(C(=O)NC4CC4)cc3)C2=O)n1] | N-cyclopropyl-4-(3-(6-(4-isopropyl-4H-1,2,4- triazol-3-yl)pyridin-2-yl)-2-oxoimidazolidin-1- yl)benzamide |
| 1049. | 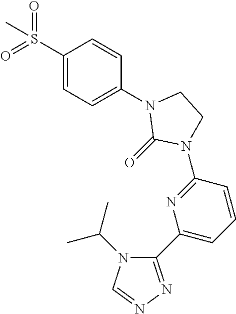 [SMILES: CC(C)n1cnnc1-c1cccc(N2CCN(c3ccc(S(C)(=O)=O)cc3)C2=O)n1] | 1-(6-(4-cyclopropyl-4H-1,2,4-triazol-3- yl)pyridin-2-yl)-3-(4- (methylsulfonyl)phenyl)imidazolidin-2-one |
| 1050. | 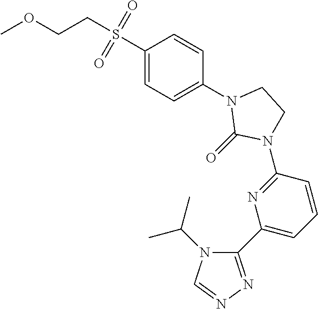 [SMILES: COCCS(=O)(=O)c1ccc(N2CCN(c3cccc(-c4nncn4C(C)C)n3)C2=O)cc1] | 1-(4-(2-methoxyethylsulfonyl)phenyl)-3-(6-(4- isopropyl-4H-1,2,4-triazol-3-yl)pyridin-2- yl)imidazolidin-2-one |
| 1051. | 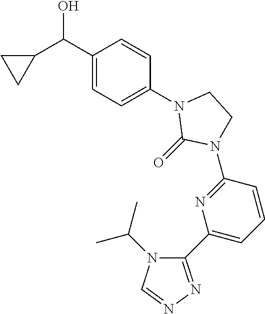 [SMILES: CC(C)n1cnnc1-c1cccc(N2CCN(c3ccc(C(O)C4CC4)cc3)C2=O)n1] | 1-(4-(cyclopropyl(hydroxy)methyl)phenyl)-3- (6-(4-isopropyl-4H-1,2,4-triazol-3-yl)pyridin- 2-yl)imidazolidin-2-one |
| 1052. | 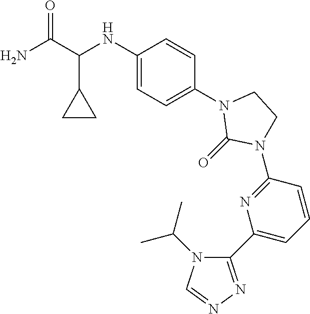 [SMILES: CC(C)n1cnnc1-c1cccc(N2CCN(c3ccc(NC(C(N)=O)C4CC4)cc3)C2=O)n1] | 2-(4-(3-(6-(4-isopropyl-4H-1,2,4-triazol-3- yl)pyridin-2-yl)-2-oxoimidazolidin-1- yl)phenylamino)-2-cyclopropylacetamide |
| 1053. | 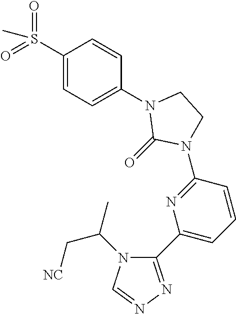 [SMILES: CC(CC#N)n1cnnc1-c1cccc(N2CCN(c3ccc(S(C)(=O)=O)cc3)C2=O)n1] | 3-(3-(6-(3-(4-(methylsulfonyl)phenyl)-2- oxoimidazolidin-1-yl)pyridin-2-yl)-4H-1,2,4- triazol-4-yl)butanenitrile |
| 1054. | 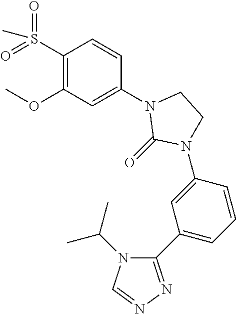 [SMILES: COc1cc(N2CCN(c3cccc(-c4nncn4C(C)C)c3)C2=O)ccc1S(C)(=O)=O] | 1-(6-(4-isopropyl-4H-1,2,4-triazol-3- yl)pyridin-2-yl)-3-(3-methoxy-4- (methylsulfonyl)phenyl)imidazolidin-2-one |
| 1055. | 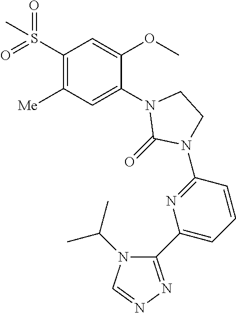 [SMILES: COc1cc(S(C)(=O)=O)c(C)cc1N1CCN(c2cccc(-c3nncn3C(C)C)n2)C1=O] | 1-(6-(4-isopropyl-4H-1,2,4-triazol-3- yl)pyridin-2-yl)-3-(2-methoxy-5-methyl-4- (methylsulfonyl)phenyl)imidazolidin-2-one |
| 1056. | 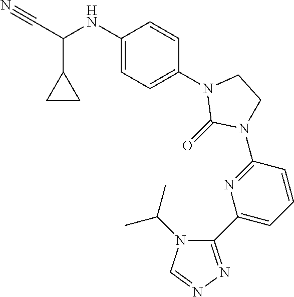 [SMILES: CC(C)n1cnnc1-c1cccc(N2CCN(c3ccc(NC(C#N)C4CC4)cc3)C2=O)n1] | 2-(4-(3-(6-(4-isopropyl-4H-1,2,4-triazol-3- yl)pyridin-2-yl)-2-oxoimidazolidin-1- yl)phenylamino)-2-cyclopropylacetonitrile |
| 1057. | 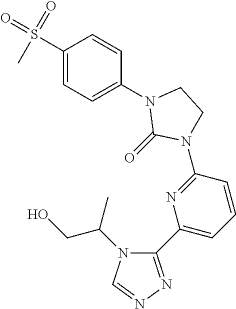 [SMILES: CC(CO)n1cnnc1-c1cccc(N2CCN(c3ccc(S(C)(=O)=O)cc3)C2=O)n1] | 1-(6-(4-(1-hydroxypropan-2-yl)-4H-1,2,4- triazol-3-yl)pyridin-2-yl)-3-(4- (methylsulfonyl)phenyl)imidazolidin-2-one |
| 1058. | 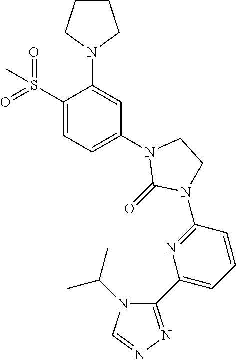 [SMILES: CC(C)n1cnnc1-c1cccc(N2CCN(c3ccc(S(C)(=O)=O)c(N4CCCC4)c3)C2=O)n1] | 1-(6-(4-isopropyl-4H-1,2,4-triazol-3- yl)pyridin-2-yl)-3-(4-(methylsulfonyl)-3- (pyrrolidin-1-yl)phenyl)imidazolidin-2-one |
| 1059. | 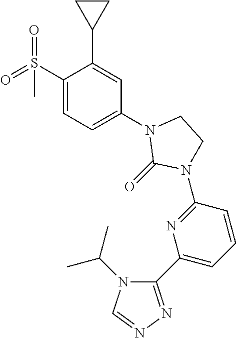 [SMILES: CC(C)n1cnnc1-c1cccc(N2CCN(c3ccc(S(C)(=O)=O)c(C4CC4)c3)C2=O)n1] | 1-(3-cyclopropyl-4-(methylsulfonyl)phenyl)-3- (6-(4-isopropyl-4H-1,2,4-triazol-3-yl)pyridin- 2-yl)imidazolidin-2-one |
| 1060. | 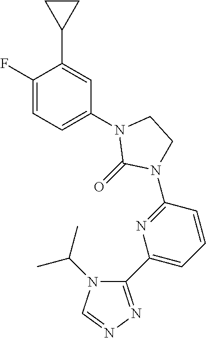 [SMILES: CC(C)n1cnnc1-c1cccc(N2CCN(c3ccc(F)c(C4CC4)c3)C2=O)n1] | 1-(3-cyclopropyl-4-fluorophenyl)-3-(6-(4- isopropyl-4H-1,2,4-triazol-3-yl)pyridin-2- yl)imidazolidin-2-one |
| 1061. | 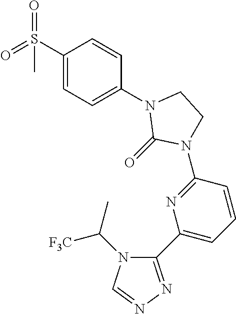 [SMILES: CC(n1cnnc1-c1cccc(N2CCN(c3ccc(S(C)(=O)=O)cc3)C2=O)n1)C(F)(F)F] | 1-(6-(4-(1,1,1-trifluoropropan-2-yl)-4H-1,2,4- triazol-3-yl)pyridin-2-yl)-3-(4- (methylsulfonyl)phenyl)imidazolidin-2-one |
| 1062. | 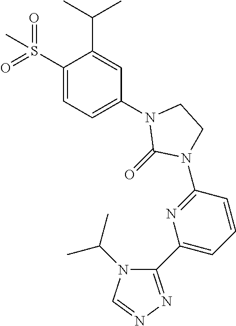 [SMILES: CC(C)c1cc(N2CCN(c3cccc(-c4nncn4C(C)C)n3)C2=O)ccc1S(C)(=O)=O] | 1-(3-isopropyl-4-(methylsulfonyl)phenyl)-3-(6- (4-isopropyl-4H-1,2,4-triazol-3-yl)pyridin-2- yl)imidazolidin-2-one |
| 1063. | 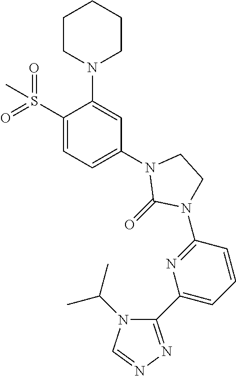 [SMILES: CC(C)n1cnnc1-c1cccc(N2CCN(c3ccc(S(C)(=O)=O)c(N4CCCCC4)c3)C2=O)n1] | 1-(6-(4-isopropyl-4H-1,2,4-triazol-3- yl)pyridin-2-yl)-3-(4-(methylsulfonyl)-3- (piperidin-1-yl)phenyl)imidazolidin-2-one |
| 1064. | 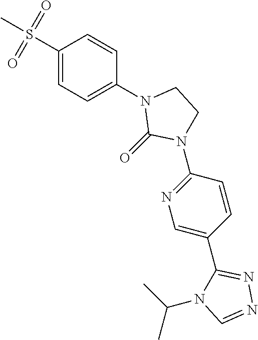 [SMILES: CC(C)n1cnnc1-c1ccc(N2CCN(c3ccc(S(C)(=O)=O)cc3)C2=O)nc1] | 1-(5-(4-isopropyl-4H-1,2,4-triazol-3- yl)pyridin-2-yl)-3-(4- (methylsulfonyl)phenyl)imidazolidin-2-one |
| 1065. | 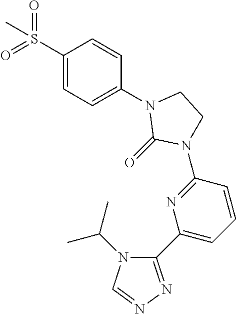 [SMILES: CC(C)n1cnnc1-c1cccc(N2CCN(c3ccc(S(C)(=O)=O)cc3)C2=O)n1] | 1-(6-(4-isopropyl-4H-1,2,4-triazol-3- yl)pyridin-2-yl)-3-(4- (methylsulfonyl)phenyl)imidazolidin-2-one |
| 1066. | 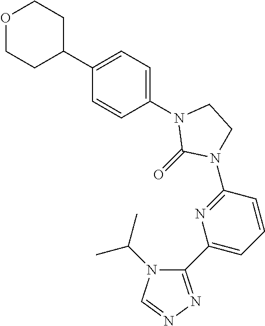 [SMILES: CC(C)n1cnnc1-c1cccc(N2CCN(c3ccc(C4CCOCC4)cc3)C2=O)n1] | 1-(4-(tetrahydro-2H-pyran-4-yl)phenyl)-3-(6- (4-isopropyl-4H-1,2,4-triazol-3-yl)pyridin-2- yl)imidazolidin-2-one |
| 1067. | 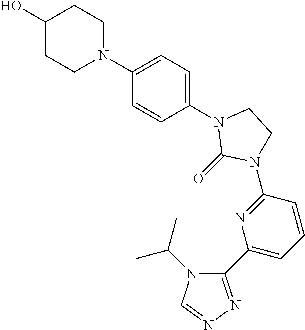 [SMILES: CC(C)n1cnnc1-c1cccc(N2CCN(c3ccc(N4CCC(O)CC4)cc3)C2=O)n1] | 1-(4-(4-hydroxypiperidin-1-yl)phenyl)-3-(6-(4- isopropyl-4H-1,2,4-triazol-3-yl)pyridin-2- yl)imidazolidin-2-one |
| 1068. | 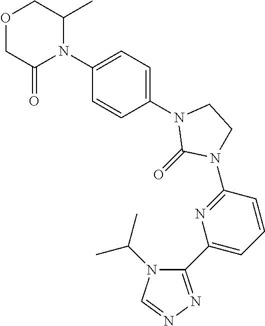 [SMILES: CC1COCC(=O)N1c1ccc(N2CCN(c3cccc(-c4nncn4C(C)C)n3)C2=O)cc1] | 4-(4-(3-(6-(4-isopropyl-4H-1,2,4-triazol-3- yl)pyridin-2-yl)-2-oxoimidazolidin-1- yl)phenyl)-5-methylmorpholin-3-one |
| 1069. | 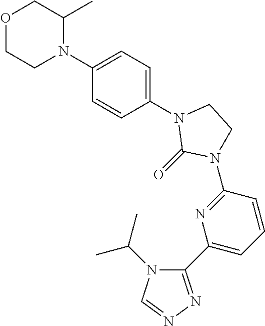 [SMILES: CC1COCCN1c1ccc(N2CCN(c3cccc(-c4nncn4C(C)C)n3)C2=O)cc1] | 1-(6-(4-isopropyl-4H-1,2,4-triazol-3- yl)pyridin-2-yl)-3-(4-(3- methylmorpholino)phenyl)imidazolidin-2-one |
| 1070. | 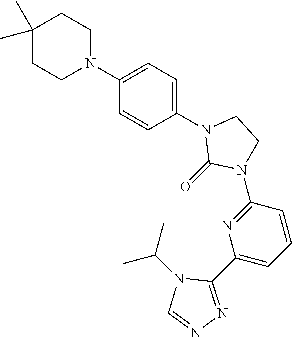 [SMILES: CC(C)n1cnnc1-c1cccc(N2CCN(c3ccc(N4CCC(C)(C)CC4)cc3)C2=O)n1] | 1-(6-(4-isopropyl-4H-1,2,4-triazol-3- yl)pyridin-2-yl)-3-(4-(4,4-dimethylpiperidin-1- yl)phenyl)imidazolidin-2-one |
| 1071. | 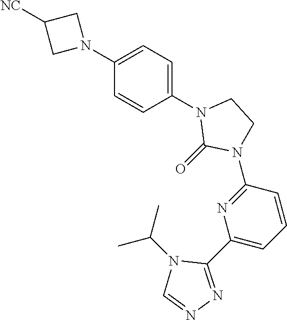 [SMILES: CC(C)n1cnnc1-c1cccc(N2CCN(c3ccc(N4CC(C#N)C4)cc3)C2=O)n1] | 1-(4-(3-(6-(4-isopropyl-4H-1,2,4-triazol-3- yl)pyridin-2-yl)-2-oxoimidazolidin-1- yl)phenyl)azetidine-3-carbonitrile |
| 1072. | 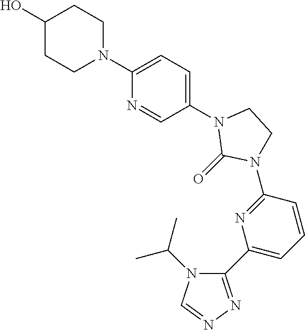 [SMILES: CC(C)n1cnnc1-c1cccc(N2CCN(c3ccc(N4CCC(O)CC4)nc3)C2=O)n1] | 1-(6-(4-hydroxypiperidin-1-yl)pyridin-3-yl)-3- (6-(4-isopropyl-4H-1,2,4-triazol-3-yl)pyridin- 2-yl)imidazolidin-2-one |
| 1073. | 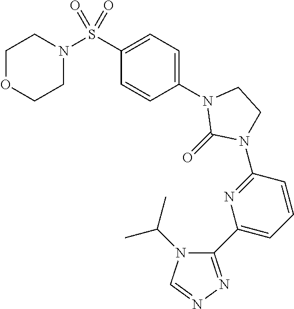 [SMILES: CC(C)n1cnnc1-c1cccc(N2CCN(c3ccc(S(=O)(=O)N4CCOCC4)cc3)C2=O)n1] | 1-(6-(4-isopropyl-4H-1,2,4-triazol-3- yl)pyridin-2-yl)-3-(4- (morpholinosulfonyl)phenyl)imidazolidin-2- one |
| 1074. | 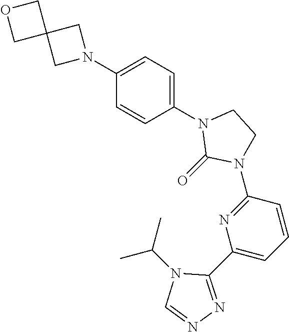 [SMILES: CC(C)n1cnnc1-c1cccc(N2CCN(c3ccc(N4CC5(COC5)C4)cc3)C2=O)n1] | 1-(4-(2-oxa-6-azaspiro[3.3]heptan-6- yl)phenyl)-3-(6-(4-isopropyl-4H-1,2,4-triazol- 3-yl)pyridin-2-yl)imidazolidin-2-one |
| 1075. | 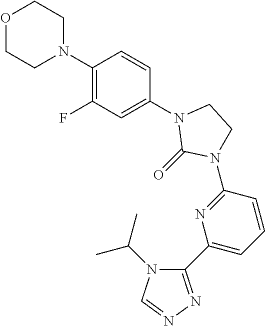 [SMILES: CC(C)n1cnnc1-c1cccc(N2CCN(c3ccc(N4CCOCC4)c(F)c3)C2=O)n1] | 1-(3-fluoro-4-morpholinophenyl)-3-(6-(4- isopropyl-4H-1,2,4-triazol-3-yl)pyridin-2- yl)imidazolidin-2-one |
| 1076. | 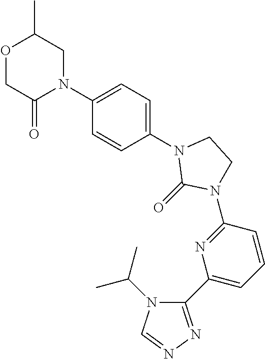 [SMILES: CC1CN(c2ccc(N3CCN(c4cccc(-c5nncn5C(C)C)n4)C3=O)cc2)C(=O)CO1] | 4-(4-(3-(6-(4-isopropyl-4H-1,2,4-triazol-3- yl)pyridin-2-yl)-2-oxoimidazolidin-1- yl)phenyl)-6-methylmorpholin-3-one |
| 1077. | 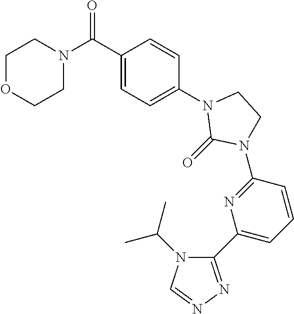 [SMILES: CC(C)n1cnnc1-c1cccc(N2CCN(c3ccc(C(=O)N4CCOCC4)cc3)C2=O)n1] | 1-(6-(4-isopropyl-4H-1,2,4-triazol-3- yl)pyridin-2-yl)-3-(4-(morpholine-4- carbonyl)phenyl)imidazolidin-2-one |
| 1078. | 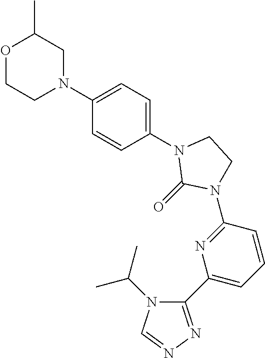 [SMILES: CC1CN(c2ccc(N3CCN(c4cccc(-c5nncn5C(C)C)n4)C3=O)cc2)CCO1] | 1-(6-(4-isopropyl-4H-1,2,4-triazol-3- yl)pyridin-2-yl)-3-(4-(2- methylmorpholino)phenyl)imidazolidin-2-one |
| 1079. | 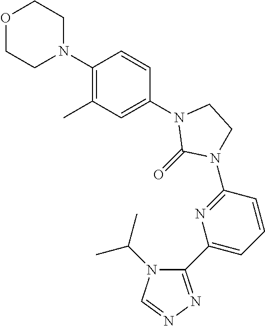 [SMILES: Cc1cc(N2CCN(c3cccc(-c4nncn4C(C)C)n3)C2=O)ccc1N1CCOCC1] | 1-(6-(4-isopropyl-4H-1,2,4-triazol-3- yl)pyridin-2-yl)-3-(3-methyl-4- morpholinophenyl)imidazolidin-2-one |
| 1080. | 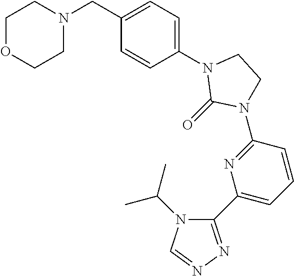 [SMILES: CC(C)n1cnnc1-c1cccc(N2CCN(c3ccc(CN4CCOCC4)cc3)C2=O)n1] | 1-(6-(4-isopropyl-4H-1,2,4-triazol-3- yl)pyridin-2-yl)-3-(4- (morpholinomethyl)phenyl)imidazolidin-2-one |
| 1081. | 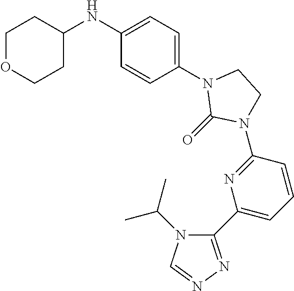 [SMILES: CC(C)n1cnnc1-c1cccc(N2CCN(c3ccc(NC4CCOCC4)cc3)C2=O)n1] | 1-(6-(4-isopropyl-4H-1,2,4-triazol-3- yl)pyridin-2-yl)-3-(4-(tetrahydro-2H-pyran-4- ylamino)phenyl)imidazolidin-2-one |
| 1082. | 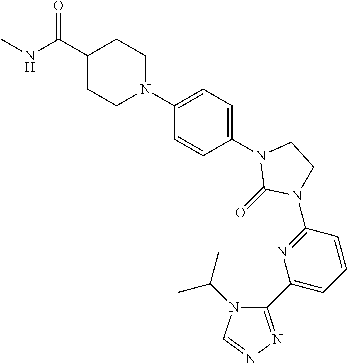 [SMILES: CNC(=O)C1CCN(c2ccc(N3CCN(c4cccc(-c5nncn5C(C)C)n4)C3=O)cc2)CC1] | 1-(4-(3-(6-(4-isopropyl-4H-1,2,4-triazol-3- yl)pyridin-2-yl)-2-oxoimidazolidin-1- yl)phenyl)-N-methylpiperidine-4-carboxamide |
| 1083. | 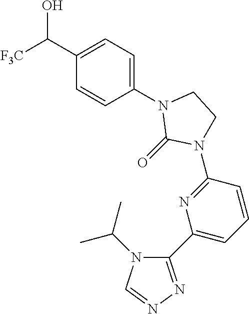 [SMILES: CC(C)n1cnnc1-c1cccc(N2CCN(c3ccc(C(O)C(F)(F)F)cc3)C2=O)n1] | 1-(4-(2,2,2-trifluoro-1-hydroxy ethyl)phenyl)-3- (6-(4-isopropyl-4H-1,2,4-triazol-3-yl)pyridin- 2-yl)imidazolidin-2-one |
| 1084. | 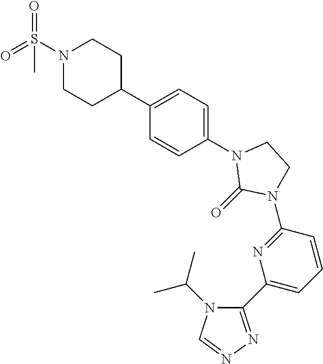 [SMILES: CC(C)n1cnnc1-c1cccc(N2CCN(c3ccc(C4CCN(S(C)(=O)=O)CC4)cc3)C2=O)n1] | 1-(6-(4-isopropyl-4H-1,2,4-triazol-3- yl)pyridin-2-yl)-3-(4-(1- (methylsulfonyl)piperidin-4- yl)phenyl)imidazolidin-2-one |
| 1085. | 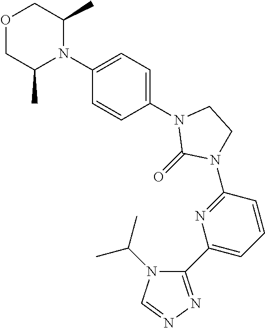 [SMILES: CC(C)n1cnnc1-c1cccc(N2CCN(c3ccc(N4[C@H](C)COC[C@@H]4C)cc3)C2=O)n1] | 1-(6-(4-isopropyl-4H-1,2,4-triazol-3- yl)pyridin-2-yl)-3-(4-((3R,5S)-3,5- dimethylmorpholino)phenyl)imidazolidin-2- one |
| 1086. | 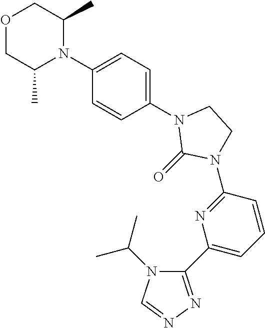 [SMILES: CC(C)n1cnnc1-c1cccc(N2CCN(c3ccc(N4[C@H](C)COC[C@H]4C)cc3)C2=O)n1] | 1-(6-(4-isopropyl-4H-1,2,4-triazol-3- yl)pyridin-2-yl)-3-(4-((3R,5R)-3,5- dimethylmorpholino)phenyl)imidazolidin-2- one |
| 1087. | 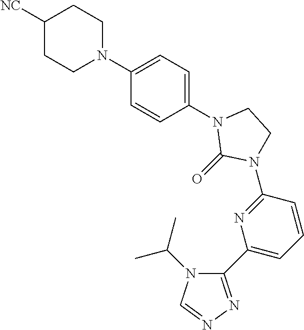 [SMILES: CC(C)n1cnnc1-c1cccc(N2CCN(c3ccc(N4CCC(C#N)CC4)cc3)C2=O)n1] | 1-(4-(3-(6-(4-isopropyl-4H-1,2,4-triazol-3- yl)pyridin-2-yl)-2-oxoimidazolidin-1- yl)phenyl)piperidine-4-carbonitrile |
| 1088. | 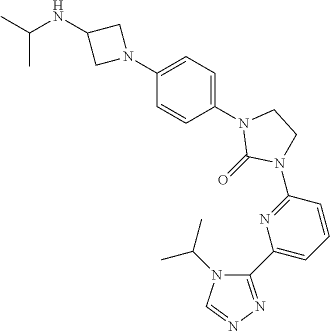 [SMILES: CC(C)NC1CN(c2ccc(N3CCN(c4cccc(-c5nncn5C(C)C)n4)C3=O)cc2)C1] | 1-(6-(4-isopropyl-4H-1,2,4-triazol-3- yl)pyridin-2-yl)-3-(4-(3- (isopropylamino)azetidin-1- yl)phenyl)imidazolidin-2-one |
| 1089. | 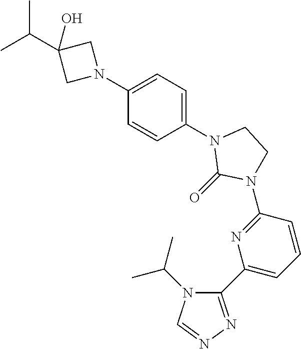 [SMILES: CC(C)n1cnnc1-c1cccc(N2CCN(c3ccc(N4CC(O)(C(C)C)C4)cc3)C2=O)n1] | 1-(4-(3-hydroxy-3-isopropylazetidin-1- yl)phenyl)-3-(6-(4-isopropyl-4H-1,2,4-triazol- 3-yl)pyridin-2-yl)imidazolidin-2-one |
| 1090. | 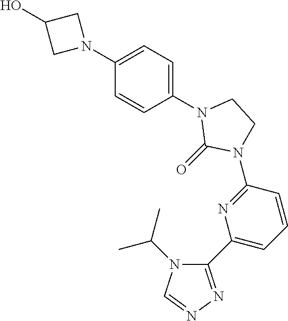 [SMILES: CC(C)n1cnnc1-c1cccc(N2CCN(c3ccc(N4CC(O)C4)cc3)C2=O)n1] | 1-(4-(3-hydroxyazetidin-1-yl)phenyl)-3-(6-(4- isopropyl-4H-1,2,4-triazol-3-yl)pyridin-2- yl)imidazolidin-2-one |
| 1091. | 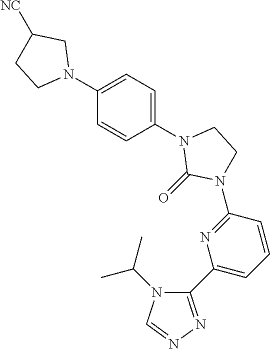 [SMILES: CC(C)n1cnnc1-c1cccc(N2CCN(c3ccc(N4CCC(C#N)C4)cc3)C2=O)n1] | 1-(4-(3-(6-(4-isopropyl-4H-1,2,4-triazol-3- yl)pyridin-2-yl)-2-oxoimidazolidin-1- yl)phenyl)pyrrolidine-3-carbonitrile |
| 1092. | 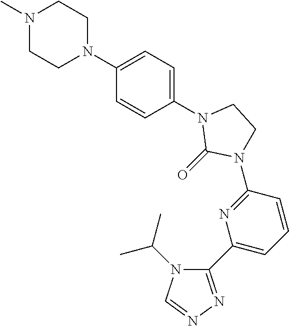 [SMILES: CC(C)n1cnnc1-c1cccc(N2CCN(c3ccc(N4CCN(C)CC4)cc3)C2=O)n1] | 1-(6-(4-isopropyl-4H-1,2,4-triazol-3- yl)pyridin-2-yl)-3-(4-(4-methylpiperazin-1- yl)phenyl)imidazolidin-2-one |
| 1093. | 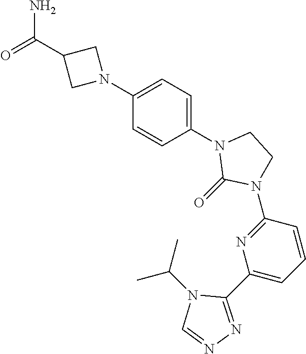 [SMILES: CC(C)n1cnnc1-c1cccc(N2CCN(c3ccc(N4CC(C(N)=O)C4)cc3)C2=O)n1] | 1-(4-(3-(6-(4-isopropyl-4H-1,2,4-triazol-3- yl)pyridin-2-yl)-2-oxoimidazolidin-1- yl)phenyl)azetidine-3-carboxamide |
| 1094. | 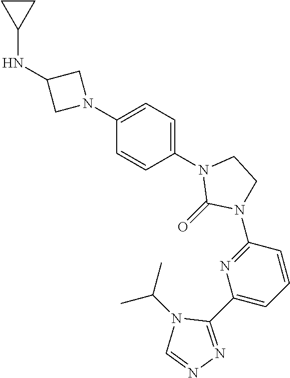 [SMILES: CC(C)n1cnnc1-c1cccc(N2CCN(c3ccc(N4CC(NC5CC5)C4)cc3)C2=O)n1] | 1-(4-(3-(cyclopropylamino)azetidin-1- yl)phenyl)-3-(6-(4-isopropyl-4H-1,2,4-triazol- 3-yl)pyridin-2-yl)imidazolidin-2-one |
| 1095. | 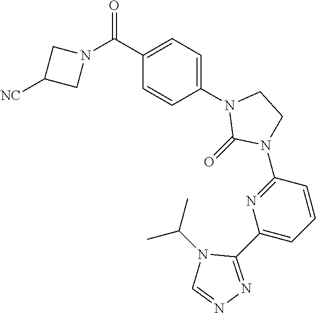 [SMILES: CC(C)n1cnnc1-c1cccc(N2CCN(c3ccc(C(=O)N4CC(C#N)C4)cc3)C2=O)n1] | 1-(4-(3-(6-(4-isopropyl-4H-1,2,4-triazol-3- yl)pyridin-2-yl)-2-oxoimidazolidin-1- yl)benzoyl)azetidine-3-carbonitrile |
| 1096. | 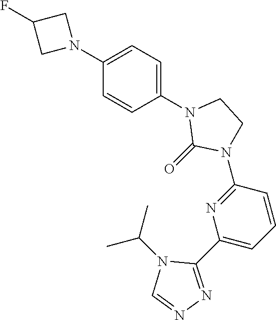 [SMILES: CC(C)n1cnnc1-c1cccc(N2CCN(c3ccc(N4CC(F)C4)cc3)C2=O)n1] | 1-(4-(3-fluoroazetidin-1-yl)phenyl)-3-(6-(4- isopropyl-4H-1,2,4-triazol-3-yl)pyridin-2- yl)imidazolidin-2-one |
| 1097. | 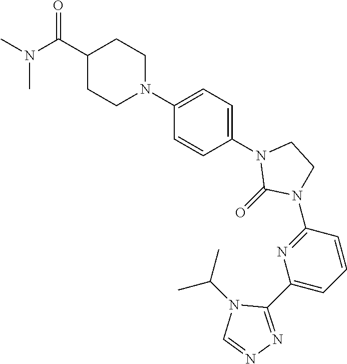 [SMILES: CC(C)n1cnnc1-c1cccc(N2CCN(c3ccc(N4CCC(C(=O)N(C)C)CC4)cc3)C2=O)n1] | 1-(4-(3-(6-(4-isopropyl-4H-1,2,4-triazol-3- yl)pyridin-2-yl)-2-oxoimidazolidin-1- yl)phenyl)-N,N-dimethylpiperidine-4- carboxamide |
| 1098. | 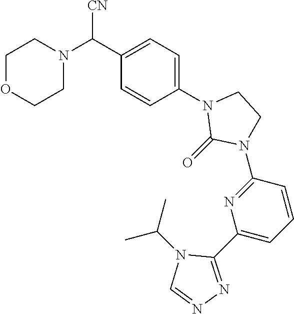 [SMILES: CC(C)n1cnnc1-c1cccc(N2CCN(c3ccc(C(C#N)N4CCOCC4)cc3)C2=O)n1] | 2-(4-(3-(6-(4-isopropyl-4H-1,2,4-triazol-3- yl)pyridin-2-yl)-2-oxoimidazolidin-1- yl)phenyl)-2-morpholinoacetonitrile |
| 1099. | 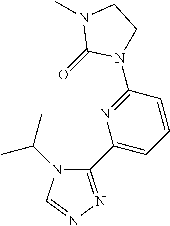 [SMILES: CC(C)n1cnnc1-c1cccc(N2CCN(C)C2=O)n1] | 1-(6-(4-isopropyl-4H-1,2,4-triazol-3- yl)pyridin-2-yl)-3-methylimidazolidin-2-one |
| 1100. | 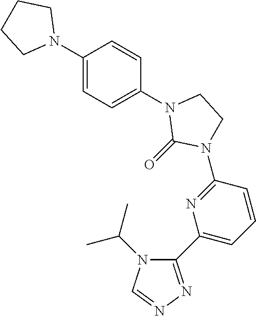 [SMILES: CC(C)n1cnnc1-c1cccc(N2CCN(c3ccc(N4CCCC4)cc3)C2=O)n1] | 1-(6-(4-isopropyl-4H-1,2,4-triazol-3- yl)pyridin-2-yl)-3-(4-(pyrrolidin-1- yl)phenyl)imidazolidin-2-one |
| 1101. | 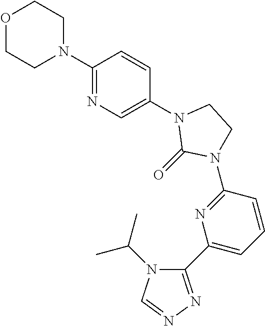 [SMILES: CC(C)n1cnnc1-c1cccc(N2CCN(c3ccc(N4CCOCC4)nc3)C2=O)n1] | 1-(6-(4-isopropyl-4H-1,2,4-triazol-3- yl)pyridin-2-yl)-3-(6-morpholinopyridin-3- yl)imidazolidin-2-one |
| 1102. | 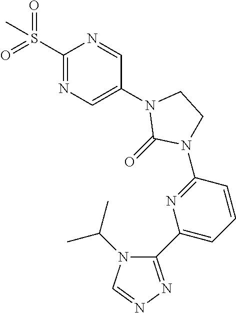 [SMILES: CC(C)n1cnnc1-c1cccc(N2CCN(c3cnc(S(C)(=O)=O)nc3)C2=O)n1] | 1-(6-(4-isopropyl-4H-1,2,4-triazol-3- yl)pyridin-2-yl)-3-(2- (methylsulfonyl)pyrimidin-5-yl)imidazolidin- 2-one |
| 1103. | 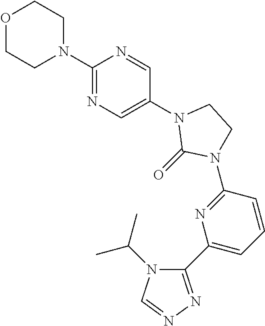 [SMILES: CC(C)n1cnnc1-c1cccc(N2CCN(c3cnc(N4CCOCC4)nc3)C2=O)n1] | 1-(6-(4-isopropyl-4H-1,2,4-triazol-3- yl)pyridin-2-yl)-3-(2-morpholinopyrimidin-5- yl)imidazolidin-2-one |
| 1104. | 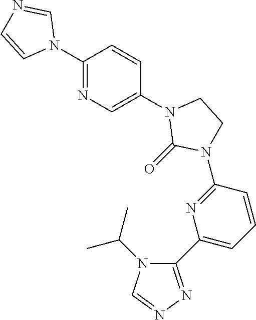 [SMILES: CC(C)n1cnnc1-c1cccc(N2CCN(c3ccc(-n4ccnc4)nc3)C2=O)n1] | 1-(6-(1H-imidazol-1-yl)pyridin-3-yl)-3-(6-(4- isopropyl-4H-1,2,4-triazol-3-yl)pyridin-2- yl)imidazolidin-2-one |
| 1105. | 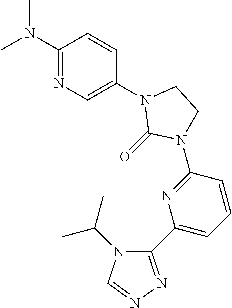 [SMILES: CC(C)n1cnnc1-c1cccc(N2CCN(c3ccc(N(C)C)nc3)C2=O)n1] | 1-(6-(dimethylamino)pyridin-3-yl)-3-(6-(4- isopropyl-4H-1,2,4-triazol-3-yl)pyridin-2- yl)imidazolidin-2-one |
| 1106. | 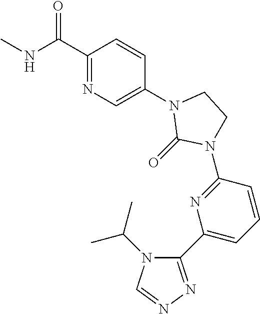 [SMILES: CNC(=O)c1ccc(N2CCN(c3cccc(-c4nncn4C(C)C)n3)C2=O)cn1] | 5-(3-(6-(4-isopropyl-4H-1,2,4-triazol-3- yl)pyridin-2-yl)-2-oxoimidazolidin-1-yl)-N- methylpyridine-2-carboxamide |
| 1107. | 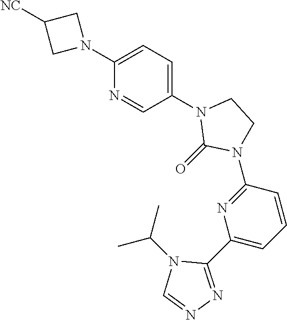 [SMILES: CC(C)n1cnnc1-c1cccc(N2CCN(c3ccc(N4CC(C#N)C4)nc3)C2=O)n1] | 1-(5-(3-(6-(4-isopropyl-4H-1,2,4-triazol-3- yl)pyridin-2-yl)-2-oxoimidazolidin-1- yl)pyridin-2-yl)azetidine-3-carbonitrile |
| 1108. | 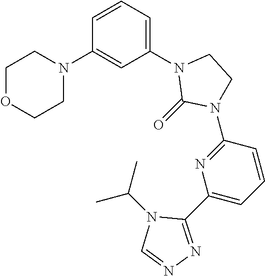 [SMILES: CC(C)n1cnnc1-c1cccc(N2CCN(c3cccc(N4CCOCC4)c3)C2=O)n1] | 1-(6-(4-isopropyl-4H-1,2,4-triazol-3- yl)pyridin-2-yl)-3-(3- morpholinophenyl)imidazolidin-2-one |
| 1109. | 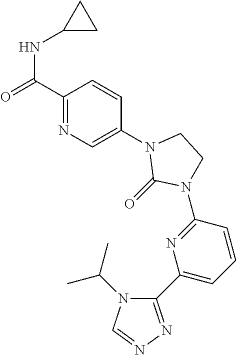 [SMILES: CC(C)n1cnnc1-c1cccc(N2CCN(c3ccc(C(=O)NC4CC4)nc3)C2=O)n1] | N-cyclopropyl-5-(3-(6-(4-isopropyl-4H-1,2,4- triazol-3-yl)pyridin-2-yl)-2-oxoimidazolidin-1- yl)pyridine-2-carboxamide |
| 1110. | 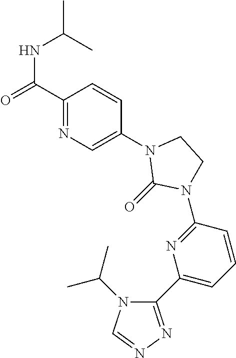 [SMILES: CC(C)NC(=O)c1ccc(N2CCN(c3cccc(-c4nncn4C(C)C)n3)C2=O)cn1] | N-isopropyl-5-(3-(6-(4-isopropyl-4H-1,2,4- triazol-3-yl)pyridin-2-yl)-2-oxoimidazolidin-1- yl)pyridine-2-carboxamide |
| 1111. | 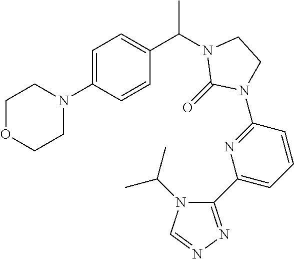 [SMILES: CC(c1ccc(N2CCOCC2)cc1)N1CCN(c2cccc(-c3nncn3C(C)C)n2)C1=O] | 1-(6-(4-isopropyl-4H-1,2,4-triazol-3- yl)pyridin-2-yl)-3-(1-(4- morpholinophenyl)ethyl)imidazolidin-2-one |
| 1112. | 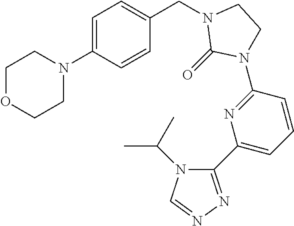 [SMILES: CC(C)n1cnnc1-c1cccc(N2CCN(Cc3ccc(N4CCOCC4)cc3)C2=O)n1] | 1-(4-morpholinobenzyl)-3-(6-(4-isopropyl-4H- 1,2,4-triazol-3-yl)pyridin-2-yl)imidazolidin-2- one |
| 1113. | 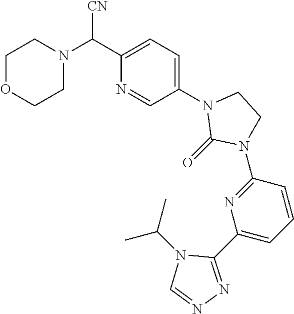 [SMILES: CC(C)n1cnnc1-c1cccc(N2CCN(c3ccc(C(C#N)N4CCOCC4)nc3)C2=O)n1] | 2-(5-(3-(6-(4-isopropyl-4H-1,2,4-triazol-3- yl)pyridin-2-yl)-2-oxoimidazolidin-1- yl)pyridin-2-yl)-2-morpholinoacetonitrile |
| 1114. | 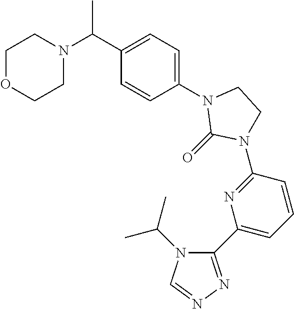 [SMILES: CC(c1ccc(N2CCN(c3cccc(-c4nncn4C(C)C)n3)C2=O)cc1)N1CCOCC1] | 1-(6-(4-isopropyl-4H-1,2,4-triazol-3- yl)pyridin-2-yl)-3-(4-(1- morpholinoethyl)phenyl)imidazolidin-2-one |
| 1115. | 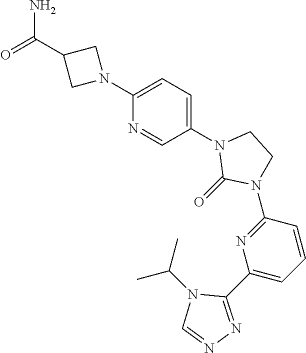 [SMILES: CC(C)n1cnnc1-c1cccc(N2CCN(c3ccc(N4CC(C(N)=O)C4)nc3)C2=O)n1] | 1-(5-(3-(6-(4-isopropyl-4H-1,2,4-triazol-3- yl)pyridin-2-yl)-2-oxoimidazolidin-1- yl)pyridin-2-yl)azetidine-3-carboxamide |
| 1116. | 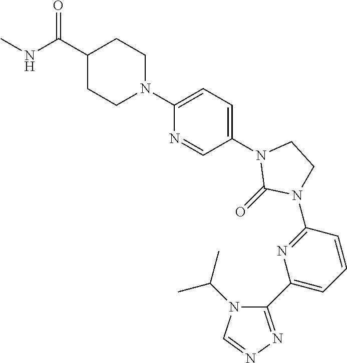 [SMILES: CNC(=O)C1CCN(c2ccc(N3CCN(c4cccc(-c5nncn5C(C)C)n4)C3=O)cn2)CC1] | 1-(5-(3-(6-(4-isopropyl-4H-1,2,4-triazol-3- yl)pyridin-2-yl)-2-oxoimidazolidin-1- yl)pyridin-2-yl)-N-methylpiperidine-4- carboxamide |
| 1117. | 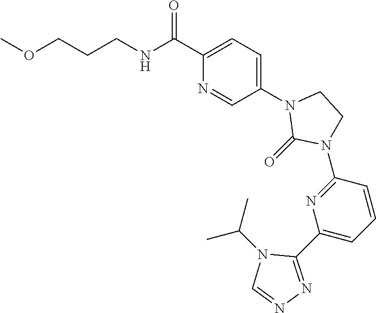 [SMILES: COCCCNC(=O)c1ccc(N2CCN(c3cccc(-c4nncn4C(C)C)n3)C2=O)cn1] | 5-(3-(6-(4-isopropyl-4H-1,2,4-triazol-3- yl)pyridin-2-yl)-2-oxoimidazolidin-1-yl)-N-(3- methoxypropyl)pyridine-2-carboxamide |
| 1118. | 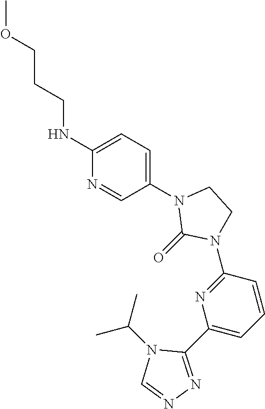 [SMILES: COCCCNc1ccc(N2CCN(c3cccc(-c4nncn4C(C)C)n3)C2=O)cn1] | 1-(6-(3-methoxypropylamino)pyridin-3-yl)-3- (6-(4-isopropyl-4H-1,2,4-triazol-3-yl)pyridin- 2-yl)imidazolidin-2-one |
| 1119. | 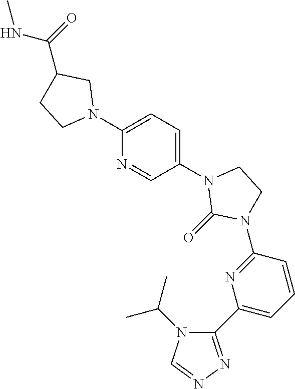 [SMILES: CNC(=O)C1CCN(c2ccc(N3CCN(c4cccc(-c5nncn5C(C)C)n4)C3=O)cn2)C1] | 1-(5-(3-(6-(4-isopropyl-4H-1,2,4-triazol-3- yl)pyridin-2-yl)-2-oxoimidazolidin-1- yl)pyridin-2-yl)-N-methylpyrrolidine-3- carboxamide |
| 1120. | 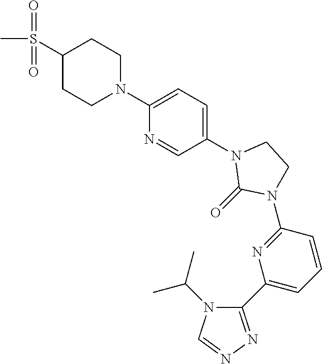 [SMILES: CC(C)n1cnnc1-c1cccc(N2CCN(c3ccc(N4CCC(S(C)(=O)=O)CC4)nc3)C2=O)n1] | 1-(6-(4-isopropyl-4H-1,2,4-triazol-3- yl)pyridin-2-yl)-3-(6-(4- (methylsulfonyl)piperidin-1-yl)pyridin-3- yl)imidazolidin-2-one |
| 1121. | 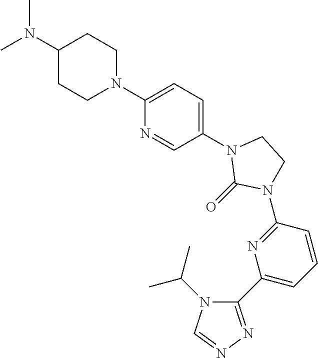 [SMILES: CC(C)n1cnnc1-c1cccc(N2CCN(c3ccc(N4CCC(N(C)C)CC4)nc3)C2=O)n1] | 1-(6-(4-(dimethylamino)piperidin-1-yl)pyridin- 3-yl)-3-(6-(4-isopropyl-4H-1,2,4-triazol-3- yl)pyridin-2-yl)imidazolidin-2-one |
| 1122. | 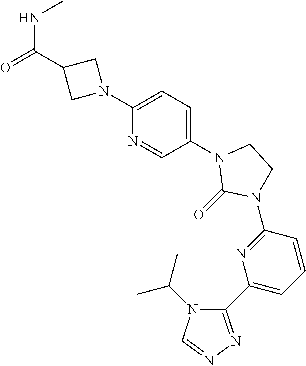 [SMILES: CNC(=O)C1CN(c2ccc(N3CCN(c4cccc(-c5nncn5C(C)C)n4)C3=O)cn2)C1] | 1-(5-(3-(6-(4-isopropyl-4H-1,2,4-triazol-3- yl)pyridin-2-yl)-2-oxoimidazolidin-1- yl)pyridin-2-yl)-N-methylazetidine-3- carboxamide |
| 1123. | 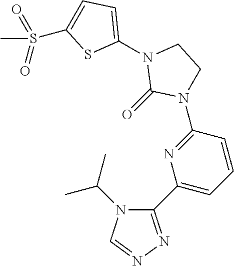 [SMILES: CC(C)n1cnnc1-c1cccc(N2CCN(c3ccc(S(C)(=O)=O)s3)C2=O)n1] | 1-(6-(4-isopropyl-4H-1,2,4-triazol-3- yl)pyridin-2-yl)-3-(5- (methylsulfonyl)thiophen-2-yl)imidazolidin-2- |
| 1124. | 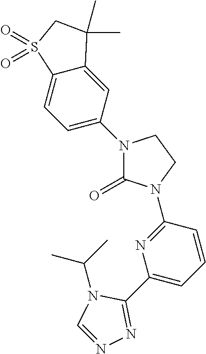 [SMILES: CC(C)n1cnnc1-c1cccc(N2CCN(c3ccc4c(c3)C(C)(C)CS4(=O)=O)C2=O)n1] | 1-(3,3-dimethyl-1,1-dioxido-2,3- dihydrobenzo[b]thiophen-5-yl)-3-(6-(4- isopropyl-4H-1,2,4-triazol-3-yl)pyridin-2- yl)imidazolidin-2-one |
| 1125. | 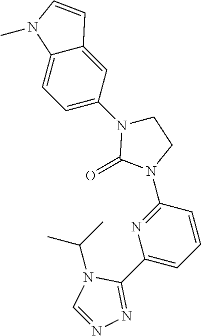 [SMILES: CC(C)n1cnnc1-c1cccc(N2CCN(c3ccc4c(ccn4C)c3)C2=O)n1] | 1-(6-(4-isopropyl-4H-1,2,4-triazol-3- yl)pyridin-2-yl)-3-(1-methyl-1H-indol-5- yl)imidazolidin-2-one |
| 1126. | 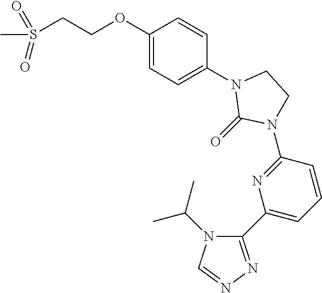 [SMILES: CC(C)n1cnnc1-c1cccc(N2CCN(c3ccc(OCCS(C)(=O)=O)cc3)C2=O)n1] | 1-(4-(2-(methylsulfonyl)ethoxy)phenyl)-3-(6- (4-isopropyl-4H-1,2,4-triazol-3-yl)pyridin-2- yl)imidazolidin-2-one |
| 1127. | 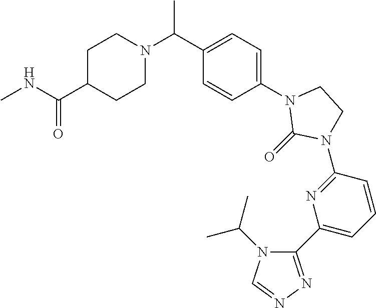 [SMILES: CNC(=O)C1CCN(C(C)c2ccc(N3CCN(c4cccc(-c5nncn5C(C)C)n4)C3=O)cc2)CC1] | 1-(1-(4-(3-(6-(4-isopropyl-4H-1,2,4-triazol-3- yl)pyridin-2-yl)-2-oxoimidazolidin-1- yl)phenyl)ethyl)-N-methylpiperidine-4- carboxamide |
| 1128. | 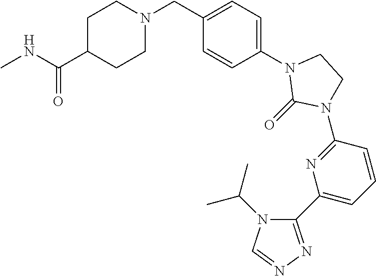 [SMILES: CNC(=O)C1CCN(Cc2ccc(N3CCN(c4cccc(-c5nncn5C(C)C)n4)C3=O)cc2)CC1] | 1-(4-(3-(6-(4-isopropyl-4H-1,2,4-triazol-3- yl)pyridin-2-yl)-2-oxoimidazolidin-1- yl)benzyl)-N-methylpiperidine-4-carboxamide |
| 1129. | 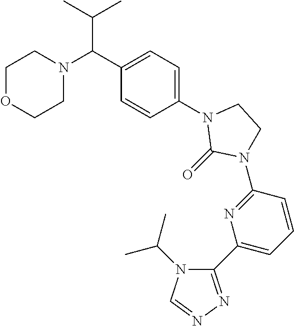 [SMILES: CC(C)C(c1ccc(N2CCN(c3cccc(-c4nncn4C(C)C)n3)C2=O)cc1)N1CCOCC1] | 1-(6-(4-isopropyl-4H-1,2,4-triazol-3- yl)pyridin-2-yl)-3-(4-(2-methyl-1- morpholinopropyl)phenyl)imidazolidin-2-one |
| 1130. | 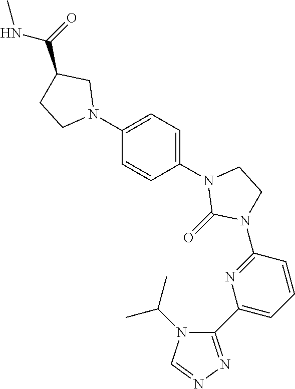 [SMILES: CNC(=O)[C@@H]1CCN(c2ccc(N3CCN(c4cccc(-c5nncn5C(C)C)n4)C3=O)cc2)C1] | (R)-1-(4-(3-(6-(4-isopropyl-4H-1,2,4-triazol-3- yl)pyridin-2-yl)-2-oxoimidazolidin-1- yl)phenyl)-N-methylpyrrolidine-3- carboxamide |
| 1131. | 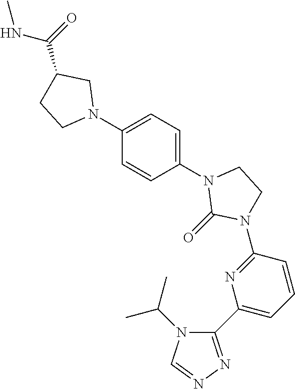 [SMILES: CNC(=O)[C@H]1CCN(c2ccc(N3CCN(c4cccc(-c5nncn5C(C)C)n4)C3=O)cc2)C1] | (S)-1-(4-(3-(6-(4-isopropyl-4H-1,2,4-triazol-3- yl)pyridin-2-yl)-2-oxoimidazolidin-1- yl)phenyl)-N-methylpyrrolidine-3- carboxamide |
| 1132. | 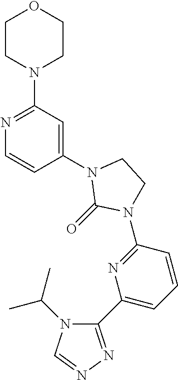 [SMILES: CC(C)n1cnnc1-c1cccc(N2CCN(c3ccnc(N4CCOCC4)c3)C2=O)n1] | 1-(6-(4-isopropyl-4H-1,2,4-triazol-3- yl)pyridin-2-yl)-3-(2-morpholinopyridin-4- yl)imidazolidin-2-one |
| 1133. | 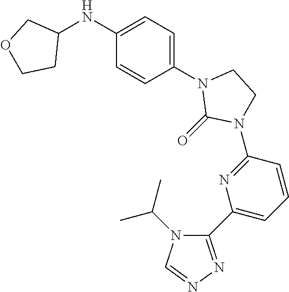 [SMILES: CC(C)n1cnnc1-c1cccc(N2CCN(c3ccc(NC4CCOC4)cc3)C2=O)n1] | 1-(6-(4-isopropyl-4H-1,2,4-triazol-3- yl)pyridin-2-yl)-3-(4-(tetrahydrofuran-3- ylamino)phenyl)imidazolidin-2-one |
| 1134. | 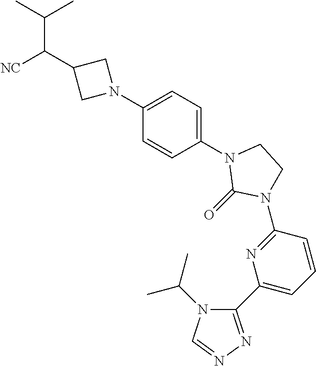 [SMILES: CC(C)C(C#N)C1CN(c2ccc(N3CCN(c4cccc(-c5nncn5C(C)C)n4)C3=O)cc2)C1] | 2-(1-(4-(3-(6-(4-isopropyl-4H-1,2,4-triazol-3- yl)pyridin-2-yl)-2-oxoimidazolidin-1- yl)phenyl)azetidin-3-yl)-3-methylbutanenitrile |
| 1135. | 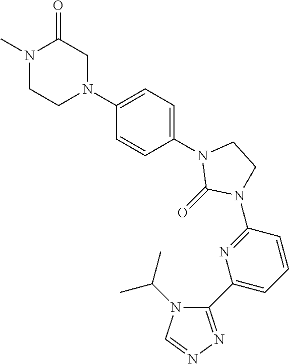 [SMILES: CC(C)n1cnnc1-c1cccc(N2CCN(c3ccc(N4CCN(C)C(=O)C4)cc3)C2=O)n1] | 4-(4-(3-(6-(4-isopropyl-4H-1,2,4-triazol-3- yl)pyridin-2-yl)-2-oxoimidazolidin-1- yl)phenyl)-1-methylpiperazin-2-one |
| 1136. | 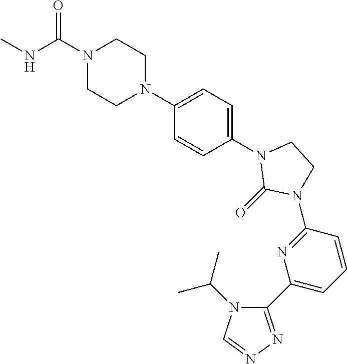 [SMILES: CNC(=O)N1CCN(c2ccc(N3CCN(c4cccc(-c5nncn5C(C)C)n4)C3=O)cc2)CC1] | 4-(4-(3-(6-(4-isopropyl-4H-1,2,4-triazol-3- yl)pyridin-2-yl)-2-oxoimidazolidin-1- yl)phenyl)-N-methylpiperazine-1-carboxamide |
| 1137. | 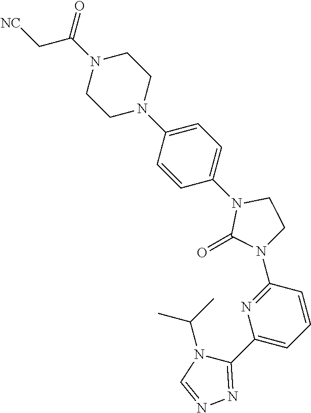 [SMILES: CC(C)n1cnnc1-c1cccc(N2CCN(c3ccc(N4CCN(C(=O)CC#N)CC4)cc3)C2=O)n1] | 3-(4-(4-(3-(6-(4-isopropyl-4H-1,2,4-triazol-3- yl)pyridin-2-yl)-2-oxoimidazolidin-1- yl)phenyl)piperazin-1-yl)-3-oxopropanenitrile |
| 1138. | 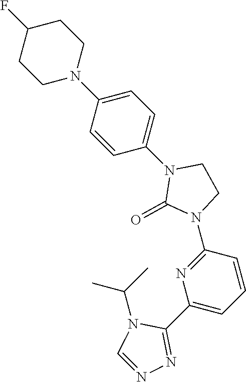 [SMILES: CC(C)n1cnnc1-c1cccc(N2CCN(c3ccc(N4CCC(F)CC4)cc3)C2=O)n1] | 1-(4-(4-fluoropiperidin-1-yl)phenyl)-3-(6-(4- isopropyl-4H-1,2,4-triazol-3-yl)pyridin-2- yl)imidazolidin-2-one |
| 1139. | 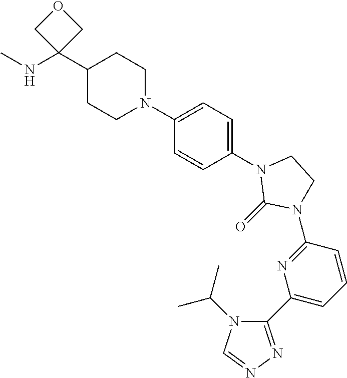 [SMILES: CNC1(C2CCN(c3ccc(N4CCN(c5cccc(-c6nncn6C(C)C)n5)C4=O)cc3)CC2)COC1] | 1-(6-(4-isopropyl-4H-1,2,4-triazol-3- yl)pyridin-2-yl)-3-(4-(4-(3- (methylamino)oxetan-3-yl)piperidin-1- yl)phenyl)imidazolidin-2-one |
| 1140. | 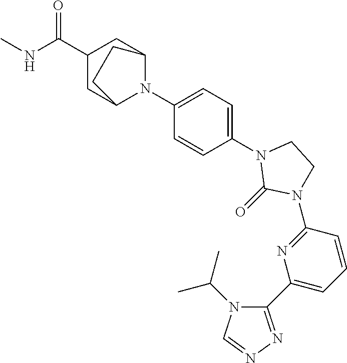 [SMILES: CNC(=O)C1CC2CCC(C1)N2c1ccc(N2CCN(c3cccc(-c4nncn4C(C)C)n3)C2=O)cc1] | 8-(4-(3-(6-(4-isopropyl-4H-1,2,4-triazol-3- yl)pyridin-2-yl)-2-oxoimidazolidin-1- yl)phenyl)-N-methyl-8-aza- bicyclo[3.2.1]octane-3-carboxamide |

The compounds of the present invention include:
1001. 1-(6-(1H-imidazol-1-yl)pyrimidin-4-yl)-3-(6-(4-isopropyl-4H-1,2,4-triazol-3-yl)pyridin-2-yl)imidazolidin-2-one;
1002. 1-(3-(1H-imidazol-1-yl)phenyl)-3-(6-(4-isopropyl-4H-1,2,4-triazol-3-yl)pyridin-2-yl)imidazolidin-2-one;
1003. 1-(4-(1H-imidazol-1-yl)pyridin-2-yl)-3-(6-(4-isopropyl-4H-1,2,4-triazol-3-yl)pyridin-2-yl)imidazolidin-2-one;
1004. 1-(6-(1H-imidazol-1-yl)pyrimidin-4-yl)-3-(6-(4-isopropyl-4H-1,2,4-triazol-3-yl)pyridin-2-yl)-1H-imidazol-2(3H)-one;
1005. 1-(3-(4-cyclopropyl-1H-imidazol-1-yl)phenyl)-3-(6-(4-isopropyl-4H-1,2,4-triazol-3-yl)pyridin-2-yl)imidazolidin-2-one;
1006. 1-(3-(1H-imidazol-1-yl)phenyl)-3-(6-(4-isopropyl-4H-1,2,4-triazol-3-yl)pyridin-2-yl)-1H-imidazol-2(3H)-one;
1007. 1-(6-(4-isopropyl-4H-1,2,4-triazol-3-yl)pyridin-2-yl)-3-(4′-(methylsulfonyl)-[1,1′-biphenyl]-3-yl)imidazolidin-2-one;
1008. 1-(2-fluoro-3-(1H-imidazol-1-yl)phenyl)-3-(6-(4-isopropyl-4H-1,2,4-triazol-3-yl)pyridin-2-yl)imidazolidin-2-one;
1009. 1-(6-(4-isopropyl-4H-1,2,4-triazol-3-yl)pyridin-2-yl)-3-(3-(methylsulfonyl)phenyl)imidazolidin-2-one;
1010. 1-(6-(4-isopropyl-4H-1,2,4-triazol-3-yl)pyridin-2-yl)-3-(3-(6-(methylsulfonyl)pyridin-3-yl)phenyl)imidazolidin-2-one;
1011. 1-(6-(4-isopropyl-4H-1,2,4-triazol-3-yl)pyridin-2-yl)-3-(3-(pyridin-4-yl)phenyl)imidazolidin-2-one;
1012. 1-(6-(4-isopropyl-4H-1,2,4-triazol-3-yl)pyridin-2-yl)-3-(3-(1-methyl-1H-pyrazol-4-yl)phenyl)imidazolidin-2-one;
1013. 1-(6-(4-isopropyl-4H-1,2,4-triazol-3-yl)pyridin-2-yl)-3-(4-(methylsulfonyl)phenyl)imidazolidin-2-one;
1014. 1-(6-(4-isopropyl-4H-1,2,4-triazol-3-yl)pyridin-2-yl)-3-(3-(1-(methylsulfonyl)-1H-pyrazol-4-yl)phenyl)imidazolidin-2-one;
1015. 3′-(3-(6-(4-isopropyl-4H-1,2,4-triazol-3-yl)pyridin-2-yl)-2-oxoimidazolidin-1-yl)-[1,1′-biphenyl]-4-carboxamide;
1016. 3′-(3-(6-(4-isopropyl-4H-1,2,4-triazol-3-yl)pyridin-2-yl)-2-oxoimidazolidin-1-yl)-[1,1′-biphenyl]-4-carbonitrile;
1017. 1-(6-(4-isopropyl-4H-1,2,4-triazol-3-yl)pyridin-2-yl)-3-(4′-(trifluoromethyl)-[1,1′-biphenyl]-3-yl)imidazolidin-2-one;
1018. 1-(6-(4-isopropyl-4H-1,2,4-triazol-3-yl)pyridin-2-yl)-3-(6-(methylsulfonyl)pyridin-3-yl)imidazolidin-2-one;
1019. 1-(6-(4-isopropyl-4H-1,2,4-triazol-3-yl)pyridin-2-yl)-3-(2-methoxy-5-(6-(methylsulfonyl)pyridin-3-yl)phenyl)imidazolidin-2-one;
1020. 4-(3-(6-(4-isopropyl-4H-1,2,4-triazol-3-yl)pyridin-2-yl)-2-oxoimidazolidin-1-yl)benzenesulfonamide;
1021. 1-(2-fluoro-5-(6-(methylsulfonyl)pyridin-3-yl)phenyl)-3-(6-(4-isopropyl-4H-1,2,4-triazol-3-yl)pyridin-2-yl)imidazolidin-2-one;
1022. 1-(6-(4-isopropyl-4H-1,2,4-triazol-3-yl)pyridin-2-yl)imidazolidin-2-one;
1023. 4-(3-(6-(4-isopropyl-4H-1,2,4-triazol-3-yl)pyridin-2-yl)-2-oxoimidazolidin-1-yl)benzenesulfonamide;
1024. 1-(6-(4-isopropyl-4H-1,2,4-triazol-3-yl)pyridin-2-yl)-3-phenylimidazolidin-2-one;
1025. 1-(4-(cyclopropylsulfonyl)phenyl)-3-(6-(4-isopropyl-4H-1,2,4-triazol-3-yl)pyridin-2-yl)imidazolidin-2-one;
1026. 1-(6-(4-isopropyl-4H-1,2,4-triazol-3-yl)pyridin-2-yl)-3-(4-methoxy-6-methyl-4′-(methylsulfonyl)-[1,1′-biphenyl]-3-yl)imidazolidin-2-one;
1027. 1-(6-(2-methoxyethylsulfonyl)pyridin-3-yl)-3-(6-(4-isopropyl-4H-1,2,4-triazol-3-yl)pyridin-2-yl)imidazolidin-2-one;
1028. 1-(6-(4-isopropyl-4H-1,2,4-triazol-3-yl)pyridin-2-yl)-3-(methylsulfonyl)imidazolidin-2-one;
1029. 1-(6-(4-isopropyl-4H-1,2,4-triazol-3-yl)pyridin-2-yl)-3-(3-methyl-4-(methylsulfonyl)phenyl)imidazolidin-2-one;
1030. 1-(4-hydroxyphenyl)-3-(6-(4-isopropyl-4H-1,2,4-triazol-3-yl)pyridin-2-yl)imidazolidin-2-one
1031. 1-(6-(4-isopropyl-4H-1,2,4-triazol-3-yl)pyridin-2-yl)-3-(2-methoxy-4-methyl-5-(methylsulfonyl)phenyl)imidazolidin-2-one;
1032. 1-(2-fluoro-4-(methylsulfonyl)phenyl)-3-(6-(4-isopropyl-4H-1,2,4-triazol-3-yl)pyridin-2-yl)imidazolidin-2-one;
1033. 1-(4-(trifluoromethyl)phenyl)-3-(6-(4-isopropyl-4H-1,2,4-triazol-3-yl)pyridin-2-yl)imidazolidin-2-one;
1034. 1-(6-(4-isopropyl-4H-1,2,4-triazol-3-yl)pyridin-2-yl)-3-(pyridin-4-yl)imidazolidin-2-one;
1035. 4-(3-(6-(4-isopropyl-4H-1,2,4-triazol-3-yl)pyridin-2-yl)-2-oxoimidazolidin-1-yl)benzonitrile;
1036. 1-(4-(1H-imidazol-1-yl)phenyl)-3-(6-(4-isopropyl-4H-1,2,4-triazol-3-yl)pyridin-2-yl)imidazolidin-2-one;
1037. 1-(4-(cyclopropanecarbonyl)phenyl)-3-(6-(4-isopropyl-4H-1,2,4-triazol-3-yl)pyridin-2-yl)imidazolidin-2-one;
1038. 1-(3-fluoro-4-(methylsulfonyl)phenyl)-3-(6-(4-isopropyl-4H-1,2,4-triazol-3-yl)pyridin-2-yl)imidazolidin-2-one;
1039. 1-(6-(4-isopropyl-4H-1,2,4-triazol-3-yl)pyridin-2-yl)-3-(4-methoxyphenyl)imidazolidin-2-one;
1040. 1-(4-(4-cyclopropyl-1H-imidazol-1-yl)phenyl)-3-(6-(4-isopropyl-4H-1,2,4-triazol-3-yl)pyridin-2-yl)imidazolidin-2-one;
1041. 1-(4-(dimethylamino)phenyl)-3-(6-(4-isopropyl-4H-1,2,4-triazol-3-yl)pyridin-2-yl)imidazolidin-2-one;
1042. 1-(6-(4-isopropyl-4H-1,2,4-triazol-3-yl)pyridin-2-yl)-3-(2-methyl-4-(methylsulfonyl)phenyl)imidazolidin-2-one;
1043. 1-(6-(4-isopropyl-4H-1,2,4-triazol-3-yl)pyridin-2-yl)-3-(4-morpholinophenyl)imidazolidin-2-one;
1044. N-(4-(3-(6-(4-isopropyl-4H-1,2,4-triazol-3-yl)pyridin-2-yl)-2-oxoimidazolidin-1-yl)phenyl)methanesulfonamide;
1045. 4-(3-(6-(4-isopropyl-4H-1,2,4-triazol-3-yl)pyridin-2-yl)-2-oxoimidazolidin-1-yl)-N-methylbenzenesulfonamide;
1046. N-cyclopropyl-6-(3-(6-(4-isopropyl-4H-1,2,4-triazol-3-yl)pyridin-2-yl)-2-oxoimidazolidin-1-yl)benzo[d]thiazol-2-aminium 2,2,2-trifluoroacetate;
1047. 1-(6-(4-isopropyl-4H-1,2,4-triazol-3-yl)pyridin-2-yl)-3-(4-((methylsulfonyl)methyl)phenyl)imidazolidin-2-one;
1048. N-cyclopropyl-4-(3-(6-(4-isopropyl-4H-1,2,4-triazol-3-yl)pyridin-2-yl)-2-oxoimidazolidin-1-yl)benzamide;
1049. 1-(6-(4-cyclopropyl-4H-1,2,4-triazol-3-yl)pyridin-2-yl)-3-(4-(methylsulfonyl)phenyl)imidazolidin-2-one;
1050. 1-(4-(2-methoxyethylsulfonyl)phenyl)-3-(6-(4-isopropyl-4H-1,2,4-triazol-3-yl)pyridin-2-yl)imidazolidin-2-one;
1051. 1-(4-(cyclopropyl(hydroxy)methyl)phenyl)-3-(6-(4-isopropyl-4H-1,2,4-triazol-3-yl)pyridin-2-yl)imidazolidin-2-one;
1052. 2-(4-(3-(6-(4-isopropyl-4H-1,2,4-triazol-3-yl)pyridin-2-yl)-2-oxoimidazolidin-1-yl)phenylamino)-2-cyclopropylacetamide;
1053. 3-(3-(6-(3-(4-(methylsulfonyl)phenyl)-2-oxoimidazolidin-1-yl)pyridin-2-yl)-4H-1,2,4-triazol-4-yl)butanenitrile;
1054. 1-(6-(4-isopropyl-4H-1,2,4-triazol-3-yl)pyridin-2-yl)-3-(3-methoxy-4-(methylsulfonyl)phenyl)imidazolidin-2-one;
1055. 1-(6-(4-isopropyl-4H-1,2,4-triazol-3-yl)pyridin-2-yl)-3-(2-methoxy-5-methyl-4-(methylsulfonyl)phenyl)imidazolidin-2-one;
1056. 2-(4-(3-(6-(4-isopropyl-4H-1,2,4-triazol-3-yl)pyridin-2-yl)-2-oxoimidazolidin-1-yl)phenylamino)-2-cyclopropylacetonitrile;
1057. 1-(6-(4-(1-hydroxypropan-2-yl)-4H-1,2,4-triazol-3-yl)pyridin-2-yl)-3-(4-(methylsulfonyl)phenyl)imidazolidin-2-one;
1058. 1-(6-(4-isopropyl-4H-1,2,4-triazol-3-yl)pyridin-2-yl)-3-(4-(methylsulfonyl)-3-(pyrrolidin-1-yl)phenyl)imidazolidin-2-one;
1059. 1-(3-cyclopropyl-4-(methylsulfonyl)phenyl)-3-(6-(4-isopropyl-4H-1,2,4-triazol-3-yl)pyridin-2-yl)imidazolidin-2-one;
1060. 1-(3-cyclopropyl-4-fluorophenyl)-3-(6-(4-isopropyl-4H-1,2,4-triazol-3-yl)pyridin-2-yl)imidazolidin-2-one;
1061. 1-(6-(4-(1,1,1-trifluoropropan-2-yl)-4H-1,2,4-triazol-3-yl)pyridin-2-yl)-3-(4-(methylsulfonyl)phenyl)imidazolidin-2-one;
1062. 1-(3-isopropyl-4-(methylsulfonyl)phenyl)-3-(6-(4-isopropyl-4H-1,2,4-triazol-3-yl)pyridin-2-yl)imidazolidin-2-one;
1063. 1-(6-(4-isopropyl-4H-1,2,4-triazol-3-yl)pyridin-2-yl)-3-(4-(methylsulfonyl)-3-(piperidin-1-yl)phenyl)imidazolidin-2-one;
1064. 1-(5-(4-isopropyl-4H-1,2,4-triazol-3-yl)pyridin-2-yl)-3-(4-(methylsulfonyl)phenyl)imidazolidin-2-one;
1065. 1-(6-(4-isopropyl-4H-1,2,4-triazol-3-yl)pyridin-2-yl)-3-(4-(methylsulfonyl)phenyl)imidazolidin-2-one;
1066. 1-(4-(tetrahydro-2H-pyran-4-yl)phenyl)-3-(6-(4-isopropyl-4H-1,2,4-triazol-3-yl)pyridin-2-yl)imidazolidin-2-one;
1067. 1-(4-(4-hydroxypiperidin-1-yl)phenyl)-3-(6-(4-isopropyl-4H-1,2,4-triazol-3-yl)pyridin-2-yl)imidazolidin-2-one;
1068. 4-(4-(3-(6-(4-isopropyl-4H-1,2,4-triazol-3-yl)pyridin-2-yl)-2-oxoimidazolidin-1-yl)phenyl)-5-methylmorpholin-3-one;
1069. 1-(6-(4-isopropyl-4H-1,2,4-triazol-3-yl)pyridin-2-yl)-3-(4-(3-methylmorpholino)phenyl)imidazolidin-2-one;
1070. 1-(6-(4-isopropyl-4H-1,2,4-triazol-3-yl)pyridin-2-yl)-3-(4-(4,4-dimethylpiperidin-1-yl)phenyl)imidazolidin-2-one;
1071. 1-(4-(3-(6-(4-isopropyl-4H-1,2,4-triazol-3-yl)pyridin-2-yl)-2-oxoimidazolidin-1-yl)phenyl)azetidine-3-carbonitrile;
1072. 1-(6-(4-hydroxypiperidin-1-yl)pyridin-3-yl)-3-(6-(4-isopropyl-4H-1,2,4-triazol-3-yl)pyridin-2-yl)imidazolidin-2-one;
1073. 1-(6-(4-isopropyl-4H-1,2,4-triazol-3-yl)pyridin-2-yl)-3-(4-(morpholinosulfonyl)phenyl)imidazolidin-2-one;
1074. 1-(4-(2-oxa-6-azaspiro[3.3]heptan-6-yl)phenyl)-3-(6-(4-isopropyl-4H-1,2,4-triazol-3-yl)pyridin-2-yl)imidazolidin-2-one;
1075. 1-(3-fluoro-4-morpholinophenyl)-3-(6-(4-isopropyl-4H-1,2,4-triazol-3-yl)pyridin-2-yl)imidazolidin-2-one;
1076. 4-(4-(3-(6-(4-isopropyl-4H-1,2,4-triazol-3-yl)pyridin-2-yl)-2-oxoimidazolidin-1-yl)phenyl)-6-methylmorpholin-3-one;
1077. 1-(6-(4-isopropyl-4H-1,2,4-triazol-3-yl)pyridin-2-yl)-3-(4-(morpholine-4-carbonyl)phenyl)imidazolidin-2-one;
1078. 1-(6-(4-isopropyl-4H-1,2,4-triazol-3-yl)pyridin-2-yl)-3-(4-(2-methylmorpholino)phenyl)imidazolidin-2-one;
1079. 1-(6-(4-isopropyl-4H-1,2,4-triazol-3-yl)pyridin-2-yl)-3-(3-methyl-4-morpholinophenyl)imidazolidin-2-one;
1080. 1-(6-(4-isopropyl-4H-1,2,4-triazol-3-yl)pyridin-2-yl)-3-(4-(morpholinomethyl)phenyl)imidazolidin-2-one;
1081. 1-(6-(4-isopropyl-4H-1,2,4-triazol-3-yl)pyridin-2-yl)-3-(4-(tetrahydro-2H-pyran-4-ylamino)phenyl)imidazolidin-2-one;
1082. 1-(4-(3-(6-(4-isopropyl-4H-1,2,4-triazol-3-yl)pyridin-2-yl)-2-oxoimidazolidin-1-yl)phenyl)-N-methylpiperidine-4-carboxamide;
1083. 1-(4-(2,2,2-trifluoro-1-hydroxyethyl)phenyl)-3-(6-(4-isopropyl-4H-1,2,4-triazol-3-yl)pyridin-2-yl)imidazolidin-2-one;
1084. 1-(6-(4-isopropyl-4H-1,2,4-triazol-3-yl)pyridin-2-yl)-3-(4-(1-(methylsulfonyl)piperidin-4-yl)phenyl)imidazolidin-2-one;
1085. 1-(6-(4-isopropyl-4H-1,2,4-triazol-3-yl)pyridin-2-yl)-3-(4-((3R,5S)-3,5-dimethylmorpholino)phenyl)imidazolidin-2-one;
1086. 1-(6-(4-isopropyl-4H-1,2,4-triazol-3-yl)pyridin-2-yl)-3-(4-((3R,5R)-3,5-dimethylmorpholino)phenyl)imidazolidin-2-one;
1087. 1-(4-(3-(6-(4-isopropyl-4H-1,2,4-triazol-3-yl)pyridin-2-yl)-2-oxoimidazolidin-1-yl)phenyl)piperidine-4-carbonitrile;
1088. 1-(6-(4-isopropyl-4H-1,2,4-triazol-3-yl)pyridin-2-yl)-3-(4-(3-(isopropylamino)azetidin-1-yl)phenyl)imidazolidin-2-one;
1089. 1-(4-(3-hydroxy-3-isopropylazetidin-1-yl)phenyl)-3-(6-(4-isopropyl-4H-1,2,4-triazol-3-yl)pyridin-2-yl)imidazolidin-2-one;
1090. 1-(4-(3-hydroxyazetidin-1-yl)phenyl)-3-(6-(4-isopropyl-4H-1,2,4-triazol-3-yl)pyridin-2-yl)imidazolidin-2-one;
1091. 1-(4-(3-(6-(4-isopropyl-4H-1,2,4-triazol-3-yl)pyridin-2-yl)-2-oxoimidazolidin-1-yl)phenyl)pyrrolidine-3-carbonitrile;
1092. 1-(6-(4-isopropyl-4H-1,2,4-triazol-3-yl)pyridin-2-yl)-3-(4-(4-methylpiperazin-1-yl)phenyl)imidazolidin-2-one;
1093. 1-(4-(3-(6-(4-isopropyl-4H-1,2,4-triazol-3-yl)pyridin-2-yl)-2-oxoimidazolidin-1-yl)phenyl)azetidine-3-carboxamide;
1094. 1-(4-(3-(cyclopropylamino)azetidin-1-yl)phenyl)-3-(6-(4-isopropyl-4H-1,2,4-triazol-3-yl)pyridin-2-yl)imidazolidin-2-one;
1095. 1-(4-(3-(6-(4-isopropyl-4H-1,2,4-triazol-3-yl)pyridin-2-yl)-2-oxoimidazolidin-1-yl)benzoyl)azetidine-3-carbonitrile;
1096. 1-(4-(3-fluoroazetidin-1-yl)phenyl)-3-(6-(4-isopropyl-4H-1,2,4-triazol-3-yl)pyridin-2-yl)imidazolidin-2-one;
1097. 1-(4-(3-(6-(4-isopropyl-4H-1,2,4-triazol-3-yl)pyridin-2-yl)-2-oxoimidazolidin-1-yl)phenyl)-N,N-dimethylpiperidine-4-carboxamide;
1098. 2-(4-(3-(6-(4-isopropyl-4H-1,2,4-triazol-3-yl)pyridin-2-yl)-2-oxoimidazolidin-1-yl)phenyl)-2-morpholinoacetonitrile;
1099. 1-(6-(4-isopropyl-4H-1,2,4-triazol-3-yl)pyridin-2-yl)-3-methylimidazolidin-2-one;
1100. 1-(6-(4-isopropyl-4H-1,2,4-triazol-3-yl)pyridin-2-yl)-3-(4-(pyrrolidin-1-yl)phenyl)imidazolidin-2-one;
1101. 1-(6-(4-isopropyl-4H-1,2,4-triazol-3-yl)pyridin-2-yl)-3-(6-morpholinopyridin-3-yl)imidazolidin-2-one;
1102. 1-(6-(4-isopropyl-4H-1,2,4-triazol-3-yl)pyridin-2-yl)-3-(2-(methylsulfonyl)pyrimidin-5-yl)imidazolidin-2-one;
1103. 1-(6-(4-isopropyl-4H-1,2,4-triazol-3-yl)pyridin-2-yl)-3-(2-morpholinopyrimidin-5-yl)imidazolidin-2-one;
1104. 1-(6-(1H-imidazol-1-yl)pyridin-3-yl)-3-(6-(4-isopropyl-4H-1,2,4-triazol-3-yl)pyridin-2-yl)imidazolidin-2-one;
1105. 1-(6-(dimethylamino)pyridin-3-yl)-3-(6-(4-isopropyl-4H-1,2,4-triazol-3-yl)pyridin-2-yl)imidazolidin-2-one;
1106. 5-(3-(6-(4-isopropyl-4H-1,2,4-triazol-3-yl)pyridin-2-yl)-2-oxoimidazolidin-1-yl)-N-methylpyridine-2-carboxamide;
1107. 1-(5-(3-(6-(4-isopropyl-4H-1,2,4-triazol-3-yl)pyridin-2-yl)-2-oxoimidazolidin-1-yl)pyridin-2-yl)azetidine-3-carbonitrile;
1108. 1-(6-(4-isopropyl-4H-1,2,4-triazol-3-yl)pyridin-2-yl)-3-(3-morpholinophenyl)imidazolidin-2-one;
1109. N-cyclopropyl-5-(3-(6-(4-isopropyl-4H-1,2,4-triazol-3-yl)pyridin-2-yl)-2-oxoimidazolidin-1-yl)pyridine-2-carboxamide;
1110. N-isopropyl-5-(3-(6-(4-isopropyl-4H-1,2,4-triazol-3-yl)pyridin-2-yl)-2-oxoimidazolidin-1-yl)pyridine-2-carboxamide;
1111. 1-(6-(4-isopropyl-4H-1,2,4-triazol-3-yl)pyridin-2-yl)-3-(1-(4-morpholinophenyl)ethyl)imidazolidin-2-one;
1112. 1-(4-morpholinobenzyl)-3-(6-(4-isopropyl-4H-1,2,4-triazol-3-yl)pyridin-2-yl)imidazolidin-2-one;
1113. 2-(5-(3-(6-(4-isopropyl-4H-1,2,4-triazol-3-yl)pyridin-2-yl)-2-oxoimidazolidin-1-yl)pyridin-2-yl)-2-morpholinoacetonitrile;
1114. 1-(6-(4-isopropyl-4H-1,2,4-triazol-3-yl)pyridin-2-yl)-3-(4-(1-morpholinoethyl)phenyl)imidazolidin-2-one;
1115. 1-(5-(3-(6-(4-isopropyl-4H-1,2,4-triazol-3-yl)pyridin-2-yl)-2-oxoimidazolidin-1-yl)pyridin-2-yl)azetidine-3-carboxamide;
1116. 1-(5-(3-(6-(4-isopropyl-4H-1,2,4-triazol-3-yl)pyridin-2-yl)-2-oxoimidazolidin-1-yl)pyridin-2-yl)-N-methylpiperidine-4-carboxamide;
1117. 5-(3-(6-(4-isopropyl-4H-1,2,4-triazol-3-yl)pyridin-2-yl)-2-oxoimidazolidin-1-yl)-N-(3-methoxypropyl)pyridine-2-carboxamide;
1118. 1-(6-(3-methoxypropylamino)pyridin-3-yl)-3-(6-(4-isopropyl-4H-1,2,4-triazol-3-yl)pyridin-2-yl)imidazolidin-2-one;
1119. 1-(5-(3-(6-(4-isopropyl-4H-1,2,4-triazol-3-yl)pyridin-2-yl)-2-oxoimidazolidin-1-yl)pyridin-2-yl)-N-methylpyrrolidine-3-carboxamide;
1120. 1-(6-(4-isopropyl-4H-1,2,4-triazol-3-yl)pyridin-2-yl)-3-(6-(4-(methylsulfonyl)piperidin-1-yl)pyridin-3-yl)imidazolidin-2-one;
1121. 1-(6-(4-(dimethylamino)piperidin-1-yl)pyridin-3-yl)-3-(6-(4-isopropyl-4H-1,2,4-triazol-3-yl)pyridin-2-yl)imidazolidin-2-one;
1122. 1-(5-(3-(6-(4-isopropyl-4H-1,2,4-triazol-3-yl)pyridin-2-yl)-2-oxoimidazolidin-1-yl)pyridin-2-yl)-N-methylazetidine-3-carboxamide;
1123. 1-(6-(4-isopropyl-4H-1,2,4-triazol-3-yl)pyridin-2-yl)-3-(5-(methylsulfonyl)thiophen-2-yl)imidazolidin-2-one;
1124. 1-(3,3-dimethyl-1,1-dioxido-2,3-dihydrobenzo[b]thiophen-5-yl)-3-(6-(4-isopropyl-4H-1,2,4-triazol-3-yl)pyridin-2-yl)imidazolidin-2-one;
1125. 1-(6-(4-isopropyl-4H-1,2,4-triazol-3-yl)pyridin-2-yl)-3-(1-methyl-1H-indo1-5-yl)imidazolidin-2-one;
1126. 1-(4-(2-(methylsulfonyl)ethoxy)phenyl)-3-(6-(4-isopropyl-4H-1,2,4-triazol-3-yl)pyridin-2-yl)imidazolidin-2-one;
1127. 1-(1-(4-(3-(6-(4-isopropyl-4H-1,2,4-triazol-3-yl)pyridin-2-yl)-2-oxoimidazolidin-1-yl)phenyl)ethyl)-N-methylpiperidine-4-carboxamide;
1128. 1-(4-(3-(6-(4-isopropyl-4H-1,2,4-triazol-3-yl)pyridin-2-yl)-2-oxoimidazolidin-1-yl)benzyl)-N-methylpiperidine-4-carboxamide;
1129. 1-(6-(4-isopropyl-4H-1,2,4-triazol-3-yl)pyridin-2-yl)-3-(4-(2-methyl-1-morpholinopropyl)phenyl)imidazolidin-2-one;
1130. (R)-1-(4-(3-(6-(4-isopropyl-4H-1,2,4-triazol-3-yl)pyridin-2-yl)-2-oxoimidazolidin-1-yl)phenyl)-N-methylpyrrolidine-3-carboxamide;
1131. (S)-1-(4-(3-(6-(4-isopropyl-4H-1,2,4-triazol-3-yl)pyridin-2-yl)-2-oxoimidazolidin-1-yl)phenyl)-N-methylpyrrolidine-3-carboxamide;
1132. 1-(6-(4-isopropyl-4H-1,2,4-triazol-3-yl)pyridin-2-yl)-3-(2-morpholinopyridin-4-yl)imidazolidin-2-one;
1133. 1-(6-(4-isopropyl-4H-1,2,4-triazol-3-yl)pyridin-2-yl)-3-(4-(tetrahydrofuran-3-ylamino)phenyl)imidazolidin-2-one;
1134. 2-(1-(4-(3-(6-(4-isopropyl-4H-1,2,4-triazol-3-yl)pyridin-2-yl)-2-oxoimidazolidin-1-yl)phenyl)azetidin-3-yl)-3-methylbutanenitrile;
1135. 4-(4-(3-(6-(4-isopropyl-4H-1,2,4-triazol-3-yl)pyridin-2-yl)-2-oxoimidazolidin-1-yl)phenyl)-1-methylpiperazin-2-one;
1136. 4-(4-(3-(6-(4-isopropyl-4H-1,2,4-triazol-3-yl)pyridin-2-yl)-2-oxoimidazolidin-1-yl)phenyl)-N-methylpiperazine-1-carboxamide;
1137. 3-(4-(4-(3-(6-(4-isopropyl-4H-1,2,4-triazol-3-yl)pyridin-2-yl)-2-oxoimidazolidin-1-yl)phenyl)piperazin-1-yl)-3-oxopropanenitrile;
1138. 1-(4-(4-fluoropiperidin-1-yl)phenyl)-3-(6-(4-isopropyl-4H-1,2,4-triazol-3-yl)pyridin-2-yl)imidazolidin-2-one;
1139. 1-(6-(4-isopropyl-4H-1,2,4-triazol-3-yl)pyridin-2-yl)-3-(4-(4-(3-(methylamino)oxetan-3-yl)piperidin-1-yl)phenyl)imidazolidin-2-one;
1140. 8-(4-(3-(6-(4-isopropyl-4H-1,2,4-triazol-3-yl)pyridin-2-yl)-2-oxoimidazolidin-1-yl)phenyl)-N-methyl-8-aza-bicyclo[3.2.1]octane-3-carboxamide.

The present invention includes compound of Formula II, their stereoisomers, salts and solvates thereof;

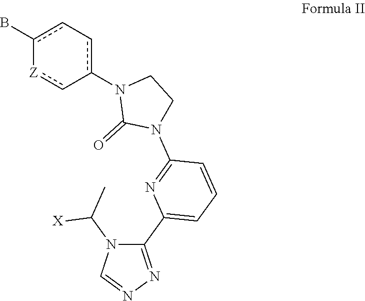

Wherein;
indicates an optional bond;

B is absent, H, SO₂, SO₂(CH₂)n; SO₂R₁, SO₂(CH₂)nOR₁, SO₂NHR₁, NH₂(CH₂)n, CO NH₂(CH₂)n, NH₂(CH₂)n, NH₂(CH₂)nR₁CONH₂, NH₂(CH₂)nCN, NHCHR₁CN, NHR₁, SO₂Me, 3 to 8 carbocyclic ring optionally substituted by R1 or R2 or R3, 3 to 8 membered heterocyclic ring containing 1 to 3 heteroatoms selected from the group comprising O, N, S which may be optionally substituted by R1 or R2 or R3;

X is H, CH₃, (CH₂)n, C₁-C₆ straight or branched or cyclic alkyl, CN, (CH₂)CN, OH, (CH₂)nOH, CF₃;

R1, R2 and R3 are independently selected from the group comprising H, halo, CN, CF₃, CH₃, hydroxyl, C₁-C₆ alkyl, C₁ to C₆ alkoxy , C1-C6 alkoxyalkyl and C1-C6 cyanoalkyl, amino, SO₂, SO₂C₁-C₆ Alkyl, CH₂CF₃, —(CH₂)nCN, —CH₂OMe, —CH₂SO₂Me, NR₄R₅, CONR₄R₅, C₁-C₆ straight or branched or cyclic alkyl, C₂-C₆ straight or branched alkenyl, C₂-C₆ straight or branched alkynyl, halo-C₁-C₆ alkyl, C₁-C₆ alkyloxy; C₁-C₆ alkylamino, heteroalkyl, C₃-C₈ cycloalkyl, C₃-C₈cycloalkenyl, C₃-C₈heterocycloalkyl, C₃-C₈heterocycloalkenyl, carbocycyl, aryl, and heteroaryl; oxidised heteroaryl, oxidise heterocyclyl, heteroaryl or heterocyclyl that may be optionally substituted with R4 and R5;

R4 and R5 may be selected from H, C₁-C₆ alkyl and C₃-C₈ cycloalkyl, NH₂, NHC₁-C₆ alkyl

Z can be CH or N;

n is 0 to 3.

The compounds of formula II, may be selected from the group comprising:

1043: 1-(6-(4-isopropyl-4H-1,2,4-triazol-3-yl)pyridin-2-yl)-3-(4-morpholinophenyl)imidazolidin-2-one;

1071: 1-(4-(3-(6-(4-isopropyl-4H-1,2,4-triazol-3-yl)pyridin-2-yl)-2-oxoimidazolidin-1-yl)phenyl)azetidine-3-carbonitrile;

1082: 1-(4-(3-(6-(4-isopropyl-4H-1,2,4-triazol-3-yl)pyridin-2-yl)-2-oxoimidazolidin-1-yl)phenyl)-N-methylpiperidine-4-carboxamide;

1097: 1-(4-(3-(6-(4-isopropyl-4H-1,2,4-triazol-3-yl)pyridin-2-yl)-2-oxoimidazolidin-1-yl)phenyl)-N,N-dimethylpiperidine-4-carboxamide.

The compounds of the present invention may be present as their derivatives, analogs, tautomeric forms, stereoisomers, polymorphs, hydrates, solvates, pharmaceutically acceptable salts and compositions, metabolites and prodrugs thereof

The pharmaceutically acceptable salts are prepared by reacting the compound of formula (I) or formula (II(with 1 to 10 equivalents of a base such as sodium hydroxide, sodium methoxide, sodium hydride, potassium t-butoxide, calcium hydroxide, magnesium hydroxide and the like, in solvents like ether, tetrahydrofuran, methanol, t-butanol, dioxane, isopropanol, ethanol etc. Mixture of solvents may also be used. Organic bases such as diethanolamine, α-phenylethylamine, benzylamine, piperidine, morpholine, pyridine, hydroxyethylpyrrolidine, hydroxyethylpiperidine, choline, guanidine, ammonium, substituted ammonium salts and aluminum salts and amino acids such as glycine, alanine, cystine, cysteine, lysine, arginine, phenylalanine etc may be used for the preparation of amino acid salts. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid, salicylic acid, hydroxynaphthoic acid, ascorbic acid, palmitic acid, succinic acid, benzoic acid, benzenesulfonic acid, tartaric acid, oxalic acid and the like in solvents like ethyl acetate, ether, alcohols, acetone, tetrahydrofuran, dioxane etc. Mixture of solvents may also be used.

It should be noted that compounds of the invention may contain groups that may exist in tautomeric forms, and though one form is named, described, displayed and/or claimed herein, all the forms are intended to be inherently included in such name, description, display and/or claim.

The stereoisomers of the compounds forming part of this invention may be prepared by using reactants in their single enantiomeric form, in the process wherever possible or by conducting the reaction in the presence of reagents or catalysts in their single enantiomeric form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid and the like, wherever applicable or by using chiral bases such as brucine, cinchona alkaloids, their derivatives and the like.

Prodrugs of the compounds of formula (I) are also contemplated by this invention. A prodrug is an active or inactive compound that is modified chemically through in-vivo physiological action, such as hydrolysis, metabolism and the like, into a compound of this invention following administration of the prodrug to a patient. The suitability and techniques involved in making, using prodrugs are well known by those skilled in the art.

Various polymorphs of the compounds of the general formula (I), forming part of this invention may be prepared by crystallization of the compounds of formula (I) under different conditions. For example, using different commonly used solvents, or their mixtures for recrystallization; crystallizations at different temperatures; various modes of cooling, ranging from very fast to very slow cooling during crystallizations. Heating or melting the compounds followed by cooling gradually or immediately, one can also obtain polymorphs. The presence of polymorphs may be determined by solid probe NMR spectroscopy, IR spectroscopy, Raman spectroscopy, differential scanning calorimetry and powder X-ray diffraction or other such techniques.

Pharmaceutically acceptable solvates of the compounds of the formula (I) forming part of this invention may be prepared by conventional methods such as dissolving the compounds of the formula (I) in solvents such as water, methanol, ethanol, mixture of solvents such as acetone/water, dioxane/water, N, N-dimethylformamide/water and the like, preferably water and recrystallization by using different crystallization techniques.

The present invention also discloses a process of preparing the compounds of the present invention. The compounds of the present invention can be prepared by the general synthetic schemes 1, presented here below:

General Synthetic Scheme 1:

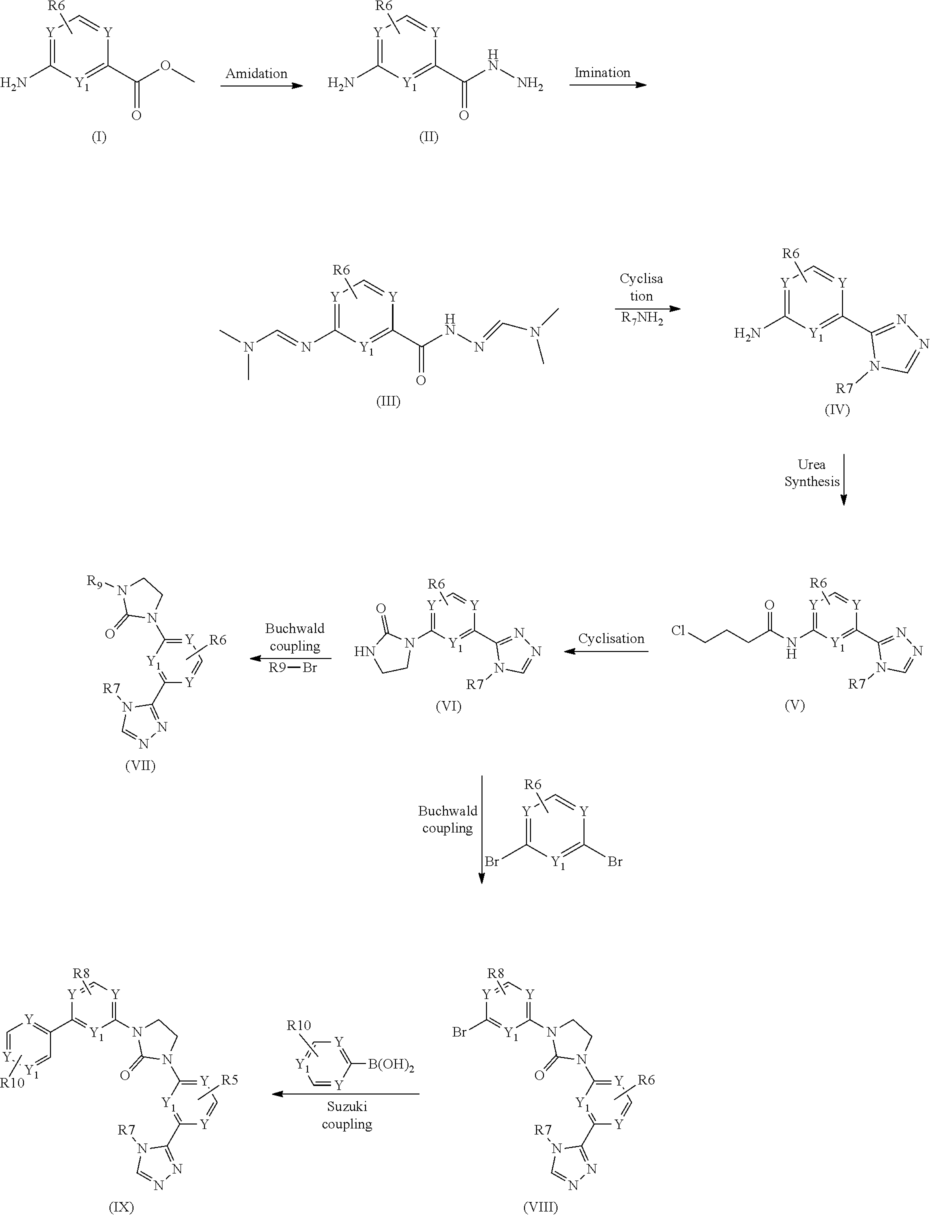

Wherein, Y and Y1 are CH or N;

Wherein, R₆=H, F, CH₃,

Wherein, R₁₀=—CN, —CF₃, —CONH₂, —SO₂CH₃, —CH₃,

Wherein, R₈=H, F, CH₃, OCH3,

Wherein,

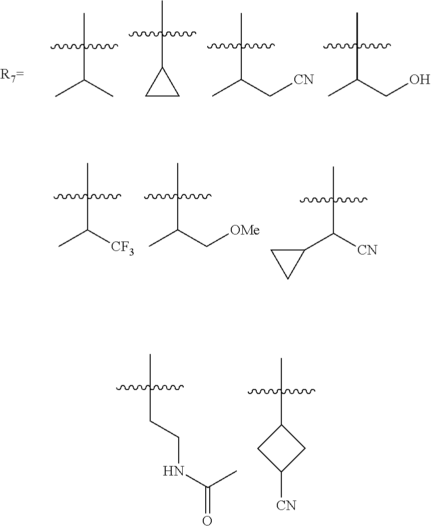

Wherein,

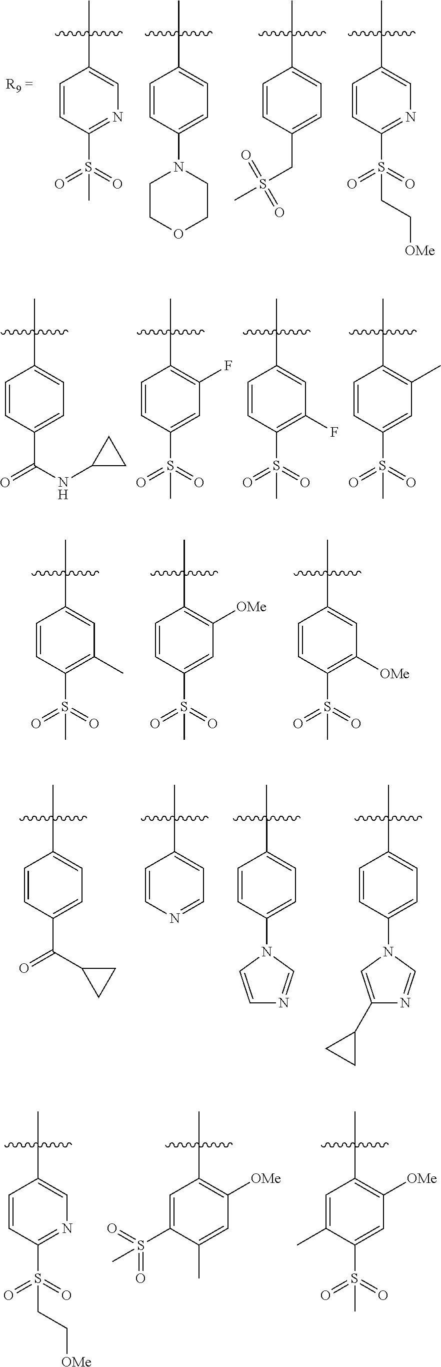

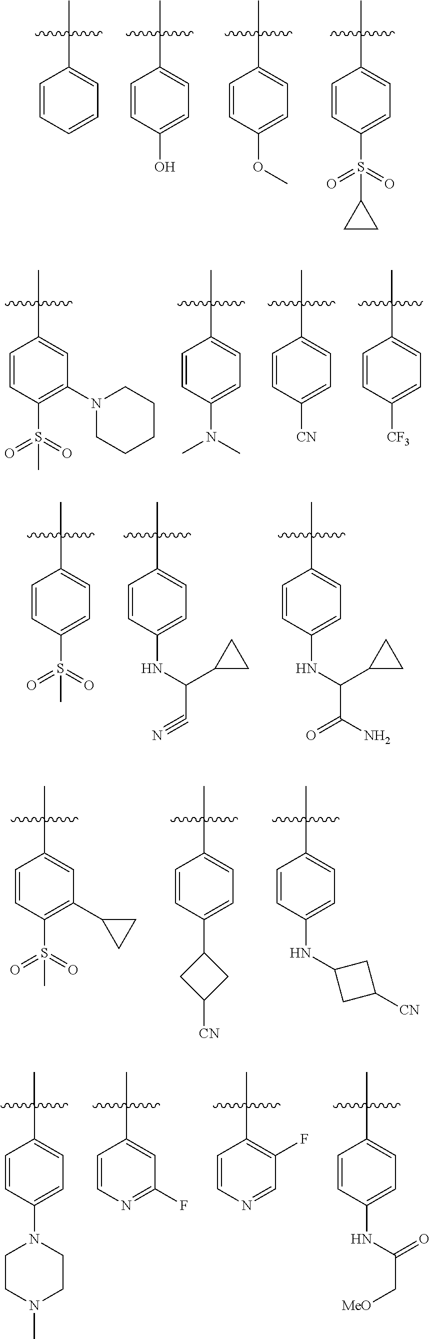

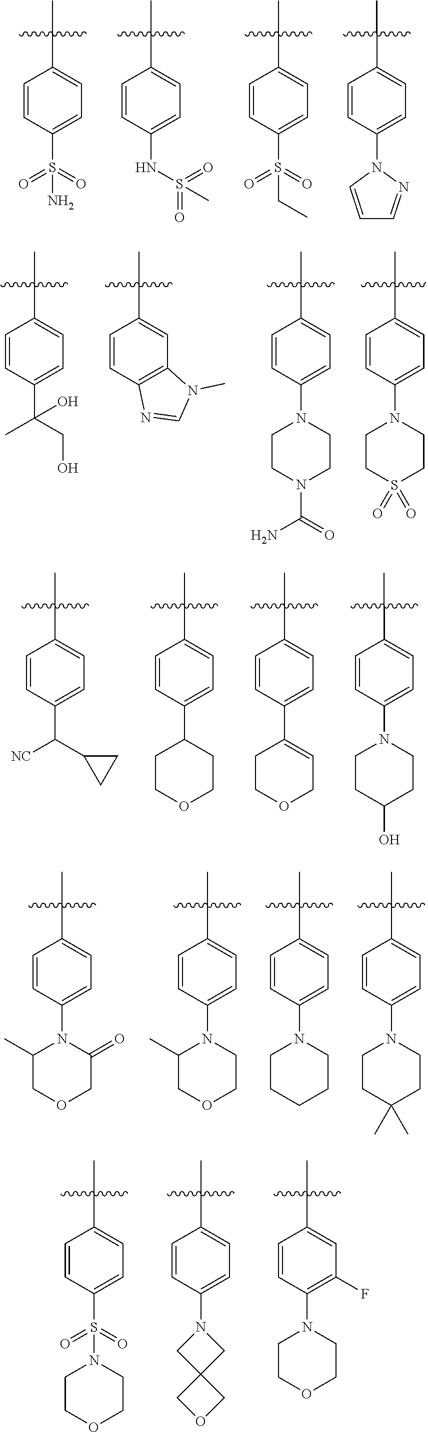

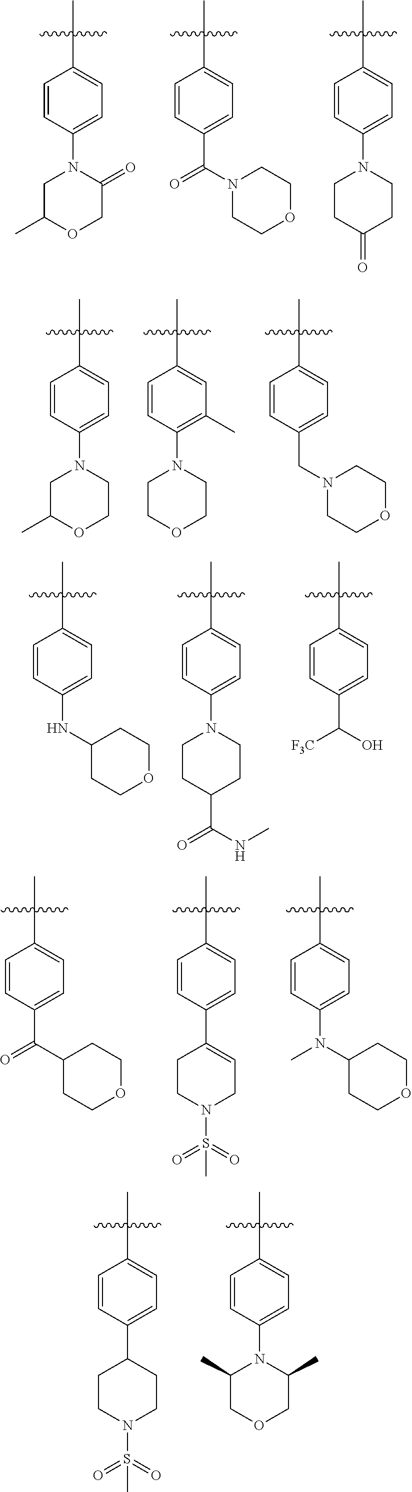

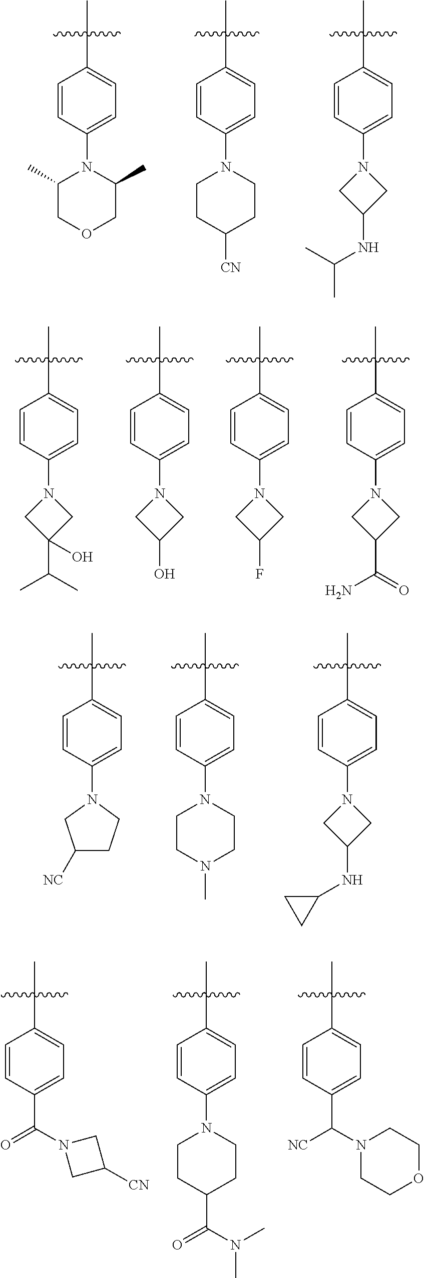

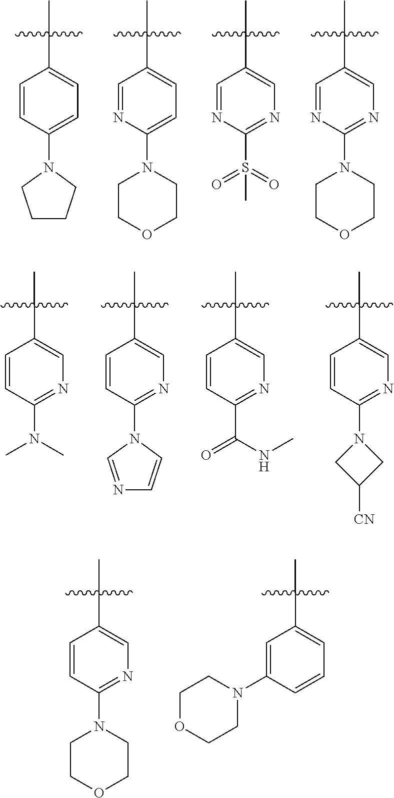

The invention also comprises as another embodiment, a composition comprising the compound of the present invention according to any one of the preceding embodiments together with a pharmaceutically acceptable diluent, excipient, and/or carrier. The compositions will include a conventional pharmaceutical carrier, excipient, and/or diluent and a compound of this disclosure as the/an active agent, and, in addition, can include carriers and adjuvants, etc. The pharmaceutically acceptable compositions will contain about 1% to about 99% by weight of a compound(s) of this disclosure, or a pharmaceutically acceptable salt thereof, and 99% to 1% by weight of a suitable pharmaceutical excipient.

Administration of the compounds of this disclosure, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages.

Solid dosage forms for oral administration include capsules, tablets, pills, powders, and granules. Solid dosage forms, as described above, can be prepared with coatings and shells, such as enteric coatings. Liquid dosage forms for oral administration include pharmaceutically acceptable emulsions, solutions, suspensions, syrups, and elixirs. Compositions for rectal administrations are, for example, suppositories that can be prepared by mixing the compounds of this disclosure with, for example, suitable non-irritating excipients or carriers. They are also be parenteral and administered as sterile powders for reconstitution into sterile injectable solutions or dispersions. Dosage forms for topical administration of a compound of this disclosure include ointments, powders, sprays, and inhalants. Ophthalmic formulations, eye ointments, powders, and solutions are also contemplated for the compounds in this disclosure. Compressed gases can be used to disperse a compound of this disclosure in aerosol form.

The compositions described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg. The amount of active ingredient that may be combined with the carrier materials to produce a single dosage form will vary depending upon the host treated and the particular mode of administration.

In another embodiment, the compounds of the present invention may be utilised in intervention of disorders pertaining to or related to inhibition or intervention of ASK1. Also the compounds of the present invention are expected to intervene innonalcoholic fatty liver disease (NAFLD), obesity, metabolic syndrome, or any type of diabetes, including type 1 and type 2. The compounds of as non-alcoholic fatty liver disease (NAFLD) and non-alcoholic steatohepatitis (NASH). and other fibrotic diseases of the liver; diabetic complications such as macro (ischemic heart disease, cerebrovascular disease and peripheral vascular disease) and micro (cataract, retinopathy nephropathy neuropathy, maculopathy and glaucoma) vascular complication; inflammation such as rheumatoid arthritis and cardiovascular diseases such as atherosclerosis, restenosis, hypertension, vasospasm, and ardiac hypertrophy; and lung disorders and lung fibrosis.

The present invention also provides a pharmaceutical composition, containing one or more of the compounds of the general formula (I) as defined above, their derivatives, analogs, tautomeric forms, stereoisomers, polymorphs, hydrates, metabolites, prodrugs, pharmaceutically acceptable salts, pharmaceutically acceptable solvates in combination with the usual pharmaceutically employed carriers, diluents and the like, useful for the treatment of and/or prophylaxis of liver disorders such as NASH/NAFLD, hepatic fibrosis, liver cirrhosis, steatohepatitis and the like and associated diseases like cardiovascular disease, polycystic ovary syndrome, obstructive apnoea and the like; psoriasis; lung disorders such as lung fibrosis and the like and associated diseases such as lung metastasis and the like; and diabetic complications such as diabetic retinopathy, diabetic nephropathy, diabetic neuropathy, diabetic cataract and the like.

The examples and scheme below depict the general synthetic procedure for the compounds disclosed herein. Synthesis of the compounds of Formulae I disclosed herein, and embodiments thereof, are not limited by these examples and schemes. One skilled in the art will know that other procedures can be used to synthesize the compounds of Formulae I disclosed herein, and that the procedures described in the examples and schemes is only one such procedure. In the descriptions below, one of ordinary skill in the art would recognize that specific reaction conditions, added reagents, solvents, and reaction temperatures can be modified for the synthesis of specific compounds that fall within the scope of this disclosure. All intermediate compounds described below, for which there is no description of how to synthesize such intermediates within these examples below, are commercially available compounds unless otherwise specified.

### EXPERIMENTAL SECTION:

### Example 1005:

### 1-(3-(4-cyclopropyl-1H-imidazol-1-yl)phenyl)-3-(6-(4-isopropyl-4H-1,2,4-triazol-3-yl)pyridin-2-yl)imidazolidin-2-one

### Step 1: Synthesis oft-cyclopropyl-2-oxoethyl 4-methylbenzenesulfonate

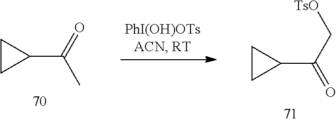

1-cyclopropylethanone (0.62 g, 1.93 mmol) was dissolved in 18 ml acetonitrile. [Hydroxy(tosyloxy)iodo]benzene (0.76 g, 1.93 mmol) was added and the reaction mixture was stirred at reflux for 4 h. The mixture was evaporated and the semi-solid residue was triturated with ether to give the title compound. Purity 92%. Brown solid, yield 0.51 g, 53%.

MS: 255.07 [M+1]

### Step 2: Synthesis of 4-cyclopropyl-1H-imidazole

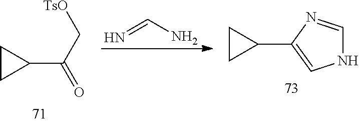

2-cyclopropyl-2-oxoethyl 4-methylbenzenesulfonate (0.400 g, 1.5 mmol) was dissolved in 7 ml ethylene glycol. Formamidine acetate (1.6 g, 15.3 mmol) was added and the reaction mixture was stirred at reflux for 10 h. Reaction was monitored by TLC. On completion, the reaction mass was extracted several times with ethyl acetate. The organic layer was washed with water, brine, dried over sodium sulphate, concentrated under reduced pressure obtained crude which was purified column chromatography; eluent 5% CH₂Cl₂/MeOH to afford 4-cyclopropyl-1H-imidazole as yellowish oil. Yield 0.11 g, 57%.

MS: 109.08 [M+1]

### Step 3: Synthesis of 1-(3-(4-cyclopropyl-1H-imidazol-1-yl)phenyl)-3-(6-(4-isopropyl-4H-1,2,4-triazol-3-yl)pyridin-2-yl)imidazolidin-2-one

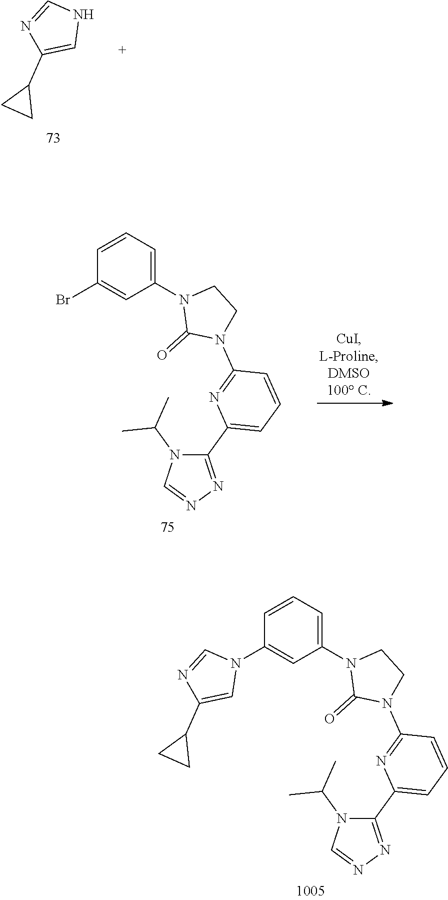 [SMILES: c1nc(C2CC2)c[nH]1 ; CC(C)n1cnnc1-c1cccc(N2CCN(c3cccc(Br)c3)C2=O)n1 ; CC(C)n1cnnc1-c1cccc(N2CCN(c3cccc(-n4cnc(C5CC5)c4)c3)C2=O)n1]

To a stirred solution of 1-(3-bromophenyl)-3-(6-(4-isopropyl-4H-1,2,4-triazol-3-yl)pyridin-2-yl)imidazolidin-2-one (0.05 g, 0.117 mmol) 4-cyclopropyl-1H-imidazole (0.013 g, 0.0117 mmol in DMSO (5 mL) was added CS₂CO₃ (0.115 g, 0.351 mmol), reaction mass was degassed with nitrogen for 30 min. Then, CuI (3.3 mg, 0.017 mmol) and L-Proline (3.91 mg, 0.034 mmol) was added to it, stirred at 100° C. for 16h. Reaction was monitored by TLC. On completion, reaction was diluted with water, extracted with EtOAc. The organic layer was washed with brine, dried over sodium sulphate, concentrated under reduced pressure obtained crude which was purified column chromatography (100-200 Mesh); eluent 3% MeOH/DCM to afford 1-(3-(4-cyclopropyl-1H-imidazol-1-yl)phenyl)-3-(6-(4-isopropyl-4H-1,2,4-triazol-3-yl)pyridin-2-yl)imidazolidin-2-one (0.026g, 52%) as off white solid.

MS: 455.23 [M⁺+1].

Examples 1002,1003,1004,1005,1006,1008,1010,1011,1012,1014,1015,1016,1017,1019, 1020, 1026,1036,1040 and 1104 were synthesized based on above synthetic scheme by changing the substituents at step 4.

### Example 1010

### 1-(6-(4-isopropyl-4H-1,2,4-triazol-3-yl)pyridin-2-yl)-3-(3-(6-(methylsulfonyl)pyridin-3-yl)phenyl)imidazolidin-2-one

### Step-1: Synthesis of 5-bromo-2-(methylthio)pyridine

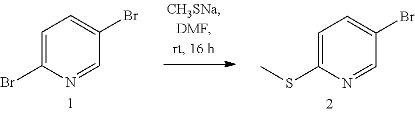

To a stirred solution of 2,5-dibromopyridine (2.0 g, 4.22 mmol) in DMF (20 mL) was added methyl mercaptan sodium salt (1.33 ml g, 4.221 mmol) (21% solution in water) and stirred at room temperature for 16h. Reaction was monitored by TLC. On completion reaction mass was concentrated under reduced pressure. Reaction mixture was extracted with ethyl acetate. The organic layer was washed with water, brine, dried over sodium sulphate, concentrated under reduced pressure obtained crude which was purified column chromatography (100-200) mesh; eluent 3% EtOAc/Hexane to afford 5-bromo-2-(methylthio)pyridine (1.5g, 87.20%) as off white solid.

MS: 203.94[W+1]

### Step-2: Synthesis of 5-(4,4,5,5-tetramethyl-1,3,2-dioxaborolan-2-yl)-2-(methylthio)pyridine

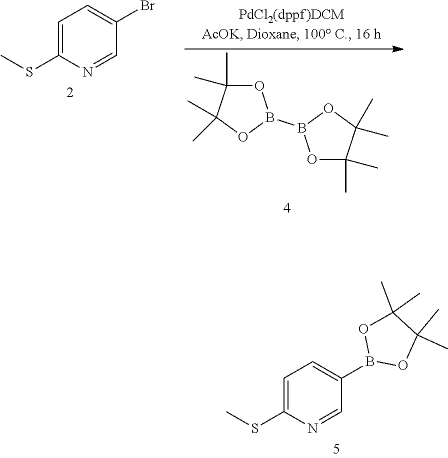

To a stirred solution of 5-bromo-2-(methylthio)pyridine (1.0g, 4.899 mmol) and Bis(pinacolato)diboron (1.86 g, 7.34 mmol) in Dioxane (50 mL) was added AcOK(1.44 g, 14.69 mmol) and reaction mass was purged with nitrogen for 30 min. Then, PdCl2(dppf)DCM (0.399 g, 0.489 mmol) was added to it and stirred at 100° C. for 16h. Reaction was monitored by TLC. On completion reaction mass was concentrated under reduced pressure and extracted with ethyl acetate. The organic layer was washed with water, brine, dried over sodium sulphate, concentrated under reduced pressure obtained crude which was purified column chromatography; eluent 5% EtOAc/Hexane to afford 5-(4,4,5,5-tetramethyl-1,3,2-dioxaborolan-2-yl)-2-(methylthio)pyridine (0.7 g, 56.91%) as off white solid.

MS: 252.1[M⁺+1]

### Step-3: Synthesis of 1-(6-(4-isopropyl-4H-1,2,4-triazol-3-yl)pyridin-2-yl)-3-(3-(6-(methylthio)pyridin-3-yl)phenyl)imidazolidin-2-one

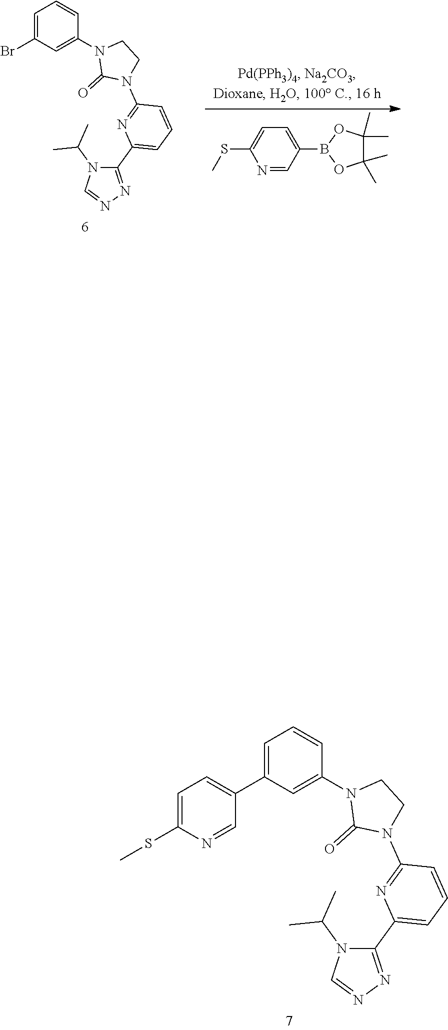

To a stirred solution of 1-(3-bromophenyl)-3-(6-(4-isopropyl-4H-1,2,4-triazol-3-yl)pyridin-2-yl)imidazolidin-2-one (0.03 g, 0.070 mmol) and 5-(4,4,5,5-tetramethyl-1,3,2-dioxaborolan-2-yl)-2-(methylthio)pyridine (0.026 g, 0.105 mmol) in Dioxane (10 mL) was added Na₂CO₃ (0.022 g, 0.211 mmol) in water (2 mL) and reaction mass was degassed with nitrogen for 30 min. Then, Pd(PPh₃)4 (0.008 g, 0.007 mmol) was added to it and stirred at 100° C. for 16h. Reaction was monitored by TLC. On completion reaction was concentrated under reduced pressure obtained crude which was purified by column chromatography (100-200 Mesh); eluent 3% MeOH/HDCM to afford 1-(6-(4-isopropyl-4H-1,2,4-triazol-3-yl)pyridin-2-yl)-3-(3 -(6-(methylthio)pyridin-3 -yl)phenyl)imidazolidin-2-one (0.03g, 90%) as off white solid.

MS: 472.2[M⁺+1]

### Step-4: Synthesis of 1-(6-(4-isopropyl-4H-1,2,4-triazol-3-yl) pyridin-2-yl)-3-(3-(6-(methylsulfonyl) pyridin-3-yl)phenyl)imidazolidin-2-one

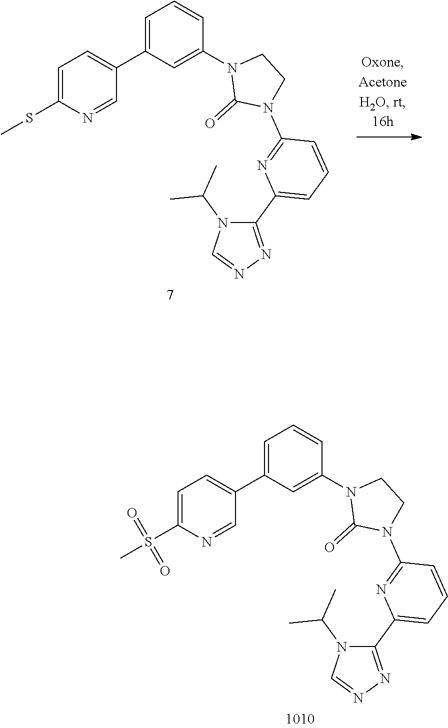 [SMILES: CSc1ccc(-c2cccc(N3CCN(c4cccc(-c5nncn5C(C)C)n4)C3=O)c2)cn1 ; CC(C)n1cnnc1-c1cccc(N2CCN(c3cccc(-c4ccc(S(C)(=O)=O)nc4)c3)C2=O)n1]

To a stirred solution of 1-(6-(4-isopropyl-4H-1,2,4-triazol-3-yl)pyridin-2-yl)-3-(3-(6-(methylthio)pyridin-3-yl)phenyl)imidazolidin-2-one (0.03 g, 0.063 mmol) in acetone (10 mL) was added Oxone (0.058 g, 0.19 mmol) in water (2mL) then, reaction mass was stirred at room temperature for 16h. Reaction was monitored by TLC. On completion reaction was concentrated under reduced pressure and extracted with ethyl acetate. The organic layer was washed with water, brine, dried over sodium sulphate, concentrated under reduced pressure obtained crude which was purified column chromatography (100-200 Mesh); eluent 4% MeOH/DCM to afford 1-(6-(4-isopropyl-4H-1,2,4-triazol-3 -yl)pyridin-2-yl)-3 -(3 -(6-(methylsulfonyl)pyridin-3-yl)phenyl)imidazolidin-2-one (0.0012g, 37%) as off white solid.

MS: 504.17[M⁺+1]

### Example 1017

### 1-(6-(4-isopropyl-4H-1,2,4-triazol-3-yl)pyridin-2-yl)-3-(4′-(trifluoromethyl)-[1,1′-biphenyl]-3-yl)imidazolidin-2-one

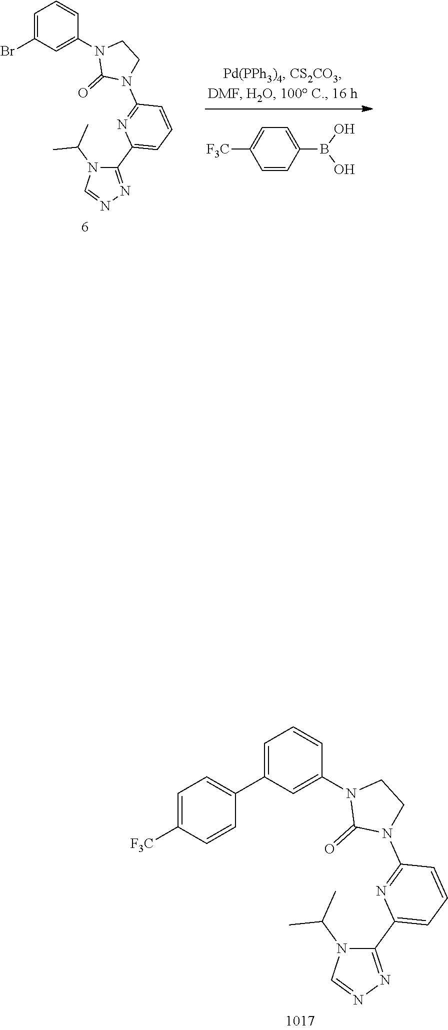 [SMILES: CC(C)n1cnnc1-c1cccc(N2CCN(c3cccc(Br)c3)C2=O)n1 ; OB(O)c1ccc(C(F)(F)F)cc1 ; CC(C)n1cnnc1-c1cccc(N2CCN(c3cccc(-c4ccc(C(F)(F)F)cc4)c3)C2=O)n1]

To a stirred solution of 1-(3-bromophenyl)-3-(6-(4-isopropyl-4H-1,2,4-triazol-3-yl)pyridin-2-yl)imidazolidin-2-one (6, 0.05 g, 0.117 mmol) and 4-(trifluoromethyl)phenylboronic acid (0.033 g, 0.017 mmol in DMF (5 mL) was added CS₂CO₃ (0.11 g, 0.321 mmol), reaction mass was degassed with nitrogen for 30 min. Then, Pd(PPh₃)₄(0.013 g, 0.011 mmol) was added to it , stirred at 100° C. for 16h. Reaction was monitored by TLC. On completion reaction was diluted with water , extracted with EtOAc . The organic layer was washed with brine, dried over sodium sulphate, concentrated under reduced pressure obtained crude which was purified column chromatography (100-200 Mesh); eluent 3% MeOH/DCM to afford 1-(6-(4-isopropyl-4H-1,2,4 -triazol-3 -yl)pyridin-2-yl)-3 -(4-(methylsulfonyl)phenyl)imidazolidin-2-one (0.03g, 52%) as off white solid.

MS: 493.2[M⁺+1]

### Example 1013

### 1-(6-(4-isopropyl-4H-1,2,4-triazol-3-yl)pyridin-2-yl)-3-(4(methylsulfonyl) phenyl)imidazolidin-2- one

### Step-1: Synthesis of (4-bromophenyl)(methyl)sulfane

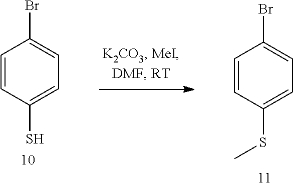

To a stirred solution of 4-bromobenzenethiol (1.0g ,5.31 mmol) in DMF (10 mL) was added K₂CO₃ (1.46 g,10.63 mmol) followed by MeI (1.13 g,7.97mmol) at RT and allowed to stir for 12h at same temperature. Reaction was monitored by TLC. On completion reaction was diluted with water , extracted with EtOAc .The organic layer was washed with brine, dried over sodium sulphate, concentrated under reduced pressure obtained (4-bromophenyl)(methyl)sulfane (0.7 g,70%) as light brown solid.

MS: 203.2 [M⁺+1]

### Step-2: Synthesis of 1-bromo-4-(methylsulfonyl)benzene

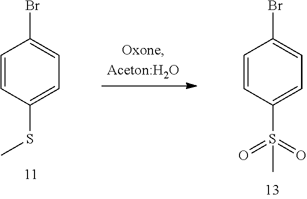

To a stirred solution of (4-bromophenyl)(methyl)sulfane (0.5 g, 2.47 mmol) in acetone (10 mL) was added Oxone(0.76 g, 2.47 mmol) in water (5mL) then, reaction mass was stirred at room temperature for 16h. Reaction was monitored by TLC. On completion reaction was concentrated under reduced pressure and extracted with ethyl acetate. The organic layer was washed with water, brine, dried over sodium sulphate, concentrated under reduced pressure obtained 1-bromo-4-(methylsulfonyl)benzene (0.3g, 51%) as light brown solid.

MS: 235.17[M⁺+1]

### Step-3: Synthesis of 1-(6-(4-isopropyl-4H-1,2,4-triazol-3-yl) pyridin-2-yl)-3-(4-(methylsulfonyl) phenyl) imidazolidin-2-one

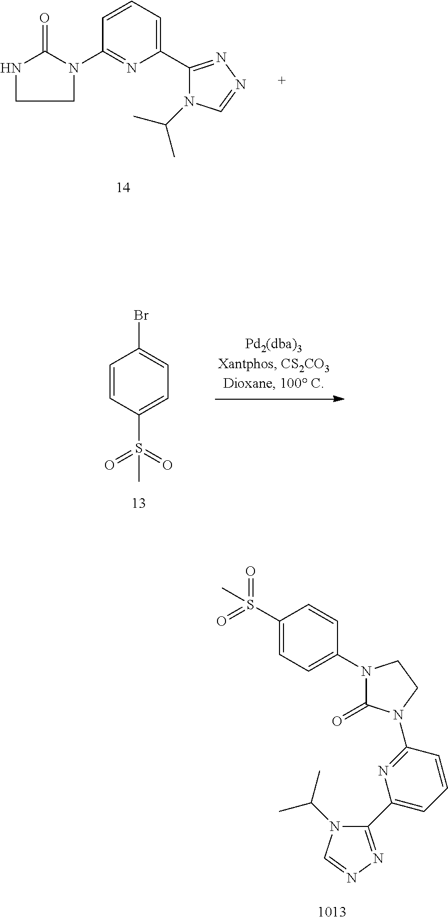 [SMILES: CC(C)n1cnnc1-c1cccc(N2CCNC2=O)n1 ; CS(=O)(=O)c1ccc(Br)cc1 ; CC(C)n1cnnc1-c1cccc(N2CCN(c3ccc(S(C)(=O)=O)cc3)C2=O)n1]

To a stirred solution of 1-(6-(4-isopropyl-4H-1,2,4-triazol-3-yl)pyridin-2-yl)imidazolidin-2-one (0.05 g, 0.183 mmol) and 1-bromo-4-(methylsulfonyl)benzene (0.047 g, 0.202 mmol) in Dioxane (10 mL) was added CS₂CO₃ (0.18 g, 0.551 mmol), reaction mass was degassed with nitrogen for 30 min. Then, Pd₂(dba)₃ (0.017 g, 0.018 mmol) and xantphos (0.021 g, 0.036 mmol) were added to it and stirred at 100° C. for 16h. Reaction was monitored by TLC. On completion reaction was diluted with water , extracted with EtOAc .The organic layer was washed with brine, dried over sodium sulphate, concentrated under reduced pressure obtained crude which was purified column chromatography (100-200 Mesh); eluent 3% MeOH/DCM to afford 1-(6-(4-isopropyl-4H- 1,2,4-triazol-3 -yl)pyridin-2-yl)-3 -(4-(methylsulfonyl)phenyl)imidazolidin-2-one (0.03g, 38%) as off white solid.

MS: 427.2[M⁺+1]

Examples1009,1007,1018,1021,1023,1024,1025,1027,1029,1030,1031,1032,1033,1034,1035, 1037,1038,1039,1041,1042,1044,1045,1046,1047,1048,1049,1050,1051,1052,1053,1054,1055, 1056,1057,1061,1062,1064,1065,1083,1102,1105,1106,1109,1110,1117,1118,1123,1124, 1125 and 1126 were synthesized based on above synthetic scheme by changing the substituents at step 3.

### Example 1025

### 1-(4-(cyclopropylsulfonyl)phenyl)-3-(6-(4-isopropyl-4H-1,2,4-triazol-3-yl)pyridin-2-yl)imidazolidin-2-one

### Step 1: (4-bromophenyl) (3-chloropropyl) sulfane

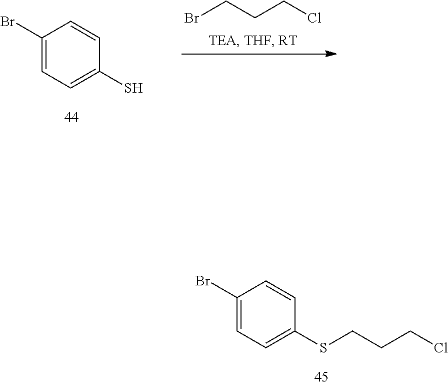 [SMILES: ClCCCBr ; Sc1ccc(Br)cc1 ; ClCCCSc1ccc(Br)cc1]

To a stirred solution of 4-bromobenzenethiol (1.0 g, 5.3 mmol) in THF (20 mL) was added TEA (2.0 g, 18.6 mmol) and stirred at 0° C. for 30 min, than 1-bromo-3-chloropropane (2.0 g, 18.6 mmol) and stir for 3 hr at RT. Reaction was monitored by TLC. On completion reaction mass was concentrated under reduced pressure. Reaction mixture was quenched with H₂O and extracted with ethyl acetate. The organic layer was washed with water, brine, dried over sodium sulphate, concentrated under reduced pressure to obtained crude which was purified by column chromatography; eluent 100% Hexane to afford (4-bromophenyl)(3-chloropropyl)sulfane(0.7 g, 50.0%) as off transparent liquid.

MS: 265[M⁺+1]

### Step 2: 1-(3-chloropropylsulfonyl)-4-bromobenzene

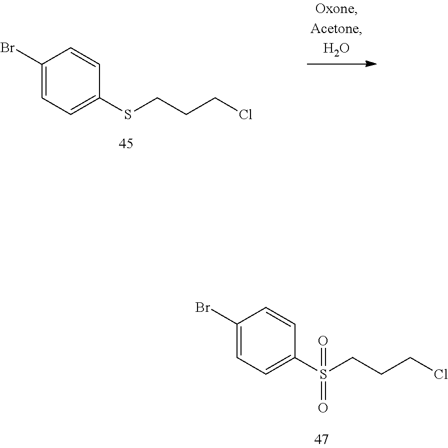 [SMILES: ClCCCSc1ccc(Br)cc1 ; O=S(=O)(CCCCl)c1ccc(Br)cc1]

To a stirred solution of (4-bromophenyl)(3-chloropropyl)sulfane (0.7 g, 2.59 mmol) in Acetone (20 mL) into a RBF at 0° C. for 30 min, than Oxone (1.77 g, 6.65 mmol) in H₂O (20 ml) added to it and stir for 0/N at RT. Reaction was monitored by TLC. On completion reaction mass was concentrated under reduced pressure. Reaction mixture was quenched with H₂O and extracted with ethyl acetate. The organic layer was washed with water, brine, dried over sodium sulphate, concentrated under reduced pressure to obtained crude which was purified by column chromatography; eluent 10% Ethyl acetate/Hexane to afford1-(3-chloropropylsulfonyl)-4-bromobenzene (0.2 g, 78.40%) as off white solid.

MS: 297[M⁺+1]

### Step 3: 1-bromo-4-(cyclopropylsulfonyl) benzene

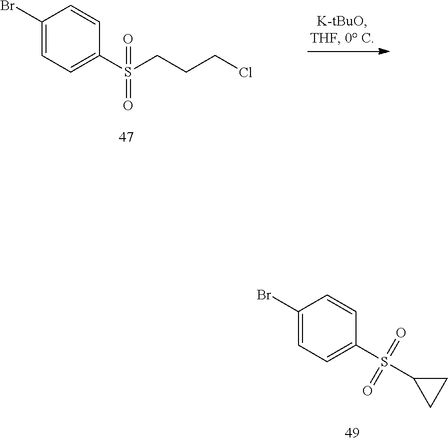 [SMILES: O=S(=O)(CCCCl)c1ccc(Br)cc1 ; O=S(=O)(c1ccc(Br)cc1)C1CC1]

To a stirred solution of 1-(3-chloropropylsulfonyl)-4-bromobenzene (0.100 g, 0.34 mmol) in THF (5 mL) into a vial at 0° C. than added KtBuO (0.06 g, 0.50 mmol) and stir for 5 hr at 50° C. Reaction was monitored by TLC. On completion reaction mass was concentrated under reduced pressure. Reaction mixture was quenched with H₂O and extracted with ethyl acetate. The organic layer was washed with water, brine, dried over sodium sulphate, concentrated under reduced pressure to obtained crude which was purified by column chromatography; eluent 15% Ethyl acetate/Hexane to affordl-bromo-4-(cyclopropylsulfonyl) benzene (0.08 g, 91.95%) as off white solid.

MS: 261[M⁺+1]

### Step 4: 1-(4-(cyclopropylsulfonyl) phenyl)-3-(6-(4-isopropyl-4H-1,2,4-triazol-3-yl) pyridin-2-yl) imidazolidin-2-one

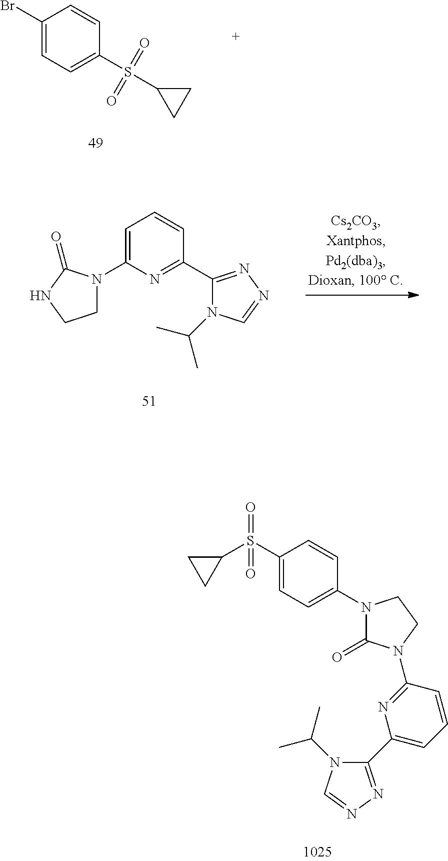 [SMILES: O=S(=O)(c1ccc(Br)cc1)C1CC1 ; CC(C)n1cnnc1-c1cccc(N2CCNC2=O)n1 ; CC(C)n1cnnc1-c1cccc(N2CCN(c3ccc(S(=O)(=O)C4CC4)cc3)C2=O)n1]

To a stirred solution of 1-bromo-4-(cyclopropylsulfonyl) benzene (0.070 g, 0.27 mmol), 1-(6-(4-isopropyl-4H-1,2,4-triazol-3-yl) pyridin-2-yl) imidazolidin-2-one (0.073 g, 0.27 mmol) and CS₂CO₃ (0.26 g, 0.81 mmol) in Dioxane (5 mL) and reaction mass was purged with nitrogen for 30 min. Then, Xantphos (0.031 g, 0.054 mmol) and Pd₂dba₃ (0.025 g, 0.027 mmol) was added to it and stirred at 100° C. for 2 hr. Reaction was monitored by TLC. On completion reaction was quenched with water and extracted with ethyl acetate and dried over sodium sulphate, organic layer evaporate under reduced pressure to obtained crude which was purified by column chromatography (100-200 Mesh); eluent 4.5% MeOH/DCM to afford 1-(4-(cyclopropylsulfonyl) phenyl)-3-(6-(4-isopropyl-4H-1,2,4-triazol-3-yl) pyridin-2-yl) imidazolidin-2-one(0.04 g, 46.5%) as white solid.

MS: 453.10 [M⁺+1]

### Example 1037

### 1-(4-(cyclopropanecarbonyl)phenyl)-3-(6-(4-isopropyl-4H-1,2,4-triazol-3-yl)pyridin-2-yl)imidazolidin-2-one

### Step-1: (4-bromophenyl) (cyclopropyl) methanone

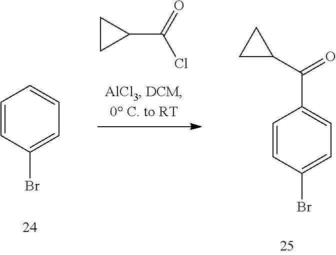 [SMILES: Brc1ccccc1 ; O=C(Cl)C1CC1 ; O=C(c1ccc(Br)cc1)C1CC1]

To a stirred solution of bromo benzene (2.0 g, 1.27 mmol) in DCM (20 mL) was added AlCl₃(2.03 g, 1.52 mmol) and stirred at 0° C. for 30 min, than cyclopropanecarbonyl chloride (1.31 ml, 1.46 mmol) and stir for 3 hr at RT. Reaction was monitored by TLC. On completion reaction mass was concentrated under reduced pressure. Reaction mixture was quenched with H₂O and extracted with ethyl acetate. The organic layer was washed with water, brine, dried over sodium sulphate, concentrated under reduced pressure to obtained crude which was purified by column chromatography; eluent 10% EtOAc/Hexane to afford (4-bromophenyl)(cyclopropyl)methanone (1.0 g, 35.0%) as off transparent liquid.

MS: 225, 227[M⁺+1, M⁺+2]

### Step-2: 1-(4-(Cyclopropyl) phenyl)-3-(6-(4-isopropyl-4H-1,2,4-triazol-3-yl) pyridin-2-yl) imidazolidin-2-one

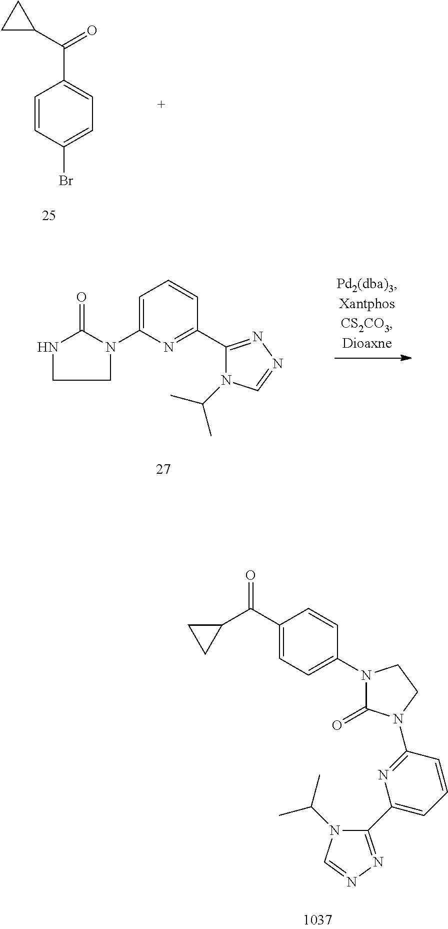 [SMILES: O=C(c1ccc(Br)cc1)C1CC1 ; CC(C)n1cnnc1-c1cccc(N2CCNC2=O)n1 ; CC(C)n1cnnc1-c1cccc(N2CCN(c3ccc(C(=O)C4CC4)cc3)C2=O)n1]

To a stirred solution of (4-bromophenyl) (cyclopropyl) methanone (0.04 g, 0.172 mmol), 1-(6-(4-isopropyl-4H-1,2,4-triazol-3-yl) pyridin-2-yl) imidazolidin-2-one (0.046 g, 0.172 mmol) and CS₂CO₃ (0.164 g, 0.510 mmol) in Dioxane (5 mL) and reaction mass was purged with nitrogen for 30 min. Then, Xantphos (0.02 g, 0.034 mmol) and Pd₂dba₃ (0.014 g, 0.017 mmol) was added to it and stirred at 100° C. for 2 hr. Reaction was monitored by TLC. On completion reaction was quenched with water and extracted with ethyl acetate and dried over sodium sulphate, organic layer evaporate under reduced pressure to obtained crude which was purified by column chromatography (100-200 Mesh); eluent 4.5% MeOH/DCM to afford 1-(4-(Cyclopropyl) phenyl)-3-(6-(4-isopropyl-4H-1,2,4-triazol-3-yl) pyridin-2-yl) imidazolidin-2-one (0.010g, 13.51%) as off white solid.

MS: 417.2[M⁺+1]

### Example 1043

### 1-(6-(4-isopropyl-4H-1,2,4-triazol-3-yl)pyridin-2-yl)-3-(4-morpholinophenyl)imidazolidin-2-one

### Step 1: Synthesis of 4-(4-nitrophenyl) morpholine

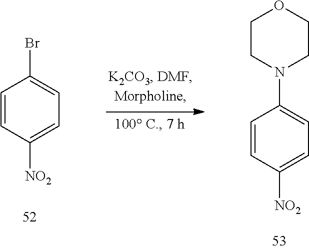 [SMILES: O=[N+]([O-])c1ccc(Br)cc1 ; O=[N+]([O-])c1ccc(N2CCOCC2)cc1]

A mixture of 1-bromo-4-nitrobenzene (1.0 g, 4.8 mmol), morpholine (1.34 g, 18.5 mmol) and potassium carbonate (2.6 g, 18.5 mmol) in DMF (8 ml) were stirred and heated to 100° C. for 7 hours. The resultant reaction solution was allowed to cool to room temperature. The mixture solution was extracted with EtOAc. The organic layer was collected, washed with brine, dried over Na₂SO_{4,} filtered, and dried under reduced pressure. The crude product was purified with silica-gel column chromatography (60% EtOAc/hexane) to give the desired product of 4-(4-nitrophenyl)morpholine as a yellow solid, 0.480 g, 47% yield.

MS: 209.09 [M+1]

### Step 2: Synthesis of 4-morpholinobenzenamine

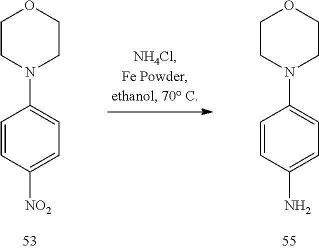 [SMILES: O=[N+]([O-])c1ccc(N2CCOCC2)cc1 ; Nc1ccc(N2CCOCC2)cc1]

To a solution of intermediate 4-(4- nitro phenyl) morpholine (0.30 g, 1.2 mmol) in ethanol (4 ml) and water (30 ml), iron (0.34 g, 6.2 mmol) and ammonium chloride (1.3 g, 2.5 mmol) were added and refluxed for 3h. The reaction mixture was filtered through celite, washed with ethyl acetate. The organic layer was dried over sodium sulphate and concentrated under reduced pressure to afford the title compound was obtained, which was used without purification in the next step. Brown solid, yield: 0.25 g, 96%.

MS: 209.09 [M+1]

### Step 3: Synthesis of 4-(4-bromophenyl) morpholine

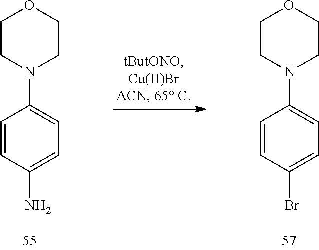 [SMILES: Nc1ccc(N2CCOCC2)cc1 ; Brc1ccc(N2CCOCC2)cc1]

To a solution of copper(II) bromide (0.20 g, 1.12 mmol) in acetonitrile (6 mL) was added tert-butyl nitrite (0.231 mL, 35.3 mmol). The mixture was stirred for 5 minutes. Then a solution of 4-morpholinobenzenamine (6.1 g, 23.53 mmol) in acetonitrile (10 mL) was added dropwise. Stirring was continued at room temperature for 2 hours. The reaction is exothermic. After concentrating the solvent, the residue was diluted with ethyl acetate (50 mL), washed with water (2×10 mL), and then dried over anhydrous sodium sulphate. The dried solution was concentrated in vacuo, and the residue was purified by chromatography on silica gel with petroleum/ethyl acetate to give 4-(4-bromophenyl) morpholine yield: 180 mg, 67%.

MS: 242.01 [M+1].

### Step 4: Synthesis of 6-aminopyridine-2-carbohydrazide

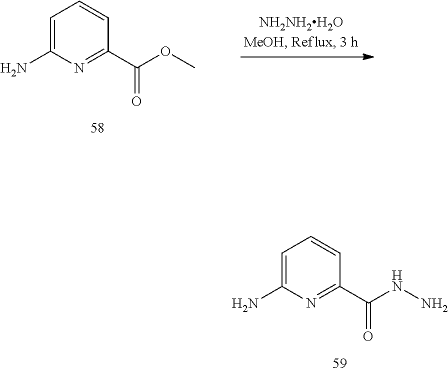

To a solution of methyl 6-aminopicolinate (4.32 g, 0.0284 mol) in MeOH (5 L) was added NH₂NH_{2.}H₂O (2.84 g, 0.0568 mol, 2.0 eq.). The reaction mixture was heated under reflux for 3 hr and then cooled to room temperature. The precipitate formed in the mixture was collected by filtration, washed with EA (1 Lx2) and then dried in vacuo to give compound A (4.05 g, 94% yield) as white solid. MS: 153.07 [M+1].

### Step 5: Synthesis of N′-((dimethyl amino) methylene)-6-(formamido) pyridine-2-carbohydrazide

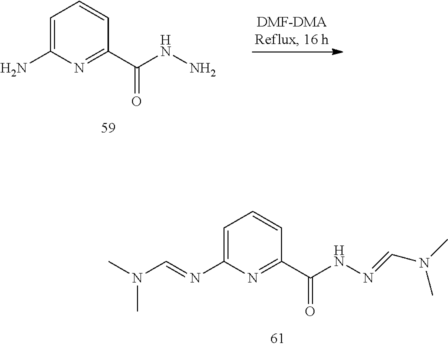

A mixture of compound 59 (4.05 g, 0.0266 mol) in dimethylformamide-dimethylacetal (DMF-DMA) (35 mL) was heated under reflux for 18 hr, cooled to room temperature and then concentrated under reduced pressure. The residue was taken up in EA (50 mL) and heated at 50° C. for 20 min. After being cooled to room temperature, the solid was collected by filtration and dried in vacuo to give compound B (57.2 g, 82% yield) as off white solid.

MS: 263.15 [M+1]

### Step 6: Synthesis of 6-(4-isopropyl-4H-1,2,4-triazol-3-yl)pyridin-2-amine

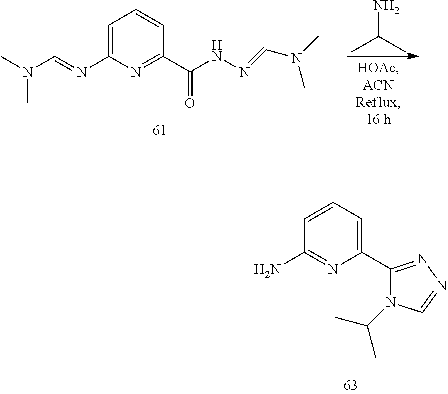

To a solution of compound 61 (5.72 g, 0.0218 mol) in a mixture of CH3CN-AcOH (36 mL, 4:1) was added propan-2-amine (6.46 g, 5.0 eq.). The resulting mixture was heated under reflux for 24 h and then cooled to room temperature, and the solvent was removed under reduced pressure. The residue was dissolved in water (28 mL) and 1N aqueous NaOH was added to a pH of 8.0. The precipitate was collected by filtration and the filtrate was extracted with EA (100 mLx3). The combined organic layers were dried over anhydrous Na₂SO₄, and then concentrated to a volume of 50 mL. To this mixture at 0° C. was slowly added Petroleum Ether (40 mL) and the resulting suspension was filtered. The combined solid was re-crystallized from EA-PE to give compound 63 (2.53 g, 57% yield) as off-white solid.

MS: 204.117 [M+1]

### Step 7: Synthesis of 1-(2-chloroethyl)-3-(6-(4-isopropyl-4H-1,2,4-triazol-3-yl)pyridin-2-yl)urea

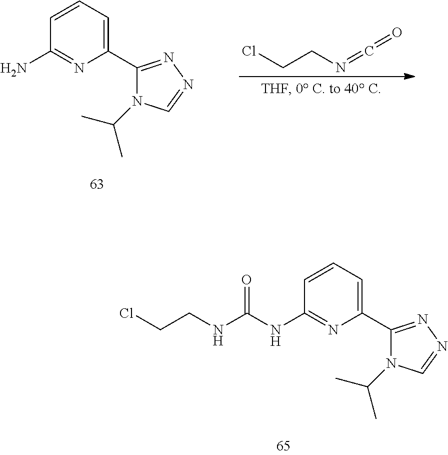 [SMILES: CC(C)n1cnnc1-c1cccc(N)n1 ; O=C=NCCCl ; CC(C)n1cnnc1-c1cccc(NC(=O)NCCCl)n1]

2-Chloroethylisocyanate (1.5 g, 1.5 mol) was added dropwise to a cold solution (ice bath) of 6-(4-isopropyl-4H-1,2,4-triazol-3-yl)pyridin-2-amine (2.0 g, 0.980 mol) in dry THF (30 mL). The ice bath was then removed and the reaction mixture was stirred at room temperature for 24 h. After completion of the reaction, the solvent was evaporated under reduced pressure to give white solid, which was triturated twice in a mixture of cold hexanes/ether 10:1 to give the desired compound (1.8 g, 60% yield) as white solid. The solid compound was pure enough to be used further.

MS: 309.12 [M+1]

### Step 8: Synthesis of 1-(6-(4-isopropyl-4H-1,2,4-triazol-3-yl)pyridin-2-yl)imidazolidin-2-one

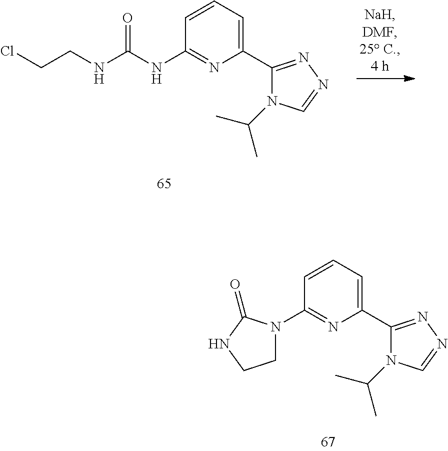 [SMILES: CC(C)n1cnnc1-c1cccc(NC(=O)NCCCl)n1 ; CC(C)n1cnnc1-c1cccc(N2CCNC2=O)n1]

Sodium hydride (480 mg, 9.6 mmol) was added slowly to a cold solution (at 0° C.) of compound 65 (1.5 g, 4.85 mmol) in tetrahydrofuran under dry nitrogen atmosphere. The ice bath was then removed after 30 min and the reaction mixture was stirred at room temperature for 5 h. The reaction was quenched at 0° C. with water and diluted with ethyl acetate. The organic layer was washed with water, brine and dried over anhydrous sodium sulfate, filtered, and concentrated in vacuo to provide the crude product which was purified by coloumn chromatography to give pure 6-(4-isopropyl-4H-1,2,4-triazol-3-yl)pyridin-2-yl)imidazolidin-2-one as white solid. Yield: 1.1 g; 84%.

MS: 273.15 [M+1]

### Step 9: 1-(6-(4-isopropyl-4H-1,2,4-triazol-3-yl)pyridin-2-yl)-3-(4-morpholinophenyl)imidazolidin-2-one

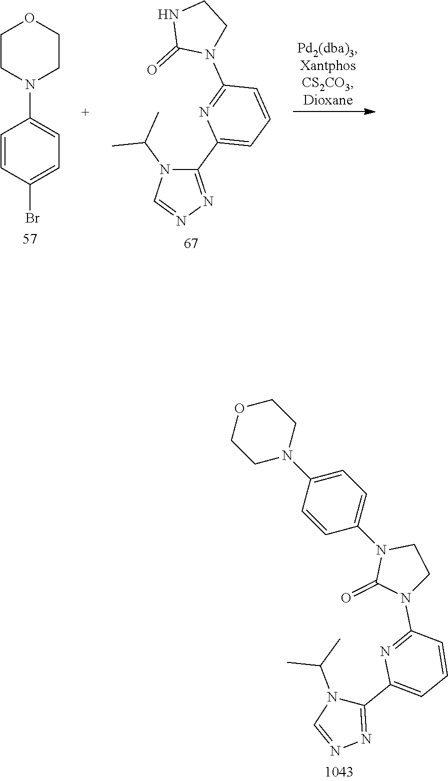 [SMILES: Brc1ccc(N2CCOCC2)cc1 ; CC(C)n1cnnc1-c1cccc(N2CCNC2=O)n1 ; CC(C)n1cnnc1-c1cccc(N2CCN(c3ccc(N4CCOCC4)cc3)C2=O)n1]

A sealed tube was charged with 4-(4-bromophenyl)morpholine (70 mg, 0.28 mmol), 6-(4-isopropyl-4H-1,2,4-triazol-3-yl)pyridin-2-amine (74 mg, 0.28 mmol) and dioxane. The solution was rigorously degassed by nitrogen. To this degassed solution tris(dibenzylideneacetone)dipalladium(0) (25 mg, 0.028 mmol), xantphos (24 mg, 0.0024 mmol), CS₂CO₃ (275 mg, 0.84 mmol) was added and capped with a Teflon screw cap. The mixture was stirred at 100° C. for 4h, cooled to room temperature and diluted with ethyl acetate. The solution was filtered to remove the insoluble and evaporated under reduced pressure. The compound was purified by column chromatography by using silica gel (CH₂Cl₂/MeOH) to give pure 1-(6-(4-isopropyl-4H-1,2,4-triazol-3-yl)pyridin-2-yl)-3-(4-morpholinophenyl)imidazolidin-2-one as white solid. Yield: 31 mg; 24%.

MS: 434.23 [M+1]

Examples1058,1059,1060,1063,1066,1067,1068,1069,1070,1071,1072,1074,1075,1076,1077, 1078,1079,1080,1081,1082,1084,1085,1086,1087,1088,1089,1090,1091,1092,1093,1094,1095, 1096,1097,1098,1100,1101,1103,1107,1108,1111,1112,1113,1114,1115,1116,1119,1120, 1121,1122,1127,1128,1129,1130,1131,1132,1133,1134,1135,1136,1137,1138,1139 and 1140 were synthesized based on above synthetic scheme by changing reactants at step 9 while keeping 6-(4-isopropyl-4H-1,2,4-triazol-3-yl)pyridin-2-amine constant synthesized above at step 8.

### Example 1046

### 1-(2-(cyclopropylamino)benzo[d]thiazol-6-yl)-3-(6-(4-isopropyl-4H-1,2,4-triazol-3-yl)pyridin-2-yl)imidazolidin-2-one

### Step 1: Synthesis of 6-bromobenzo[d]thiazol-2-amine

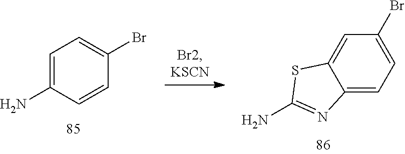 [SMILES: Nc1ccc(Br)cc1 ; Nc1nc2ccc(Br)cc2s1]

A solution of 4-bromoaniline (0.500 g, 2.91 mmol) and potassium thiocyanate (1.13 g, 11.6 mmol) in AcOH (10 mL) was stirred at 20° C. for 10 min. Bromine (150 μL, 2.91 mmol) was added over 20 min to the above solution. The reaction mixture was stirred further at 25° C. for 90 min. On completion of reaction following a TLC examination, the reaction mixture was poured into a solution of NH₄OH and extracted with EtOAc. The organic phase was dried over anhydrous Na₂SO₄ and the solvent was evaporated under vacuo. The crude product obtained was purified by flash chromatography (Hex:AcOEt 1:1) to afford 6-bromobenzo[d]thiazol-2-amine as pale yellow solid (2.67 g, yield 79%). MS: 228.94 [M⁺+1].

### Step 2: Synthesis of 2,6-dibromobenzo[d]thiazole

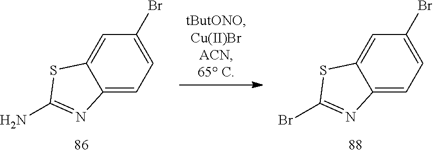 [SMILES: Nc1nc2ccc(Br)cc2s1 ; Brc1ccc2nc(Br)sc2c1]

6- bromobenzo[d]thiazol-2-amine (2.50 g; 14.1 mmol) was dissolved in 70 mL acetonitrile. t-Butylnitrite (1.7 mL; 14 mmol) was added slowly. Cu(II)Br (2.5 g; 17.6 mmol) was added portion-wise through a funnel. The reaction was monitored by HPLC. After 3 hours, ethyl acetate (250 mL) was added, and the mixture was filtered through celite. The organic layer was washed with brine twice (150 mL each). The organic layer was dried over MgSO₄. After filtration, the solvent was removed. The residual was dissolved in 50 mL CH₂Cl₂, and 2 g of silica gel was added. After drying, the silica gel loaded on a silica gel cake in hexane. The product was eluted with 5% ethyl acetate and 95% hexane. A yellowish band was collected. The solvent was removed to afford the product (2.4 g, 77% yield). MS: 291.84 [M⁺+1].

### Step 3: Synthesis of 6-bromo-N-cyclopropylbenzo[d]thiazol-2-amine

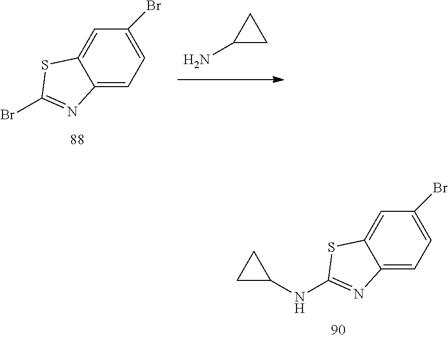

2,6-dibromobenzo[d]thiazole (1.0 g, 3.4 mmol) was suspended in 1,4-dioxane (10 ml), and added cyclopropylamine (3.6 ml, 51.0 mmol). The resultant mixture is stirred under nitrogen for 6 hours and cool to room temperature. On completion of reaction following a TLC examination, the reaction mixture was extracted with ethyl acetate and washed with brine. The solvent was removed. The crude product obtained was purified by flash chromatography (Hex:AcOEt 1:1) to afford 6-bromo-N-cyclopropylbenzo[d]thiazol-2-amine (0.630 mg, 68% yield) as pale yellow solid.

MS: 268.97 [M⁺+1].

### Step 4: Synthesis of tert-butyl 6-bromobenzo[d]thiazol-2-ylcyclopropylcarbamate

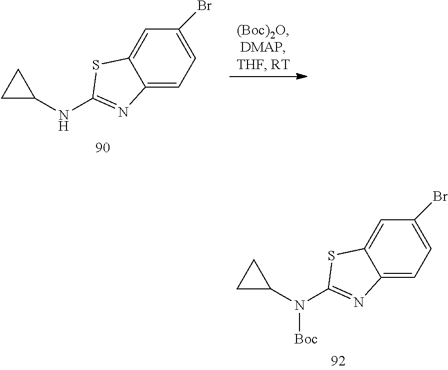 [SMILES: Brc1ccc2nc(NC3CC3)sc2c1 ; CC(C)(C)OC(=O)N(c1nc2ccc(Br)cc2s1)C1CC1]

To a solution of 6-bromo-N-cyclopropylbenzo[d]thiazol-2-amine (0.30 g, 1.10 mmol) in tetrahydrofuran (10 mL) was added di-tert-butyl dicarbonate (0.37 g, 1.60 mmol), DIPEA (2.06 mg, 1.60 mmol) and 4-(dimethylamino)pyridine (19 mg, 0.16 mmol). The resulting mixture was stirred for 4 h at room temperature and diluted with EtOAc. The organic layer was washed with saturated aqueous NaHCO₃ solution and brine. The organic layer was dried (MgSO₄) and concentrated to receive a solid. The solid was suspended in EtOAc and filtered to receive the title compound (0.36 mg, 86% yield).

MS: 369.02 [M⁺+1].

### Step 5: Synthesis of 1-(2-aminobenzo[d]thiazol-6-yl)-3-(6-(4-isopropyl-4H-1,2,4-triazol-3-yl)pyridin-2-yl)imidazolidin-2-one

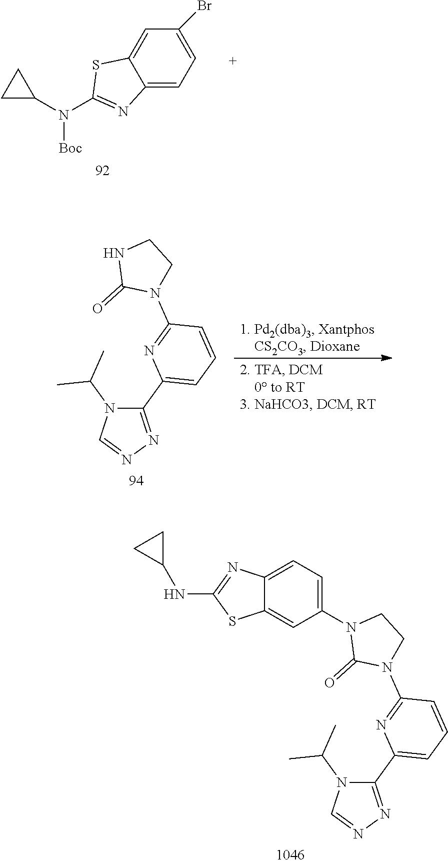 [SMILES: CC(C)(C)OC(=O)N(c1nc2ccc(Br)cc2s1)C1CC1 ; CC(C)n1cnnc1-c1cccc(N2CCNC2=O)n1 ; CC(C)n1cnnc1-c1cccc(N2CCN(c3ccc4nc(NC5CC5)sc4c3)C2=O)n1]

A sealed tube was charged with tert-butyl 6-bromobenzo[d]thiazol-2-ylcyclopropylcarbamate (0.05 g, 0.137 mmol), 6-(4-isopropyl-4H-1,2,4-triazol-3-yl)pyridin-2-amine (0.041 mg, 0.15 mmol) and dioxane . The solution was rigorously degassed by nitrogen. To this degassed solution tris(dibenzylideneacetone)dipalladium(0) (0.013 g, 0.013 mmol), xantphos (0.012 g, 0.019 mmol), CS₂CO₃ (0.090 g, 0.274 mmol) was added and capped with a Teflon screw cap. The mixture was stirred at 100° C. for 4h. On completion, the reaction was diluted with water, extracted with EtOAc. The organic layer was washed with brine, dried over sodium sulphate, concentrated under reduced pressure obtained crude which was purified column chromatography (100-200 Mesh); eluent 4% MeOH/DCM to afford1-(2-aminobenzo[d]thiazol-6-yl)-3 -(6-(4-isopropyl-4H-1,2,4-triazol-3 -yl)pyridin-2-yl)imidazolidin-2-one (0.028 g, 49% yield) as off white solid.

The pure product 20 mg was then dissolved in CH₂C12 and cooled at 0° C. To this stirred solution 0.3mL TFA was added dropwise. The reaction mixture was then allowed to reach at RT and stirred at this temperature for another lh. After completion of the reaction, the reaction mixture was poured into saturated bicarbonate solution and extracted with DCM to afford the title compound (21 mg, 84% yield).

MS: 422.16 [M⁺+1].

### Example 1047

### 1-(6-(4-isopropyl-4H-1,2,4-triazol-3-yl)pyridin-2-yl)-3-(4-((methylsulfonyl) methyl) phenyl)imidazolidin-2-one

### Step 1: Synthesis of (4-bromophenyl) methanol

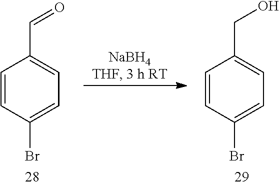 [SMILES: O=Cc1ccc(Br)cc1 ; OCc1ccc(Br)cc1]

To a solution of 4-bromobenzaldehyde (0.5 g, 0.0284 mol) in THF (5 mL) was added NaBH₄(2.84 g, 0.0568 mol) at 0° C. The reaction mixture was stirred for 3 hr at RT. On completion reaction was quenched at 0° C. with water and diluted with ethyl acetate. The organic layer was washed with water, brine and dried over anhydrous sodium sulfate, filtered, and concentrated in vacuo to provide compound (4.05 g, 94% yield) as white solid.

MS: 187.17[M⁺+1]

### Step 2: Synthesis of 1-bromo-4-(bromomethyl) benzene

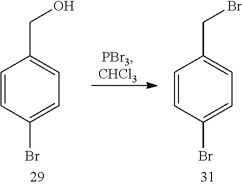 [SMILES: OCc1ccc(Br)cc1 ; BrCc1ccc(Br)cc1]

To a solution of (4-bromophenyl) methanol (0.5 g, 0.0284 mol) in CHCl₃ (5 mL) was added PBr₃ (2.84 g, 0.0568 mol) at 0° C. The reaction mixture was stirred for 1 hr at RT. On completion reaction was quenched at 0° C. with water and diluted with ethyl acetate. The organic layer was washed with water, brine and dried over anhydrous sodium sulfate, filtered, and concentrated in vacuo to provide compound 31 (4.05 g, 94% yield) as white solid.

MS: 249.1[M⁺+1]

### Step 3: Synthesis of 1-bromo-4-((methylsulfonyl) methyl) benzene.

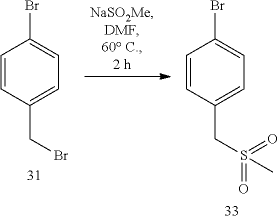

To a solution of 1-bromo-4-(bromomethyl)benzene (0.5 g, 0.0284 mol) in DMF (5 mL) was added Sodium methane sulfonate (2.84 g, 0.0568 mol) at room temperature. The reaction mixture was stirred for 2 hr at 60° C. On completion reaction was quenched with water and diluted with ethyl acetate. The organic layer was washed with water, brine and dried over anhydrous sodium sulfate, filtered, and concentrated in vacuo to provide 1-bromo-4-((methylsulfonyl)methyl)benzene (4.05 g, 94% yield) as white solid.

MS: 249.21[M⁺+1]

### Step-4: Synthesis of 1-(6-(4-isopropyl-4H-1,2,4-triazol-3-yl) pyridin-2-yl) -3-(4-((methylsulfonyl)methyl) phenyl) imidazolidin-2-one.

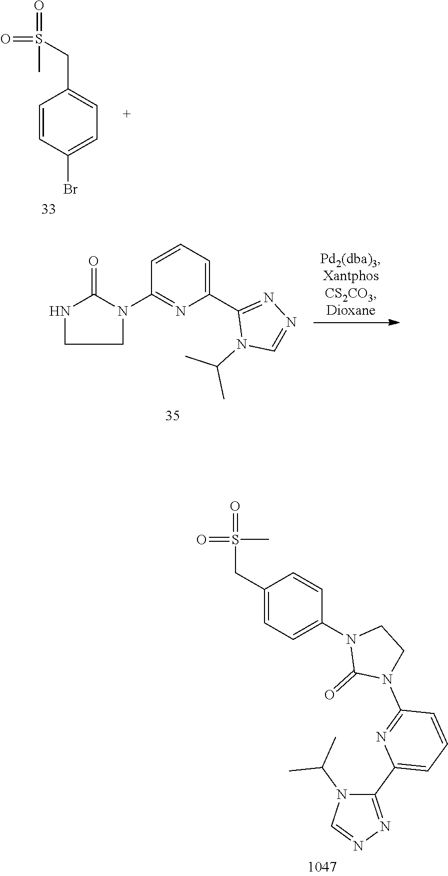 [SMILES: CS(=O)(=O)Cc1ccc(Br)cc1 ; CC(C)n1cnnc1-c1cccc(N2CCNC2=O)n1 ; CC(C)n1cnnc1-c1cccc(N2CCN(c3ccc(CS(C)(=O)=O)cc3)C2=O)n1]

To a stirred solution of 1-bromo-4-((methylsulfonyl) methyl) benzene (0.04 g, 0.160 mmol), 1-(6-(4-isopropyl-4H-1,2,4-triazol-3-yl) pyridin-2-yl) imidazolidin-2-one (0.046 g, 0.172 mmol) and CS₂CO₃ (0.164g, 0.510 mmol) in Dioxane (5 mL) and reaction mass was purged with nitrogen for 30 min. Then, Xantphos (0.02 g, 0.034 mmol) and Pd₂dba₃ (0.014 g, 0.017 mmol) was added to it and stirred at 100° C. for 2 hr. Reaction was monitored by TLC. On completion reaction was quenched with water and extracted with ethyl acetate and dried over sodium sulphate, organic layer evaporate under reduced pressure to obtained crude which was purified by column chromatography (100-200 Mesh); eluent 4.5% MeOH/DCM to afford 1-(6-(4-isopropyl-4H- 1,2 ,4-triazol-3 -yl)pyridin-2-yl)-3 -(4-((methylsulfonyl)methyl)phenyl)imidazolidin-2-one(0.020g, 26.5%) as off white solid.

MS: 441.16[M⁺+1].

### Example 1050

### 1-(4-(2-methoxyethylsulfonyl)phenyl)-3-(6-(4-isopropyl-4H-1,2,4-triazol-3-yl)pyridin-2-yl)imidazolidin-2-one

### Step-1: Synthesis of (2-bromoethyl)(4-bromophenyl)sulfane

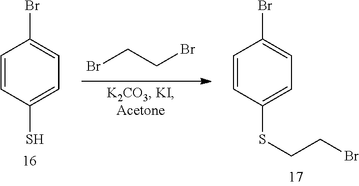

To a stirred solution of 4-bromobenzenethiol (0.6g, 3.17mmol) was added K₂CO₃ (0.65 g, 4.76 mmol) followed by 1,2 dibromo ethane (1.18 g, 6.34 mmol) and KI(0.0052g, 0.031 mmol) at RT and allowed to stir for 12 h at same temperature. Completion of reaction was monitored by TLC. After completion reaction mass was quenched with water and partitioned with EtOAc. The organic layer was, dried over sodium sulfate and concentrated under reduced pressure to give crude product that was purified by silica gel(100-200 mesh) chromatography, eluent 3% EtOAc/Hexane to obtain (2-bromoethyl)(4-bromophenyl)sulfane (0.24g, 25%) as colourless liquid.

MS: 295.2[M⁺+1]

### Step-2: Synthesis of 1-(2-bromoethylsulfonyl)-4-bromobenzene

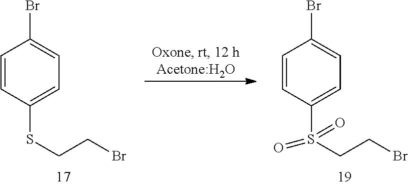 [SMILES: BrCCSc1ccc(Br)cc1 ; O=S(=O)(CCBr)c1ccc(Br)cc1]

To a stirred solution of (2-bromoethyl)(4-bromophenyl)sulfane (0.03 g, 1.01 mmol) in acetone (10 mL) was added Oxone (0.93 g, 3.04 mmol) in water (2mL) then, reaction mass was stirred at room temperature for 12h. Reaction was monitored by TLC. On completion reaction was concentrated under reduced pressure and extracted with ethyl acetate. The organic layer was washed with water, brine, dried over sodium sulphate, concentrated under reduced pressure obtained crude which was purified column chromatography (100-200 Mesh); eluent 20% EtOAc/Hexane to afford 1-(2-bromoethylsulfonyl)-4-bromobenzene (0.21g, 63%) as off white solid.

MS: 327.17[M⁺+1]

### Step-3: Synthesis of 1-(2-methoxyethylsulfonyl)-4-bromobenzene

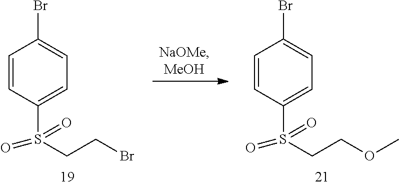

To a stirred solution of 1-(2-bromoethylsulfonyl)-4-bromobenzene (0.1 g, 0.30 mmol) in MeOH (5 mL) was added NaOMe (0.02 g,0.36 mmol) at 0° C. and allowed to stir at RT for 3 h. Completion of reaction was monitored by TLC. Reaction mass was quenched with cold water, product extracted with EtOAc, organic layer was dried over sodium sulphate, concentrated under reduced pressure obtained 1-(2-methoxyethylsulfonyl)-4-bromobenzene (0.05 g, 59%) as off-white solid.

MS: 279.17[M⁺+1]

### Step-4: Synthesis of 1-(4-(2-methoxyethylsulfonyl) phenyl)-3-(6-(4-isopropyl-4H-1,2,4-triazol-3-yl)pyridin-2-yl) imidazolidin-2-one.

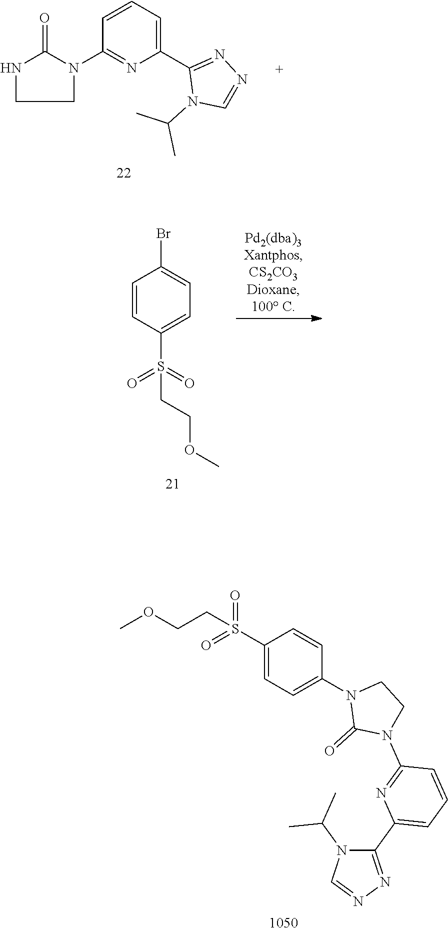 [SMILES: CC(C)n1cnnc1-c1cccc(N2CCNC2=O)n1 ; COCCS(=O)(=O)c1ccc(Br)cc1 ; COCCS(=O)(=O)c1ccc(N2CCN(c3cccc(-c4nncn4C(C)C)n3)C2=O)cc1]

To a stirred solution of 1-(2-methoxyethylsulfonyl)-4-bromobenzene (0.05 g, 0.179 mmol) and 1-(6-(4-isopropyl-4H-1,2,4-triazol-3-yl)pyridin-2-yl)imidazolidin-2-one (0.047 g, 0.202 mmol) in Dioxane (10 mL) was added CS₂CO₃ (0.18 g, 0.551 mmol), reaction mass was degassed with nitrogen for 30 min. Then, Pd₂(dba)₃ (0.017 g, 0.018 mmol) and xantphos (0.021 g, 0.036 mmol) were added to it and stirred at 100° C. for 16h. Reaction was monitored by TLC. On completion reaction was diluted with water , extracted with EtOAc .The organic layer was washed with brine, dried over sodium sulphate, concentrated under reduced pressure obtained crude which was purified column chromatography (100-200 Mesh); eluent 3% MeOH/DCM to afford 1-(4-(2-methoxyethylsulfonyl)phenyl)-3-(6-(4-isopropyl-4H-1,2,4-triazol-3-yl)pyridin-2-yl)imidazolidin-2-one (0.03g, 38%) as off white solid.

MS: 471.17 [M⁺+1].

### Example 1056

### 2-(4-(3-(6-(4-isopropyl-4H-1,2,4-triazol-3-yl)pyridin-2-yl)-2-oxoimidazolidin-1-yl) phenylamino) -2-cyclopropylacetonitrile

### Step 1: 2-(4-bromophenylamino)-2-cyclopropylacetonitrile

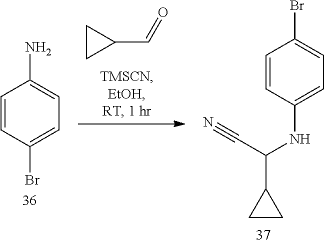 [SMILES: Nc1ccc(Br)cc1 ; O=CC1CC1 ; N#CC(Nc1ccc(Br)cc1)C1CC1]

To a stirred solution of bromo aniline (1.0 g, 5.81 mmol), TMSCN (0.63 g, 6.39 mmol) and cyclopropanecarbaldehyde (0.44 g, 6.39 mmol) were taken into vial containing in EtOH (5 ml) at 0° C. then stir for lhr at RT. Reaction was monitored by TLC. On completion reaction mass was concentrated under reduced pressure. Reaction mixture was quenched with H₂O and extracted with ethyl acetate. The organic layer was washed with water, brine, dried over sodium sulphate, concentrated under reduced pressure obtained crude which was purified by column chromatography; eluent 18% EtOAc/Hexane to afford 2-(4-bromophenylamino)-2-cyclopropylacetonitrile (0.8 g, 54.7%) as off transparent liquid. MS: 251,253 [M⁺+1, M⁺+2]

### Step 2: tert-butyl 4-bromophenylcyano (cyclopropyl) methylcarbamate

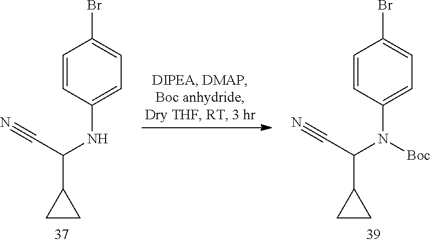 [SMILES: N#CC(Nc1ccc(Br)cc1)C1CC1 ; CC(C)(C)OC(=O)N(c1ccc(Br)cc1)C(C#N)C1CC1]

To a stirred solution of 2-(4-bromophenylamino)-2-cyclopropylacetonitrile (0.5 g, 2.0 mmol), DIPEA (0.515 g, 4.0 mmol) and DMAP (0.05 g, 0.4 mmol) were taken into vial containing in THF (5 ml) at 0° C. than added Boc anhydride and stir for 3hr at RT. Reaction was monitored by TLC. On completion reaction mass was concentrated under reduced pressure. Reaction mixture was quenched with H₂O and extracted with ethyl acetate. The organic layer was washed with water, brine, dried over sodium sulphate, concentrated under reduced pressure obtained crude which was purified by column chromatography; eluent 18% EtOAc/Hexane to afford tert-butyl 4-bromophenylcyano(cyclopropyl)methylcarbamate (0.8 g, 54.7%) as white solid.

MS: 351, 353 [M⁺+1, M⁺+2]

### Step 3: tert-butyl cyano (cyclopropyl) methyl4-(3-(6-(4-isopropyl-4H-1,2,4-triazol-3-yl) pyridin-2-yl)-2-oxoimidazolidin-1-yl) phenylcarbamate

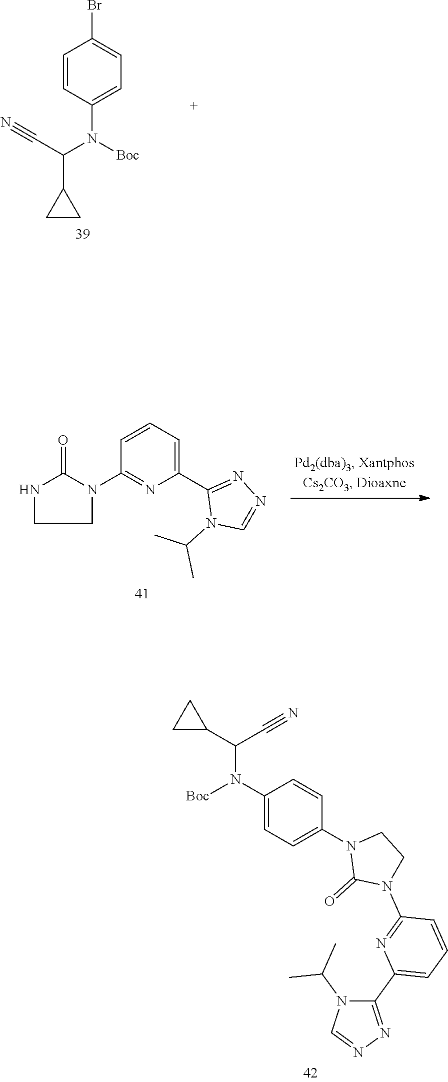 [SMILES: CC(C)(C)OC(=O)N(c1ccc(Br)cc1)C(C#N)C1CC1 ; CC(C)n1cnnc1-c1cccc(N2CCNC2=O)n1 ; CC(C)n1cnnc1-c1cccc(N2CCN(c3ccc(N(C(=O)OC(C)(C)C)C(C#N)C4CC4)cc3)C2=O)n1]

To a stirred solution of tert-butyl 4-bromophenylcyano (cyclopropyl) methylcarbamate (0.05 g, 0.142mmol), 1-(6-(4-isopropyl-4H-1,2,4-triazol-3-yl) pyridin-2-yl) imidazolidin-2-one (0.039 g, 0.142 mmol) and CS₂CO₃ (0.139g, 0.428 mmol) in Dioxane (5 mL) and reaction mass was purged with nitrogen for 30 min. Then, Xantphos (0.016 g, 0.028 mmol) and Pd₂dba₃ (0.013 g, 0.014 mmol) was added to it and stirred at 100° C. for 2 hr. Reaction was monitored by TLC. On completion reaction was quenched with water and extracted with ethyl acetate and dried over sodium sulphate, organic layer evaporate under reduced pressure to obtained crude which was purified by column chromatography (100-200 Mesh); eluent 3.6% MeOH/DCM to afford tert-butyl cyano(cyclopropyl)methyl4-(3-(6-(4-isopropyl-4H-1,2,4-triazol-3 -yl)pyridin-2-yl)-2-oxoimidazolidin- 1-yl)phenylc arbamate (0.010g, 13.51%) as off white solid.

MS: 543.20[M⁺+1]

### Step 4: 2-(4-(3-(6-(4-isopropyl-4H-1,2,4-triazol-3-yl) pyridin-2-yl)-2-oxoimidazolidin-1-yl) phenyl amino)-2-cyclopropylacetonitrile

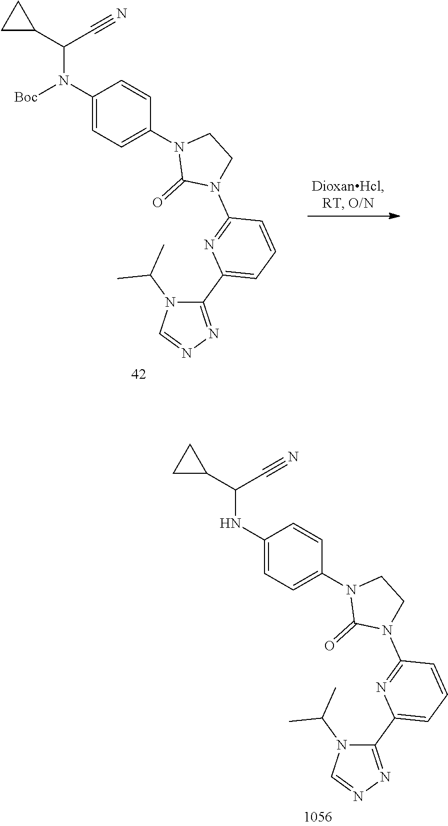 [SMILES: CC(C)n1cnnc1-c1cccc(N2CCN(c3ccc(N(C(=O)OC(C)(C)C)C(C#N)C4CC4)cc3)C2=O)n1 ; CC(C)n1cnnc1-c1cccc(N2CCN(c3ccc(NC(C#N)C4CC4)cc3)C2=O)n1]

To a stirred solution of tert-butyl cyano(cyclopropyl)methyl4-(3-(6-(4-isopropyl-4H-1,2,4-triazol-3-yl)pyridin-2-yl)-2-oxoimidazolidin-1-yl)phenylcarbamate (0.055 g, 1.30 mmol) in Dioxan (0.5 mL) at 0° C. then added dioxane. HCl (2 ml, 13.09 mmol) and stir for overnight at RT. Reaction was monitored by TLC. On completion reaction mass was concentrated under reduced pressure. Reaction mixture was quenched with sodium bicarbonate and extracted with ethyl acetate. The organic layer was washed with water, brine, dried over sodium sulphate, concentrated under reduced pressure to obtained crude which was purified by column chromatography; eluent 4.5% MeOH/DCM to afford 2-(4-(3-(6-(4-isopropyl-4H-1,2,4-triazol-3-yl) pyridin-2-yl)-2-oxoimidazolidin-1-yl) phenylamino)-2-cyclopropylacetonitrile (10.0 mg, 22.72%) as white solid.

MS: 443.20 [M⁺+1]

### Example 1063

### 1-(6-(4-isopropyl-4H-1,2,4-triazol-3-yl)pyridin-2-yl)-3-(4-(methylsulfonyl)-3-(piperidin-1-yl)phenyl)imidazolidin-2-one

### Step 1: Synthesis of 3-fluoro-4-(methylsulfonyl) benzenamine

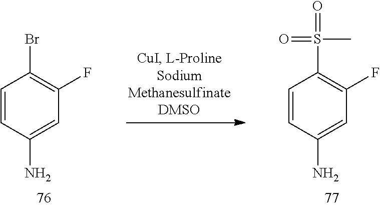

A mixture of 4-bromo-3-fluorobenzenamine (500 mg, 2.6 mmol), sodium methanesulfinate (400 mg, 3.95 mmol), copper iodide (100 mg, 0.8 mmol), L-proline (179 mg, 1.6 mmol), and 4 mL of DMSO in a sealed tube was heated to 80 or 95° C. under argon for 10 h. The cooled mixture was partitioned between ethyl acetate and water. The organic layer was separated, and the aqueous layer was extracted with ethyl acetate twice. The combined organic layers were washed with brine, dried over MgSO_{4,} and concentrated in vacuo. The residual oil was loaded on a silica gel column and eluted with 4:1 petroleum ether/ethyl acetate to afford the product 3-fluoro-4-(methylsulfonyl)benzenamine. Yield: 380 mg, 76%.

MS: 190.03 [M⁺+1].

### Step 2: Synthesis of 4-bromo-2-fluoro-1-(methylsulfonyl) benzene

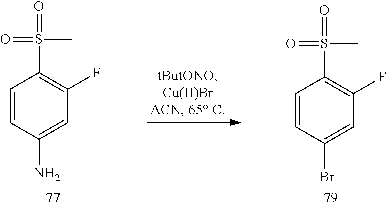

To a solution of copper (II) bromide (0.24 g, 1.07 mmol) in acetonitrile (6 mL) was added tert-butyl nitrite (0.11 g, 1.07 mmol). The mixture was stirred for 5 minutes. Then a solution of 3-fluoro-4-(methylsulfonyl)benzenamine (0.1 g, 0.53 mmol) in acetonitrile (5 mL) was added dropwise. Stirring was continued at room temperature for 2 hours. The reaction is exothermic. After concentrating the solvent, the residue was diluted with ethyl acetate, washed with water, then dried over anhydrous sodium sulphate. The dried solution was concentrated in vacuo, and the residue was purified by chromatography on silica gel with petroleum/ethyl acetate to give 4-bromo-2-fluoro-1-(methylsulfonyl)benzene as white solid.

Yield: 80 mg, 60%.

MS: 252.9 [M⁺+1].

### Step 3: Synthesis of 1-(3-fluoro-4-(methylsulfonyl) phenyl)-3-(6-(4-isopropyl-4H-1,2,4-triazol-3-yl)pyridin-2-yl)imidazolidin-2-one

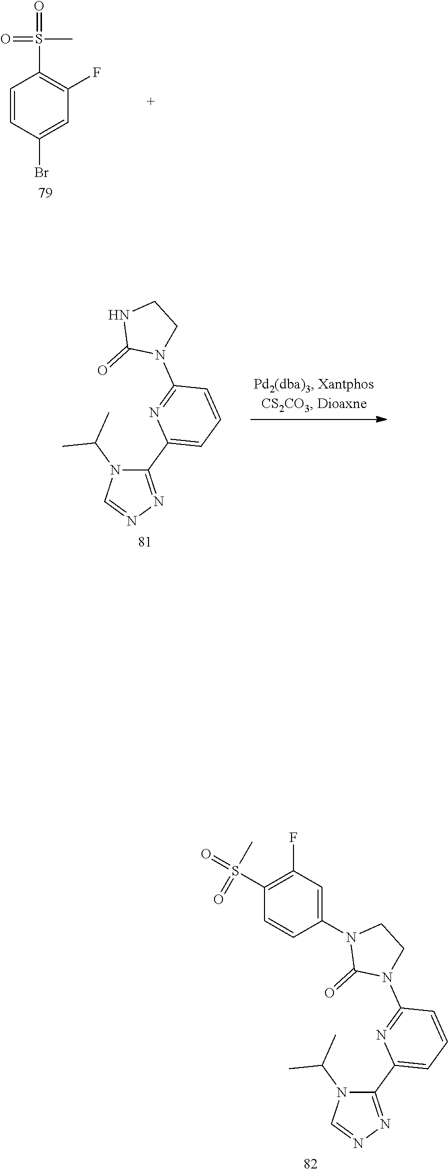 [SMILES: CS(=O)(=O)c1ccc(Br)cc1F ; CC(C)n1cnnc1-c1cccc(N2CCNC2=O)n1 ; CC(C)n1cnnc1-c1cccc(N2CCN(c3ccc(S(C)(=O)=O)c(F)c3)C2=O)n1]

A sealed tube was charged with 4-bromo-2-fluoro-1-(methylsulfonyl)benzene (0.1 g, 0.40 mmol), 1-(6-(4-isopropyl-4H-1,2,4-triazol-3-yl)pyridin-2-yl)imidazolidin-2-one (0.10 g, 0.40 mmol) and dioxane. The solution was rigorously degassed by nitrogen. To this degassed solution tris(dibenzylideneacetone)dipalladium(0) (0.035 g, 0.04 mmol), xantphos (0.034 g, 0.06 mmol), CS₂CO₃ (0.262 g, 0.80 mmol) was added and capped with a teflon screw cap. The mixture was stirred at 100° C. for 4h. On completion, the reaction was diluted with water, extracted with EtOAc. The organic layer was washed with brine, dried over sodium sulphate, concentrated under reduced pressure obtained crude which was purified column chromatography (100-200 Mesh); eluent 4% MeOH/DCM to afford 1-(3-fluoro-4-(methylsulfonyl)phenyl)-3 -(6-(4 -is opropyl-4H-1,2,4 -triazol-3 -yl)pyridin-2-yl)imidazolidin-2-one (0.101 g, 58%) as off white solid.

MS: 445.14 [M⁺+1].

### Step 4: Synthesis of 1-(6-(4-isopropyl-4H-1,2,4-triazol-3-yl)pyridin-2-yl)-3-(4-(methylsulfonyl)-3-(piperidin-1-yl)phenyl)imidazolidin-2-one

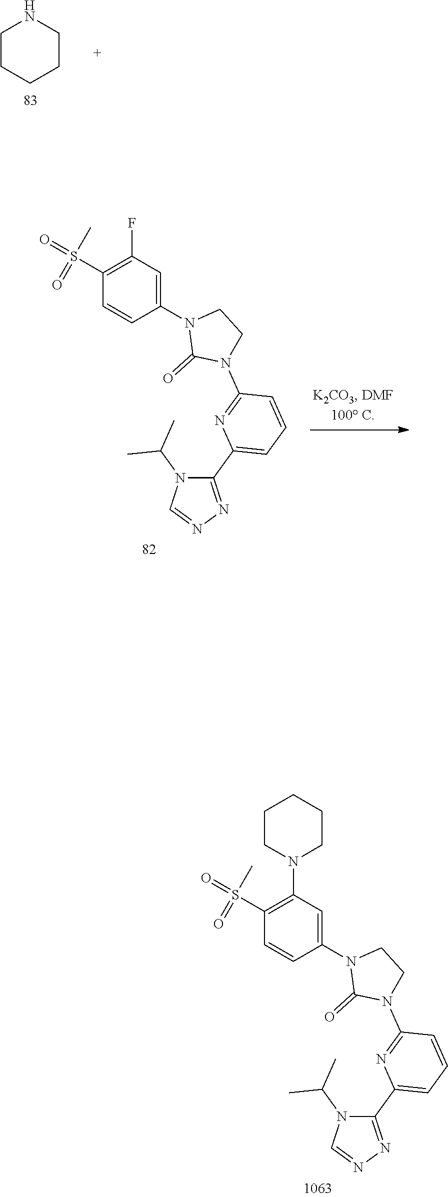 [SMILES: C1CCNCC1 ; CC(C)n1cnnc1-c1cccc(N2CCN(c3ccc(S(C)(=O)=O)c(F)c3)C2=O)n1 ; CC(C)n1cnnc1-c1cccc(N2CCN(c3ccc(S(C)(=O)=O)c(N4CCCCC4)c3)C2=O)n1]

To a solution of 1-(3-fluoro-4-(methylsulfonyl)phenyl)-3-(6-(4-isopropyl-4H-1,2,4-triazol-3-yl)pyridin-2-yl)imidazolidin-2-one (50 mg, 0.11 mmol) in DMF piperidine (0.034 g, 0.45 mmol) was added followed by potassium carbonate (0.030 mg, 0.22 mmol). The resulting mixture was heated at 100° c for 4 h. The mixture was stirred at 100° C. for 4h. On completion, the reaction was diluted with water, extracted with EtOAc. The organic layer was washed with brine, dried over sodium sulphate, concentrated under reduced pressure obtained crude which was purified column chromatography (100-200 Mesh); eluent 4% MeOH/DCM to afford 1-(6-(4-isopropyl-4H-1,2,4-triazol-3-yl)pyridin-2-yl)-3-(4-(methylsulfonyl)-3-(piperidin-1-yl)phenyl)imidazolidin-2-one (0.015 g, 27% yield) as off white solid.

MS: 496.21 [M⁺+1].

### Example 1071

### 1-(4-(3-(6-(4-isopropyl-4H-1,2,4-triazol-3-yl)pyridin-2-yl)-2-oxoimidazolidin-1-yl)phenyl)azetidine-3-carbonitrile

### Step 1: 1-(4-bromophenyl)azetidine-3-carbonitrile

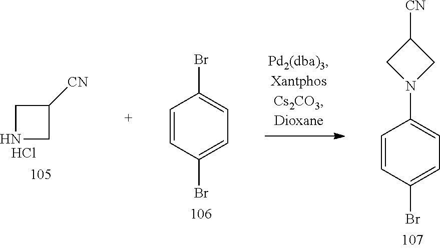 [SMILES: N#CC1CNC1 ; Cl ; Brc1ccc(Br)cc1 ; N#CC1CN(c2ccc(Br)cc2)C1]

To a stirred solution of azetidine-3-carbonitrile hydrochloride (0.1 g, 0.843 mmol) and 1,4-dibromobenzene (0.2 g, 0.843 mmol) in Dioxane (5 mL) was added CS₂CO₃ (0.68 g, 2.109 mmol), reaction mass was degassed with nitrogen for 30 min. Then, Pd₂(dba)₃ (0.077 g, 0.084 mmol) and Xantphos (0.073 g, 0.126 mmol) were added to it and stirred at 100° C. for 16h. Reaction was monitored by TLC. On completion reaction was diluted with water, extracted with EtOAc .The organic layer was washed with brine, dried over sodium sulphate, concentrated under reduced pressure obtained crude which was purified column chromatography (100-200 Mesh); eluent 30% Ethyl acetate/ Hexane to afford 1-(4-bromophenyl)azetidine-3-carbonitrile (0.1 g, 50%) as off white solid.

MS: 236.9 & 238.9[M⁺+1 & M⁺+3]

### Step 2: 1-(4-(3-(6-(4-isopropyl-4H-1,2,4-triazol-3-yl)pyridin-2-yl)-2-oxoimidazolidin-1-yl)phenyl)azetidine-3-carbonitrile

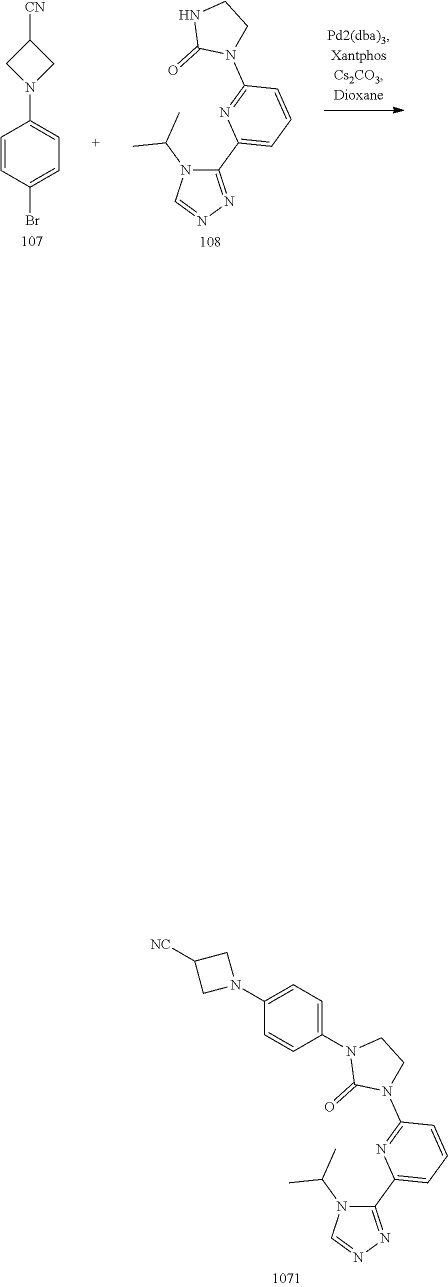 [SMILES: N#CC1CN(c2ccc(Br)cc2)C1 ; CC(C)n1cnnc1-c1cccc(N2CCNC2=O)n1 ; CC(C)n1cnnc1-c1cccc(N2CCN(c3ccc(N4CC(C#N)C4)cc3)C2=O)n1]

To a stirred solution of 1-(4-bromophenyl)azetidine-3-carbonitrile (0.1 g, 0.422 mmol) and 1-(6-(4-isopropyl-4H-1,2,4-triazol-3-yl)pyridin-2-yl)imidazolidin-2-one (0.11 g, 0.422 mmol) in dioxane (5 mL) was added CS₂CO₃ (0.34 g, 1.05 mmol), reaction mass was degassed with nitrogen for 30 min. Then, Pd₂(dba)₃ (0.038 g, 0.042mmol) and Xantphos (0.036 g, 0.633 mmol) were added to it and stirred at 100° C. for 16h. Reaction was monitored by TLC. On completion reaction was diluted with water , extracted with EtOAc .The organic layer was washed with brine, dried over sodium sulphate, concentrated under reduced pressure obtained crude which was purified column chromatography (100-200 Mesh); eluent 30% Ethyl acetate/ Hexane to afford 1-(4-bromophenyl)azetidine-3-carbonitrile (0.036 g, 25%) as off white solid.

MS: 429.5[M⁺+1]

By following above synthetic procedure examples 1074,1088,1089,1090,1091,1093,1094,1096,1115,1122,1107 and 1027 were synthesized by changing the substituents at step 2.

### Example1082

### 1-(4-(3-(6-(4-isopropyl-4H-1,2,4-triazol-3-yl)pyridin-2-yl)-2-oxoimidazolidin-1-yl)phenyl)-N-methylpiperidine-4-carboxamide

### Step 1: Synthesis of 1-(4-bromophenyl)-N-methylpiperidine-4-carboxamide

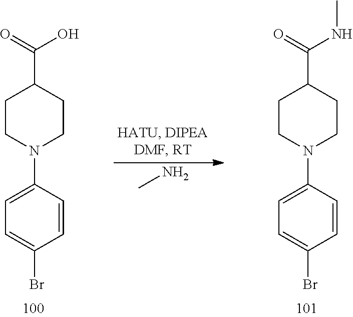

To a stirred solution of 1-(4-bromophenyl)piperidine-4-carboxylic acid (0.5 g, 1.76 mmol), DIPEA (0.68 g, 5.30 mmol) and HATU (0.80 g, 2.12 mmol) were taken into vial containing in DMF (5 ml) at 0° C. then added methyl amine (0.060 g, 1.95mmoL) and stir for 3hr at RT. Reaction was monitored by TLC. On completion reaction mass was quenched with H₂O and extracted with ethyl acetate. The organic layer was washed with water, brine, dried over sodium sulphate, concentrated under reduced pressure obtained crude which was purified by column chromatography; eluent 4% MeOH/DCM to afford 1-(4-bromophenyl)-N-methylpiperidine-4-carboxamide (0.3 g, 57.0%) as Off-white solid.

MS: 297,299 [M⁺+1, M⁺+2]

### Step 2: Synthesis of 1-(4-(3-(6-(4-isopropyl-4H-1,2,4-triazol-3-yl)pyridin-2-yl)-2-oxoimidazolidin-1-yl)phenyl)-N-methylpiperidine-4-carboxamide

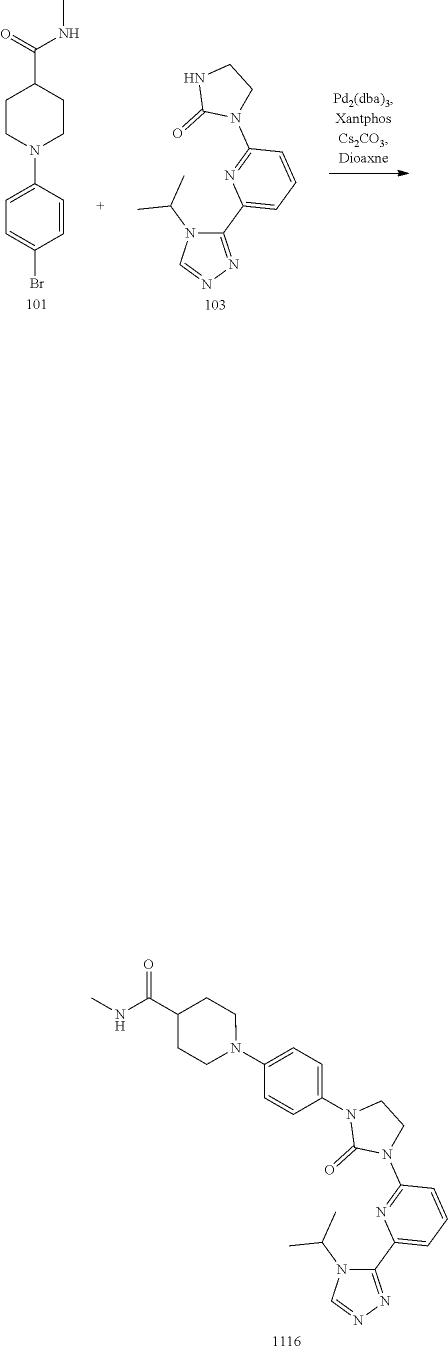 [SMILES: CNC(=O)C1CCN(c2ccc(Br)cc2)CC1 ; CC(C)n1cnnc1-c1cccc(N2CCNC2=O)n1 ; CNC(=O)C1CCN(c2ccc(N3CCN(c4cccc(-c5nncn5C(C)C)n4)C3=O)cc2)CC1]

A sealed tube was charged with 1-(4-bromophenyl)-N-methylpiperidine-4-carboxamide (0.1 g, 0.33 mmol), 1-(6-(4-isopropyl-4H-1,2,4-triazol-3 -yl)pyridin-2-yl)imidazolidin-2-one (0.091 g, 0.33 mmol) and dioxane . The solution was rigorously degassed by nitrogen. To this degassed solution tris(dibenzylideneacetone)dipalladium(0) (0.030 g, 0.033 mmol), xantphos (0.039 g, 0.067 mmol), CS₂CO₃ (0.33 g, 1.013 mmol) was added and capped with a teflon screw cap. The mixture was stirred at 100° C. for 4h. On completion, the reaction was diluted with water, extracted with EtOAc. The organic layer was washed with brine, dried over sodium sulphate, concentrated under reduced pressure obtained crude which was purified column chromatography (100-200 Mesh); eluent 5% MeOH/DCM to afford 1-(4-(3-(6-(4-isopropyl-4H- 1,2,4-triazol-3 -yl)pyridin-2-yl)-2-oxoimidazolidin-1-yl)phenyl)-N-methylpiperidine-4-carboxamide (0.065 g, 39%) as off white solid.

MS: 489.26 [M⁺+1].

### Example1097

### 1-(4-(3-(6-(4-isopropyl-4H-1,2,4-triazol-3-yl)pyridin-2-yl)-2-oxoimidazolidin-1-yl)phenyl)-N,N-dimethylpiperidine-4-carboxamide

### Step 1: Synthesis of 1-(4-bromophenyl) piperidine-4-carboxylic acid

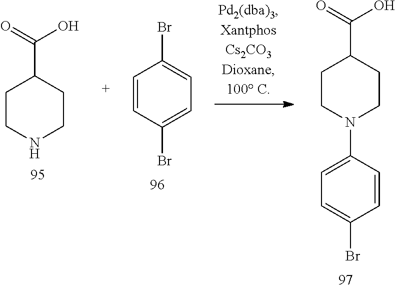 [SMILES: O=C(O)C1CCNCC1 ; Brc1ccc(Br)cc1 ; O=C(O)C1CCN(c2ccc(Br)cc2)CC1]

A sealed tube was charged with piperidine-4-carboxylic acid (1.0 g, 7.75 mmol), 1,4-dibromobenzene (1.99 g, 8.52 mmol) and dioxane. The solution was vigorously degassed by nitrogen. To this degassed solution tris(dibenzylideneacetone)dipalladium(0) (0.709 g, 0.775 mmol), xantphos (0.897 g, 1.155 mmol), CS₂CO₃ (7.58 g, 23.25 mmol) was added and capped with a teflon screw cap. The mixture was stirred at 100° C. for 4h. On completion, the reaction was diluted with water, extracted with EtOAc. The organic layer was washed with brine, dried over sodium sulphate, concentrated under reduced pressure obtained crude which was purified column chromatography (100-200 Mesh); eluent 3% MeOH/DCM to afford 1-(4-bromophenyl)piperidine-4-carboxylic acid (1.2 g, 55%) as off white solid.

MS: 284 [M⁺+1].

### Step 1: Synthesis of 1-(4-bromophenyl)-N,N-dimethylpiperidine-4-carboxamide

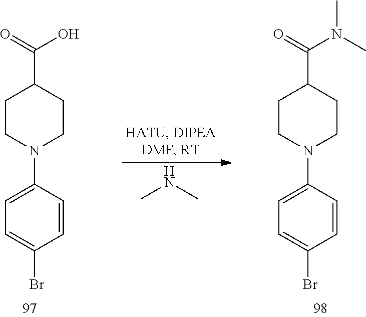

To a stirred solution of 1-(4-bromophenyl)piperidine-4-carboxylic acid (0.5 g, 1.76 mmol), DIPEA (0.68 g, 5.30 mmol) and HATU (0.80 g, 2.12 mmol) were taken into vial containing in DMF (5 ml) at 0° C. then added dimethyl amine (0.087 g, 1.94mmoL) and stir for 3hr at RT. Reaction was monitored by TLC. On completion reaction mass was quenched with H₂O and extracted with ethyl acetate. The organic layer was washed with water, brine, dried over sodium sulphate, concentrated under reduced pressure obtained crude which was purified by column chromatography; eluent 4% MeOH/DCM to afford 1-(4-bromophenyl)-N,N-dimethylpiperidine-4-carboxamide (0.4 g, 73.0%) as Off-white solid.

MS: 311,313 [M⁺+1, M⁺+2]

### Step 3: Synthesis of 1-(4-(3-(6-(4-isopropyl-4H-1,2,4-triazol-3-yl)pyridin-2-yl)-2-oxoimidazolidin-1-yl)phenyl)-N,N-dimethylpiperidine-4-carboxamide

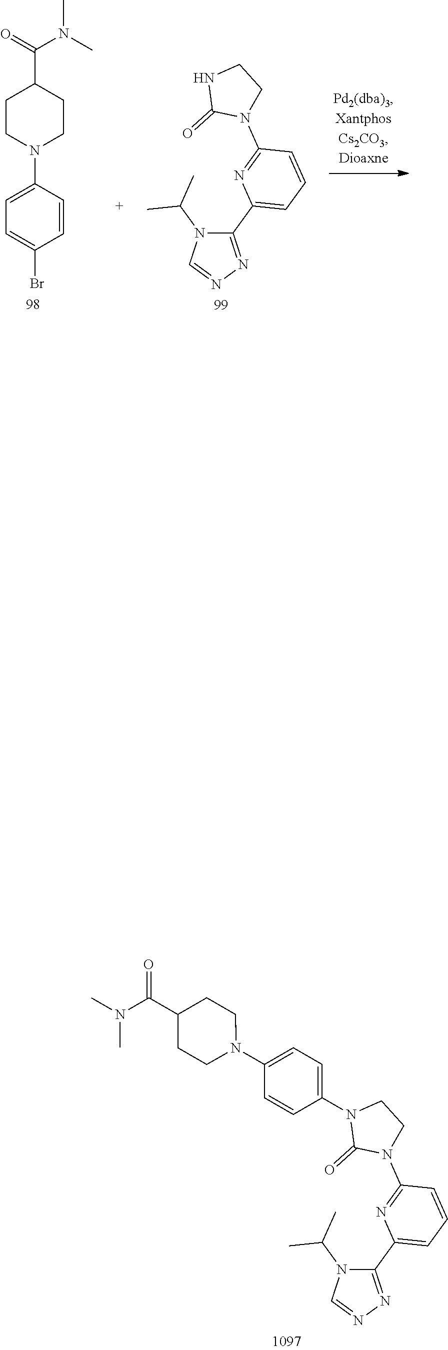 [SMILES: CN(C)C(=O)C1CCN(c2ccc(Br)cc2)CC1 ; CC(C)n1cnnc1-c1cccc(N2CCNC2=O)n1 ; CC(C)n1cnnc1-c1cccc(N2CCN(c3ccc(N4CCC(C(=O)N(C)C)CC4)cc3)C2=O)n1]

A sealed tube was charged with 1-(4-bromophenyl)-N,N-dimethylpiperidine-4-carboxamide (0.1 g, 0.32 mmol), 1-(6-(4-isopropyl-4H-1,2,4-triazol-3-yl)pyridin-2-yl)imidazolidin-2-one (0.087 g, 0.40 mmol) and dioxane. The solution was rigorously degassed by nitrogen. To this degassed solution tris(dibenzylideneacetone)dipalladium(0) (0.029 g, 0.032 mmol), xantphos (0.037 g, 0.064 mmol), CS₂CO₃ (0.315 g, 0.96 mmol) was added and capped with a teflon screw cap. The mixture was stirred at 100° C. for 4h. On completion, the reaction was diluted with water, extracted with EtOAc. The organic layer was washed with brine, dried over sodium sulphate, concentrated under reduced pressure obtained crude which was purified column chromatography (100-200 Mesh); eluent 5% MeOH/DCM to afford 1-(4-(3-(6-(4-isopropyl-4H- 1,2,4-triazol-3 -yl)pyridin-2-yl)-2-oxoimidazolidin-1-yl)phenyl)-N,N-dimethylpiperidine-4-carboxamide (0.06 g, 37%) as off white solid.

MS: 503.28 [M⁺+1].

By following above synthetic procedure examples 1082, 1106,1030,103,1139, and 1140 were also synthesized by changing the substituents.

### Examples 1

### ASK1 Biochemical Assay

ASK1 biochemical assay was standardized using recombinant ASK1 (Carna Biosciences), substrate and detection reagents from Cisbio HRTF KinEASE-STK S3 kit. Enzyme concentration, ATP, biotinylated STK substrate and STK antibodies were titrated for enzyme assay. Assay was carried out in ASK1 kinase buffer, 10 mM MOPS pH-7, 10 mM Mg-acetate, 1 mM DTT, 0.025% NP40, 1.5% glycerol and 0.05% BSA. DMSO was used as vehicle control with a final concentration of 1%. A 5 ng of ASK1 enzyme was incubated with compound for 1 hour at 23° C. on a shaker at 100rpm. ATP and biotin substrate was added at a final concentration of 100 μM and 1 μM and incubated for 3 hours at 23° C. on a shaker at 100rpm. Detection reagents SA-XL665 (0.0625 μM) and Cryptate antibody were added and incubated for 1 hour at room temperature on a shaker at 100rpm. Delta F ratios were calculated from data obtained at excitation (337 nm) and emission wavelengths (665 nm and 620-10 nm). Substrate blank values was considered as negative control. Values from negative control was substrated from vehicle control and compound treated wells. Enzyme inhibition by compounds were calculated from vehicle controls, which represent uninterrupted enzyme's activity.

### Examples 2

### ASK1 Cell Based Assay

HEK₂₉₃T-AP1 cells were seeded at a density of 20,000 cells per well in a poly-d-lysine coated 96 well microplate. Next day, cells were transfected with hASK1-pcDNA3.1 vector using lipofectamine 3000. After 24 hours of transfection, cells were treated with compounds for 24 hours in assay medium. DMSO was used as a vehicle control with a final DMSO concentration of 0.2%. On 4th day, medium was discarded and cells were activated with 1 mM hydrogen peroxide for 1 hour in the presence of compounds. Cells were lysed for 1 hour with 1× lysis buffer on a shaker at 100 rpm. Sixteen microliters of lysate was collected into a 384 well small volume plate. Phospho p38 levels in the lysate was detected by using CisbioPhospho-p38 MAPK (Thr180/Tyr182) #64P38PEG.Four microliters of phospho p38 antibodies labelled with europium cryptate and d2 acceptor, were added to the lysate and incubated overnight. TR-FRET was measured at an excitation of 335 nm and emission was measured at 615 nm and 665 nm in BMG FLUOstar multimode reader.

Compounds were assessed for ASK1 inhibition based on phospho p38 read out. Human ASK1 transfected cells exposed to hydrogen peroxide exhibit uninhibited phospho p38 levels was considered as vehicle control and hASK1 transfected cells without addition of hydrogen peroxide was considered as background controls. Background control values were susbtracted from vehicle control as well as from compound treated wells. Percentage inhibition of ASK1 by compounds was calculated from vehicle controls, which were considered for 100% ASK1 activity.

| | Biochemical | Cell Based |
|---|---|---|
| | Assay | Assay |
| S.No. | IC50 (μM) | IC50 (μM) |
| 1001. | 1.3 | |
| 1002. | 0.07 | >1 |
| 1003. | >1 | |
| 1004. | >1 | |
| 1005. | <1 | |
| 1006. | <1 | |
| 1007. | 0.03 | >1 |
| 1008. | >1 | |
| 1009. | 0.095 | |
| 1010. | 0.025 | >1 |
| 1011. | <1 | >10 |
| 1012. | <1 | |
| 1013. | <0.1 | <1 |
| 1014. | >0.1 | >10 |
| 1015. | >1 | |
| 1016. | >1 | |
| 1017. | >1 | |
| 1018. | <0.1 | >1 |
| 1019. | >0.1 | >10 |
| 1020. | <0.1 | >10 |
| 1021. | >1 | >10 |
| 1022. | <0.1 | >1 |
| 1023. | <0.1 | >1 |
| 1024. | 0.1 | |
| 1025. | <0.1 | <1 |
| 1026. | >0.1 | |
| 1027. | <0.1 | >1 |
| 1028. | >1 | |
| 1029. | <0.1 | >1 |
| 1030. | >1 | |
| 1031. | >1 | |
| 1032. | <0.1 | >1 |
| 1033. | >0.1 | |
| 1034. | <0.1 | >1 |
| 1035. | <0.1 | >10 |
| 1036. | <0.1 | <1 |
| 1037. | <0.1 | <1 |
| 1038. | <0.1 | <1 |
| 1039. | <0.1 | >1 |
| 1040. | <0.1 | >10 |
| 1041. | <0.1 | >1 |
| 1042. | >0.1 | |
| 1043. | <0.1 | 0.14 |
| 1044. | <0.1 | >1 |
| 1045. | <0.1 | <1 |
| 1046. | >0.1 | |
| 1047. | <0.1 | >1 |
| 1048. | <0.1 | >1 |
| 1049. | <0.1 | |
| 1050. | <0.1 | >1 |
| 1051. | <0.1 | <1 |
| 1052. | <0.1 | >1 |
| 1053. | <0.1 | >1 |
| 1054. | >0.1 | |
| 1055. | <0.1 | >1 |
| 1056. | >0.1 | >1 |
| 1057. | <0.1 | >1 |
| 1058. | >0.1 | |
| 1059. | <0.1 | >1 |
| 1060. | <0.1 | |
| 1061. | <0.1 | >1 |
| 1062. | >1 | |
| 1063. | <0.1 | >1 |
| 1064. | >0.1 | |
| 1065. | >1 | |
| 1066. | <0.1 | >1 |
| 1067. | <0.1 | >0.1 |
| 1068. | <0.1 | >0.1 |
| 1069. | <0.1 | >0.1 |
| 1070. | <0.1 | |
| 1071. | <0.1 | <0.1 |
| 1072. | >0.1 | |
| 1073. | <0.1 | >0.1 |
| 1074. | <0.1 | >0.1 |
| 1075. | <0.1 | >1 |
| 1076. | <0.1 | >1 |
| 1077. | <0.1 | >0.1 |
| 1078. | <0.1 | >0.1 |
| 1079. | <0.1 | |
| 1080. | <0.1 | >1 |
| 1081. | <0.05 | >0.1 |
| 1082. | <0.05 | <0.1 |
| 1083. | <0.1 | >1 |
| 1084. | <0.1 | >1 |
| 1085. | <0.1 | >0.1 |
| 1086. | <0.1 | 1 |
| 1087. | <0.1 | >0.1 |
| 1088. | <0.1 | >1 |
| 1089. | <0.1 | 1 |
| 1090. | <0.1 | >1 |
| 1091. | <0.1 | >1 |
| 1092. | <0.1 | >1 |
| 1093. | <0.1 | >0.1 |
| 1094. | <0.1 | >1 |
| 1095. | <0.1 | >1 |
| 1096. | <0.1 | >0.1 |
| 1097. | <0.05 | <0.1 |
| 1098. | <0.1 | >1 |
| 1099. | <0.1 | |
| 1100. | <0.1 | |
| 1101. | <0.05 | 0.19 |
| 1102. | <0.1 | |
| 1103. | <0.1 | >0.1 |
| 1104. | <0.1 | >0.1 |
| 1105. | <0.1 | >0.1 |
| 1106. | <0.1 | >1 |
| 1107. | <0.05 | <0.1 |
| 1108. | >0.1 | >1 |
| 1109. | <0.1 | >1 |
| 1110. | <0.1 | >1 |
| 1111. | <0.1 | |
| 1112. | <0.1 | >1 |
| 1113. | <0.1 | >1 |
| 1114. | <0.01 | >1 |
| 1115. | <0.1 | >1 |
| 1116. | <0.05 | <0.1 |
| 1117. | <0.1 | |
| 1118. | <0.1 | >1 |
| 1119. | <0.05 | 1 |
| 1120. | <0.1 | |
| 1121. | <0.1 | |
| 1122. | <0.1 | 1 |
| 1123. | >0.1 | >1 |
| 1124. | <0.1 | >1 |
| 1125. | <0.1 | >1 |
| 1126. | <0.1 | >1 |
| 1127. | <0.1 | 1 |
| 1128. | <0.1 | >1 |
| 1129. | <0.1 | >1 |
| 1130. | <0.05 | <0.1 |
| 1131. | <0.1 | 1.56 |
| 1132. | >1 | |
| 1133. | <0.1 | >1 |
| 1134. | <0.1 | >1 |
| 1135. | <0.1 | >1 |
| 1136. | <0.1 | >0.1 |
| 1137. | <0.1 | >0.1 |
| 1138. | <0.1 | >0.1 |
| 1139. | <0.1 | >0.1 |
| 1140. | <0.05 | <0.1 |

Compounds were tested for ASK1 inhibition and compared with reference standard, Selonsertib. The IC50s of compounds were tabulated below along with reference standard values generated in the in-house biochemical and cell based screening procedures.

| | ASK1 | ASK1 ph. |
|---|---|---|
| | biochemical | P38 Cell |
| | Assay | based Assay |
| Examples | (IC50, nM) | (IC50, nM) |
| 1043 | 4.1 | 140 |
| 1071 | 3.8 | 69 |
| 1082 | 1.0 | 15 |
| 1097 | 3.8 | 102 |
| Selonsertib | 4.9 | 170 |

Examples mentioned in the above table showed better potency than Selonsertib. They showed 1.2 to 2.7 fold higher potency than Selonsertib in biochemical assay and approximately 1.2 to 11 fold higher potency in phosphor p38 cell based assays.

### Examples 3

### Selectivity Assay

ASK₂ assay was performed to evaluate above mentioned example for its selectivity towards ASK1 with respect ASK 2.

ASK₂ biochemical assay was standardized using ASK₂ (Carna Biosciences), MBP biotin conjugate (Merk life science), ATP, streptavidin surelight APC (PerkinElmer) and Lance Eu-W1024-antiphospho threonine (PerkinElmer). Enzyme concentration, ATP, MBP biotin conjugate and antibodies were titrated for enzyme assay. Assay was carried out in freshly prepared ASK₂ kinase buffer, 5 mM MOPS pH-7.2, 2.5 mM β-glycerophosphate, 5 mM magnesium chloride (MgCl₂), 1 mM EGTA, 0.4 mM EDTA, 0.05 mM DTT and 0.05% BSA. DMSO was used as vehicle control with a final concentration of 1%. A 40 ng of ASK₂ enzyme was incubated with compound for 1 hour at 23° C. on a shaker at 100rpm. ATP and MBP biotin conjugate (as substrate) was added at a final concentration of 100 μM and 0.1mg/ml respectively and incubated for 3 hours at 23° C. on a shaker at 100rpm. Detection reagents streptavidin surelight APC (55.56nM), lance Eu-W1024-antiphospho threonine antibody (1.11nM) and 1X lance detection buffer were added and incubated for 2 hour at room temperature on a shaker at 100rpm. Delta F ratios were calculated from data obtained at excitation (337 nm) and emission wavelengths (665 nm and 620-10 nm). Substrate blank values were considered as negative control. Values from negative control were subtracted from vehicle control and compound treated wells. Enzyme inhibition by compounds was calculated from vehicle controls, which represent uninterrupted enzyme's activity.

| | ASK1 | ASK2 |
|---|---|---|
| | Biochemical | Biochemical |
| | Assay | Assay |
| Examples | IC50 (nM) | IC50 (μM) |
| 1043 | 4.1 | >10 |
| 1071 | 3.8 | >10 |
| 1082 | 1.0 | 8.5 |
| 1097 | 3.8 | 7.9 |

From the above table, it is evident that the exemplary compounds of the present invention show selectivity towards ASK1 with respect to ASK_{2.}

### Examples 4

### Mouse model of Acetaminophen (APAP) induced Hepatotoxicity

Acetaminophen (N-acetyl-p-aminophenol [APAP]) is the most widely used antipyretic and analgesic drug, is considered to be safe at recommended therapeutic concentrations. However, APAP overdose is the most common cause of liver failure. Because APAP hepatotoxicity is dose-dependent and reproducible in animal models, the underlying mechanism has been well characterized. At high doses, cytochrome P-450 enzymes convert APAP into a reactive quinone form, N-acetyl-p-benzoquinone imine (NAPQI). Although N-acetyl-pbenzoquinone imine is inactivated by conjugation with glutathione (GSH), once the pool of GSH is exhausted, any remaining N-acetyl-p-benzoquinone imine covalently binds cellular proteins, resulting in oxidative stress, mitochondrial dysfunction, and DNA damage.

These initial events ultimately may result in direct damage to hepatocytes and cell death. After acute toxic liver injury, several cytokines produced by these injured hepatocytes and nonparenchymal inflammatory cells contribute to APAP-induced hepatotoxicity.

ASK1 is a ubiquitously expressed MAPKKK that is activated by various types of stress, including reactive oxygen species, tumor necrosis factor-_, lipopolysaccharide, endoplasmic reticulum stress, and calcium influx. ASK1 activates the JNK and p38 signaling pathways and is required for both oxidative stress— and cytokine-induced apoptosis. ASK1 is particularly important for the oxidative stress—mediated activation of JNK and p38. Therefore, ASK1 inhibitors are evaluated in APAP induced hepatotoxicity in mouse, events in which ultimately leads to oxidative stress, mitochondrial dysfunction.

Male C57BL/6 mice, 20 to 24 grams body weight at the time of study initiation, were acclimatized at least for 1-3 days prior to experiment initiation. Based on body weight, animal were randomized to different groups and kept for overnight fasting. Animals were treated with test compounds and vehicle for control group. After 30 minutes, APAP (300 mg/kg) was administered by intraperitoneal route (i.p). A vehicle control group was also included in the study as a vehicle control for test compound as well as APAP. All animals were bled 6h post APAP administration from the retro-orbital plexus into appropriately labeled tubes. Serum ALT levels were measured in all groups.

Example 1043, 1071, 1082 and 1097 have showed significant reduction in serum ALT levels compared to vehicle at l0mpk and 30mpk respectively. The reduction is ALT levels were found to be dose dependent and were comparable with reference standard, selonsertib. Data suggests that examples synthesized for apoptosis signal-regulating kinase-1 inhibition have the potential for further development for the treatment of NASH and other related therapeutic indications.

1. 1-(6-(4-isopropyl-4H-1,2,4-triazol-3-yl)pyridin-2-yl)imidazolidin-4-one as inhibitors of apoptosis signal-regulating kinase 1 (“ASK1”), their pharmaceutically acceptable salts and isomers of formula I: 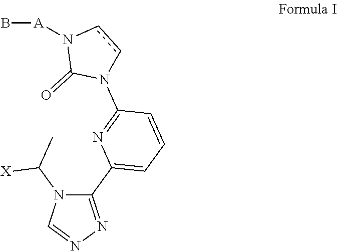
Wherein;
indicates an optional bond
A is absent, H, C1-C₆ alkyl, 5 membered or a 6 membered saturated or unsaturated carbocycle or heterocycle that may be aromatic or aliphatic, which may contain single ring or fused ring and the ring may comprise 1 to 3 heteroatom selected from the group comprising O, N, S, which may be further substituted with R₁, R₁R₂, OH, CH₃, F or Cl; —OR₁, _CO, —NR₁R₂, —SO₂(CH₂)n, —CHOHR₁, —CONR₁R₂, —COR₁, —CN, —CF₃; SO₂NR₁R₂, SO₂R₁, O(CH₂)nSO₂CH₃, NH(CR₁R₂)n, NH(CH₂)nR₁, CH₂SO₂R₁, a 3 to 6 membered carbocycle, a 5 or 6 membered heterocycle containing 1 or 2 heteroatoms selected from O, N or S; the heteroatom may be oxidised;
B is absent, H, SO₂, SO₂(CH₂)n; SO₂R₁, SO₂(CH₂)nOR₁, SO₂NHR₁, NH₂(CH₂)n, CO NH₂(CH₂)n, NH₂(CH₂)n, NH₂(CH₂)nR₁CONH₂, NH₂(CH₂)nCN, NHCHR₁CN, NHR₁, SO₂Me, 3 to 8 carbocyclic ring optionally substituted by R1 or R2 or R3, 3 to 8 membered heterocyclic ring containing 1 to 3 heteroatoms selected from the group comprising O, N, S which may be optionally substituted by R1 or R2 or R3;
Wherein (CH₂)n can form a cyclic structure depending upon the number of carbon atoms;
Wherein A may be connected to the ring though R1 and B may be connected to A through R1;
X is H, CH₃, (CH₂)n, C₁-C₆ straight or branched or cyclic alkyl, CN, (CH₂)CN, OH, (CH₂)nOH, CF3;
R1, R2 and R3 are independently selected from the group comprising H, halo, CN, CF₃, CH₃, hydroxyl, C₁-C₆ alkyl, C₁ to C₆ alkoxy , C₁-C₆alkoxyalkyl and C₁-C₆cyanoalkyl, amino, SO₂, SO₂C₁-C₆ Alkyl, CH₂CF₃, —(CH₂)nCN, —CH₂OMe, —CH₂SO₂Me, NR₄R₅, CONR₄R₅, C₁-C₆ straight or branched or cyclic alkyl, C₂-C₆ straight or branched alkenyl, C₂-C₆ straight or branched alkynyl, halo-C₁-C₆ alkyl, C₁-C₆ alkyloxy; C₁-C₆ alkylamino, heteroalkyl, C₃-C₈ cycloalkyl, C₃-C₈cycloalkenyl, C₃-C₈heterocycloalkyl, C₃-C₈heterocycloalkenyl, carbocycyl, aryl, and heteroaryl; oxidised heteroaryl, oxidise heterocyclyl, heteroaryl or heterocyclyl that may be optionally substituted with R4 and R5;
R4 and R5 may be selected from H, C₁-C₆ alkyl and C₃-C₈ cycloalkyl, NH₂, NH, C₁-C₆ alkyl
n is 0 to 3.

2. The compounds of formula I, as claimed in claim 1, selected from the group comprising:
1001. 1-(6-(1H-imidazol-1-yl)pyrimidin-4-yl)-3-(6-(4-isopropyl-4H-1,2,4-triazol-3-yl)pyridin-2-yl)imidazolidin-2-one;
1002. 1-(3-(1H-imidazol-1-yl)phenyl)-3-(6-(4-isopropyl-4H-1,2,4-triazol-3-yl)pyridin-2-yl)imidazolidin-2-one;
1003. 1-(4-(1H-imidazol-1-yl)pyridin-2-yl)-3-(6-(4-isopropyl-4H-1,2,4-triazol-3-yl)pyridin-2-yl)imidazolidin-2-one;
1004. 1-(6-(1H-imidazol-1-yl)pyrimidin-4-yl)-3-(6-(4-isopropyl-4H-1,2,4-triazol-3-yl)pyridin-2-yl)-1H-imidazol-2(3H)-one;
1005. 1-(3-(4-cyclopropyl-1H-imidazol-1-yl)phenyl)-3-(6-(4-isopropyl-4H-1,2,4-triazol-3-yl)pyridin-2-yl)imidazolidin-2-one;
1006. 1-(3-(1H-imidazol-1-yl)phenyl)-3-(6-(4-isopropyl-4H-1,2,4-triazol-3-yl)pyridin-2-yl)-1H-imidazol-2(3H)-one;
1007. 1-(6-(4-isopropyl-4H-1,2,4-triazol-3-yl)pyridin-2-yl)-3-(4′-(methylsulfonyl)-[1,1′-biphenyl]-3-yl)imidazolidin-2-one;
1008. 1-(2-fluoro-3-(1H-imidazol-1-yl)phenyl)-3-(6-(4-isopropyl-4H-1,2,4-triazol-3-yl)pyridin-2-yl)imidazolidin-2-one;
1009. 1-(6-(4-isopropyl-4H-1,2,4-triazol-3-yl)pyridin-2-yl)-3-(3-(methylsulfonyl)phenyl)imidazolidin-2-one;
1010. 1-(6-(4-isopropyl-4H-1,2,4-triazol-3-yl)pyridin-2-yl)-3-(3-(6-(methylsulfonyl)pyridin-3-yl)phenyl)imidazolidin-2-one;
1011. 1-(6-(4-isopropyl-4H-1,2,4-triazol-3-yl)pyridin-2-yl)-3-(3-(pyridin-4-yl)phenyl)imidazolidin-2-one;
1012. 1-(6-(4-isopropyl-4H-1,2,4-triazol-3-yl)pyridin-2-yl)-3-(3-(1-methyl-1H-pyrazol-4-yl)phenyl)imidazolidin-2-one;
1013. 1-(6-(4-isopropyl-4H-1,2,4-triazol-3-yl)pyridin-2-yl)-3-(4-(methylsulfonyl)phenyl)imidazolidin-2-one;
1014. 1-(6-(4-isopropyl-4H-1,2,4-triazol-3-yl)pyridin-2-yl)-3-(3-(1-(methylsulfonyl)-1H-pyrazol-4-yl)phenyl)imidazolidin-2-one;
1015. 3′-(3-(6-(4-isopropyl-4H-1,2,4-triazol-3-yl)pyridin-2-yl)-2-oxoimidazolidin-1-yl)-[1,1′-biphenyl]-4-carboxamide;
1016. 3′-(3-(6-(4-isopropyl-4H-1,2,4-triazol-3-yl)pyridin-2-yl)-2-oxoimidazolidin-1-yl)-[1,1′-biphenyl]-4-carbonitrile;
1017. 1-(6-(4-isopropyl-4H-1,2,4-triazol-3-yl)pyridin-2-yl)-3-(4′-(trifluoromethyl)-[1,1′-biphenyl]-3-yl)imidazolidin-2-one;
1018. 1-(6-(4-isopropyl-4H-1,2,4-triazol-3-yl)pyridin-2-yl)-3-(6-(methylsulfonyl)pyridin-3-yl)imidazolidin-2-one;
1019. 1-(6-(4-isopropyl-4H-1,2,4-triazol-3-yl)pyridin-2-yl)-3-(2-methoxy-5-(6-(methylsulfonyl)pyridin-3-yl)phenyl)imidazolidin-2-one;
1020. 4-(3-(6-(4-isopropyl-4H-1,2,4-triazol-3-yl)pyridin-2-yl)-2-oxoimidazolidin-1-yl)benzenesulfonamide;
1021. 1-(2-fluoro-5-(6-(methylsulfonyl)pyridin-3-yl)phenyl)-3-(6-(4-isopropyl-4H-1,2,4-triazol-3-yl)pyridin-2-yl)imidazolidin-2-one;
1022. 1-(6-(4-isopropyl-4H-1,2,4-triazol-3-yl)pyridin-2-yl)imidazolidin-2-one;
1023. 4-(3-(6-(4-isopropyl-4H-1,2,4-triazol-3-yl)pyridin-2-yl)-2-oxoimidazolidin-1-yl)benzenesulfonamide;
1024. 1-(6-(4-isopropyl-4H-1,2,4-triazol-3-yl)pyridin-2-yl)-3-phenylimidazolidin-2-one;
1025. 1-(4-(cyclopropylsulfonyl)phenyl)-3-(6-(4-isopropyl-4H-1,2,4-triazol-3-yl)pyridin-2-yl)imidazolidin-2-one;
1026. 1-(6-(4-isopropyl-4H-1,2,4-triazol-3-yl)pyridin-2-yl)-3-(4-methoxy-6-methyl-4′-(methylsulfonyl)-[1,1′-biphenyl]-3-yl)imidazolidin-2-one;
1027. 1-(6-(2-methoxyethylsulfonyl)pyridin-3-yl)-3-(6-(4-isopropyl-4H-1,2,4-triazol-3-yl)pyridin-2-yl)imidazolidin-2-one;
1028. 1-(6-(4-isopropyl-4H-1,2,4-triazol-3-yl)pyridin-2-yl)-3-(methylsulfonyl)imidazolidin-2-one;
1029. 1-(6-(4-isopropyl-4H-1,2,4-triazol-3-yl)pyridin-2-yl)-3-(3-methyl-4-(methylsulfonyl)phenyl)imidazolidin-2-one;
1030. 1-(4-hydroxyphenyl)-3-(6-(4-isopropyl-4H-1,2,4-triazol-3-yl)pyridin-2-yl)imidazolidin-2-one 1031. 1-(6-(4-isopropyl-4H-1,2,4-triazol-3-yl)pyridin-2-yl)-3-(2-methoxy-4-methyl-5-(methylsulfonyl)phenyl)imidazolidin-2-one;
1032. 1-(2-fluoro-4-(methylsulfonyl)phenyl)-3-(6-(4-isopropyl-4H-1,2,4-triazol-3-yl)pyridin-2-yl)imidazolidin-2-one;
1033. 1-(4-(trifluoromethyl)phenyl)-3-(6-(4-isopropyl-4H-1,2,4-triazol-3-yl)pyridin-2-yl)imidazolidin-2-one;
1034. 1-(6-(4-isopropyl-4H-1,2,4-triazol-3-yl)pyridin-2-yl)-3-(pyridin-4-yl)imidazolidin-2-one;
1035. 4-(3-(6-(4-isopropyl-4H-1,2,4-triazol-3-yl)pyridin-2-yl)-2-oxoimidazolidin-1-yl)benzonitrile;
1036. 1-(4-(1H-imidazol-1-yl)phenyl)-3-(6-(4-isopropyl-4H-1,2,4-triazol-3-yl)pyridin-2-yl)imidazolidin-2-one;
1037. 1-(4-(cyclopropanecarbonyl)phenyl)-3-(6-(4-isopropyl-4H-1,2,4-triazol-3-yl)pyridin-2-yl)imidazolidin-2-one;
1038. 1-(3-fluoro-4-(methylsulfonyl)phenyl)-3-(6-(4-isopropyl-4H-1,2,4-triazol-3-yl)pyridin-2-yl)imidazolidin-2-one;
1039. 1-(6-(4-isopropyl-4H-1,2,4-triazol-3-yl)pyridin-2-yl)-3-(4-methoxyphenyl)imidazolidin-2-one;
1040. 1-(4-(4-cyclopropyl-1H-imidazol-1-yl)phenyl)-3-(6-(4-isopropyl-4H-1,2,4-triazol-3-yl)pyridin-2-yl)imidazolidin-2-one;
1041. 1-(4-(dimethylamino)phenyl)-3-(6-(4-isopropyl-4H-1,2,4-triazol-3-yl)pyridin-2-yl)imidazolidin-2-one;
1042. 1-(6-(4-isopropyl-4H-1,2,4-triazol-3-yl)pyridin-2-yl)-3-(2-methyl-4-(methylsulfonyl)phenyl)imidazolidin-2-one;
1043. 1-(6-(4-isopropyl-4H-1,2,4-triazol-3-yl)pyridin-2-yl)-3-(4-morpholinophenyl)imidazolidin-2-one;
1044. N-(4-(3-(6-(4-isopropyl-4H-1,2,4-triazol-3-yl)pyridin-2-yl)-2-oxoimidazolidin-1-yl)phenyl)methanesulfonamide;
1045. 4-(3-(6-(4-isopropyl-4H-1,2,4-triazol-3-yl)pyridin-2-yl)-2-oxoimidazolidin-1-yl)-N-methylbenzenesulfonamide;
1046. N-cyclopropyl-6-(3-(6-(4-isopropyl-4H-1,2,4-triazol-3-yl)pyridin-2-yl)-2-oxoimidazolidin-1-yl)benzo[d]thiazol-2-aminium 2,2,2-trifluoroacetate;
1047. 1-(6-(4-isopropyl-4H-1,2,4-triazol-3-yl)pyridin-2-yl)-3-(4-((methylsulfonyl)methyl)phenyl)imidazolidin-2-one;
1048. N-cyclopropyl-4-(3-(6-(4-isopropyl-4H-1,2,4-triazol-3-yl)pyridin-2-yl)-2-oxoimidazolidin-1-yl)benzamide;
1049. 1-(6-(4-cyclopropyl-4H-1,2,4-triazol-3-yl)pyridin-2-yl)-3-(4-(methylsulfonyl)phenyl)imidazolidin-2-one;
1050. 1-(4-(2-methoxyethylsulfonyl)phenyl)-3-(6-(4-isopropyl-4H-1,2,4-triazol-3-yl)pyridin-2-yl)imidazolidin-2-one;
1051. 1-(4-(cyclopropyl(hydroxy)methyl)phenyl)-3-(6-(4-isopropyl-4H-1,2,4-triazol-3-yl)pyridin-2-yl)imidazolidin-2-one;
1052. 2-(4-(3-(6-(4-isopropyl-4H-1,2,4-triazol-3-yl)pyridin-2-yl)-2-oxoimidazolidin-1-yl)phenylamino)-2-cyclopropylacetamide;
1053. 3-(3-(6-(3-(4-(methylsulfonyl)phenyl)-2-oxoimidazolidin-1-yl)pyridin-2-yl)-4H-1,2,4-triazol-4-yl)butanenitrile;
1054. 1-(6-(4-isopropyl-4H-1,2,4-triazol-3-yl)pyridin-2-yl)-3-(3-methoxy-4-(methylsulfonyl)phenyl)imidazolidin-2-one;
1055. 1-(6-(4-isopropyl-4H-1,2,4-triazol-3-yl)pyridin-2-yl)-3-(2-methoxy-5-methyl-4-(methylsulfonyl)phenyl)imidazolidin-2-one;
1056. 2-(4-(3-(6-(4-isopropyl-4H-1,2,4-triazol-3-yl)pyridin-2-yl)-2-oxoimidazolidin-1-yl)phenylamino)-2-cyclopropylacetonitrile;
1057. 1-(6-(4-(1-hydroxypropan-2-yl)-4H-1,2,4-triazol-3-yl)pyridin-2-yl)-3-(4-(methylsulfonyl)phenyl)imidazolidin-2-one;
1058. 1-(6-(4-isopropyl-4H-1,2,4-triazol-3-yl)pyridin-2-yl)-3-(4-(methylsulfonyl)-3-(pyrrolidin-1-yl)phenyl)imidazolidin-2-one;
1059. 1-(3-cyclopropyl-4-(methylsulfonyl)phenyl)-3-(6-(4-isopropyl-4H-1,2,4-triazol-3-yl)pyridin-2-yl)imidazolidin-2-one;
1060. 1-(3-cyclopropyl-4-fluorophenyl)-3-(6-(4-isopropyl-4H-1,2,4-triazol-3-yl)pyridin-2-yl)imidazolidin-2-one;
1061. 1-(6-(4-(1,1,1-trifluoropropan-2-yl)-4H-1,2,4-triazol-3-yl)pyridin-2-yl)-3-(4-(methylsulfonyl)phenyl)imidazolidin-2-one;
1062. 1-(3-isopropyl-4-(methylsulfonyl)phenyl)-3-(6-(4-isopropyl-4H-1,2,4-triazol-3-yl)pyridin-2-yl)imidazolidin-2-one;
1063. 1-(6-(4-isopropyl-4H-1,2,4-triazol-3-yl)pyridin-2-yl)-3-(4-(methylsulfonyl)-3-(piperidin-1-yl)phenyl)imidazolidin-2-one;
1064. 1-(5-(4-isopropyl-4H-1,2,4-triazol-3-yl)pyridin-2-yl)-3-(4-(methylsulfonyl)phenyl)imidazolidin-2-one;
1065. 1-(6-(4-isopropyl-4H-1,2,4-triazol-3-yl)pyridin-2-yl)-3-(4-(methylsulfonyl)phenyl)imidazolidin-2-one;
1066. 1-(4-(tetrahydro-2H-pyran-4-yl)phenyl)-3-(6-(4-isopropyl-4H-1,2,4-triazol-3-yl)pyridin-2-yl)imidazolidin-2-one;
1067. 1-(4-(4-hydroxypiperidin-1-yl)phenyl)-3-(6-(4-isopropyl-4H-1,2,4-triazol-3-yl)pyridin-2-yl)imidazolidin-2-one;
1068. 4-(4-(3-(6-(4-isopropyl-4H-1,2,4-triazol-3-yl)pyridin-2-yl)-2-oxoimidazolidin-1-yl)phenyl)-5-methylmorpholin-3-one;
1069. 1-(6-(4-isopropyl-4H-1,2,4-triazol-3-yl)pyridin-2-yl)-3-(4-(3-methylmorpholino)phenyl)imidazolidin-2-one;
1070. 1-(6-(4-isopropyl-4H-1,2,4-triazol-3-yl)pyridin-2-yl)-3-(4-(4,4-dimethylpiperidin-1-yl)phenyl)imidazolidin-2-one;
1071. 1-(4-(3-(6-(4-isopropyl-4H-1,2,4-triazol-3-yl)pyridin-2-yl)-2-oxoimidazolidin-1-yl)phenyl)azetidine-3-carbonitrile;
1072. 1-(6-(4-hydroxypiperidin-1-yl)pyridin-3-yl)-3-(6-(4-isopropyl-4H-1,2,4-triazol-3-yl)pyridin-2-yl)imidazolidin-2-one;
1073. 1-(6-(4-isopropyl-4H-1,2,4-triazol-3-yl)pyridin-2-yl)-3-(4-(morpholinosulfonyl)phenyl)imidazolidin-2-one;
1074. 1-(4-(2-oxa-6-azaspiro[3.3]heptan-6-yl)phenyl)-3-(6-(4-isopropyl-4H-1,2,4-triazol-3-yl)pyridin-2-yl)imidazolidin-2-one;
1075. 1-(3-fluoro-4-morpholinophenyl)-3-(6-(4-isopropyl-4H-1,2,4-triazol-3-yl)pyridin-2-yl)imidazolidin-2-one;
1076. 4-(4-(3-(6-(4-isopropyl-4H-1,2,4-triazol-3-yl)pyridin-2-yl)-2-oxoimidazolidin-1-yl)phenyl)-6-methylmorpholin-3-one;
1077. 1-(6-(4-isopropyl-4H-1,2,4-triazol-3-yl)pyridin-2-yl)-3-(4-(morpholine-4-carbonyl)phenyl)imidazolidin-2-one;
1078. 1-(6-(4-isopropyl-4H-1,2,4-triazol-3-yl)pyridin-2-yl)-3-(4-(2-methylmorpholino)phenyl)imidazolidin-2-one;
1079. 1-(6-(4-isopropyl-4H-1,2,4-triazol-3-yl)pyridin-2-yl)-3-(3-methyl-4-morpholinophenyl)imidazolidin-2-one;
1080. 1-(6-(4-isopropyl-4H-1,2,4-triazol-3-yl)pyridin-2-yl)-3-(4-(morpholinomethyl)phenyl)imidazolidin-2-one;
1081. 1-(6-(4-isopropyl-4H-1,2,4-triazol-3-yl)pyridin-2-yl)-3-(4-(tetrahydro-2H-pyran-4-ylamino)phenyl)imidazolidin-2-one;
1082. 1-(4-(3-(6-(4-isopropyl-4H-1,2,4-triazol-3-yl)pyridin-2-yl)-2-oxoimidazolidin-1-yl)phenyl)-N-methylpiperidine-4-carboxamide;
1083. 1-(4-(2,2,2-trifluoro-1-hydroxyethyl)phenyl)-3-(6-(4-isopropyl-4H-1,2,4-triazol-3-yl)pyridin-2-yl)imidazolidin-2-one;
1084. 1-(6-(4-isopropyl-4H-1,2,4-triazol-3-yl)pyridin-2-yl)-3-(4-(1-(methylsulfonyl)piperidin-4-yl)phenyl)imidazolidin-2-one;
1085. 1-(6-(4-isopropyl-4H-1,2,4-triazol-3-yl)pyridin-2-yl)-3-(4-((3R,5S)-3,5-dimethylmorpholino)phenyl)imidazolidin-2-one;
1086. 1-(6-(4-isopropyl-4H-1,2,4-triazol-3-yl)pyridin-2-yl)-3-(4-((3R,5R)-3,5-dimethylmorpholino)phenyl)imidazolidin-2-one;
1087. 1-(4-(3-(6-(4-isopropyl-4H-1,2,4-triazol-3-yl)pyridin-2-yl)-2-oxoimidazolidin-1-yl)phenyl)piperidine-4-carbonitrile;
1088. 1-(6-(4-isopropyl-4H-1,2,4-triazol-3-yl)pyridin-2-yl)-3-(4-(3-(isopropylamino)azetidin-1-yl)phenyl)imidazolidin-2-one;
1089. 1-(4-(3-hydroxy-3-isopropylazetidin-1-yl)phenyl)-3-(6-(4-isopropyl-4H-1,2,4-triazol-3-yl)pyridin-2-yl)imidazolidin-2-one;
1090. 1-(4-(3-hydroxyazetidin-1-yl)phenyl)-3-(6-(4-isopropyl-4H-1,2,4-triazol-3-yl)pyridin-2-yl)imidazolidin-2-one;
1091. 1-(4-(3-(6-(4-isopropyl-4H-1,2,4-triazol-3-yl)pyridin-2-yl)-2-oxoimidazolidin-1-yl)phenyl)pyrrolidine-3-carbonitrile;
1092. 1-(6-(4-isopropyl-4H-1,2,4-triazol-3-yl)pyridin-2-yl)-3-(4-(4-methylpiperazin-1-yl)phenyl)imidazolidin-2-one;
1093. 1-(4-(3-(6-(4-isopropyl-4H-1,2,4-triazol-3-yl)pyridin-2-yl)-2-oxoimidazolidin-1-yl)phenyl)azetidine-3-carboxamide;
1094. 1-(4-(3-(cyclopropylamino)azetidin-1-yl)phenyl)-3-(6-(4-isopropyl-4H-1,2,4-triazol-3-yl)pyridin-2-yl)imidazolidin-2-one;
1095. 1-(4-(3-(6-(4-isopropyl-4H-1,2,4-triazol-3-yl)pyridin-2-yl)-2-oxoimidazolidin-1-yl)benzoyl)azetidine-3-carbonitrile;
1096. 1-(4-(3-fluoroazetidin-1-yl)phenyl)-3-(6-(4-isopropyl-4H-1,2,4-triazol-3-yl)pyridin-2-yl)imidazolidin-2-one;
1097. 1-(4-(3-(6-(4-isopropyl-4H-1,2,4-triazol-3-yl)pyridin-2-yl)-2-oxoimidazolidin-1-yl)phenyl)-N,N-dimethylpiperidine-4-carboxamide;
1098. 2-(4-(3-(6-(4-isopropyl-4H-1,2,4-triazol-3-yl)pyridin-2-yl)-2-oxoimidazolidin-1-yl)phenyl)-2-morpholinoacetonitrile;
1099. 1-(6-(4-isopropyl-4H-1,2,4-triazol-3-yl)pyridin-2-yl)-3-methylimidazolidin-2-one;
1100. 1-(6-(4-isopropyl-4H-1,2,4-triazol-3-yl)pyridin-2-yl)-3-(4-(pyrrolidin-1-yl)phenyl)imidazolidin-2-one;
1101. 1-(6-(4-isopropyl-4H-1,2,4-triazol-3-yl)pyridin-2-yl)-3-(6-morpholinopyridin-3-yl)imidazolidin-2-one;
1102. 1-(6-(4-isopropyl-4H-1,2,4-triazol-3-yl)pyridin-2-yl)-3-(2-(methylsulfonyl)pyrimidin-5-yl)imidazolidin-2-one;
1103. 1-(6-(4-isopropyl-4H-1,2,4-triazol-3-yl)pyridin-2-yl)-3-(2-morpholinopyrimidin-5-yl)imidazolidin-2-one;
1104. 1-(6-(1H-imidazol-1-yl)pyridin-3-yl)-3-(6-(4-isopropyl-4H-1,2,4-triazol-3-yl)pyridin-2-yl)imidazolidin-2-one;
1105. 1-(6-(dimethylamino)pyridin-3-yl)-3-(6-(4-isopropyl-4H-1,2,4-triazol-3-yl)pyridin-2-yl)imidazolidin-2-one;
1106. 5-(3-(6-(4-isopropyl-4H-1,2,4-triazol-3-yl)pyridin-2-yl)-2-oxoimidazolidin-1-yl)-N-methylpyridine-2-carboxamide;
1107. 1-(5-(3-(6-(4-isopropyl-4H-1,2,4-triazol-3-yl)pyridin-2-yl)-2-oxoimidazolidin-1-yl)pyridin-2-yl)azetidine-3-carbonitrile;
1108. 1-(6-(4-isopropyl-4H-1,2,4-triazol-3-yl)pyridin-2-yl)-3-(3-morpholinophenyl)imidazolidin-2-one;
1109. N-cyclopropyl-5-(3-(6-(4-isopropyl-4H-1,2,4-triazol-3-yl)pyridin-2-yl)-2-oxoimidazolidin-1-yl)pyridine-2-carboxamide;
1110. N-isopropyl-5-(3-(6-(4-isopropyl-4H-1,2,4-triazol-3-yl)pyridin-2-yl)-2-oxoimidazolidin-1-yl)pyridine-2-carboxamide;
1111. 1-(6-(4-isopropyl-4H-1,2,4-triazol-3-yl)pyridin-2-yl)-3-(1-(4-morpholinophenyl)ethyl)imidazolidin-2-one;
1112. 1-(4-morpholinobenzyl)-3-(6-(4-isopropyl-4H-1,2,4-triazol-3-yl)pyridin-2-yl)imidazolidin-2-one;
1113. 2-(5-(3-(6-(4-isopropyl-4H-1,2,4-triazol-3-yl)pyridin-2-yl)-2-oxoimidazolidin-1-yl)pyridin-2-yl)-2-morpholinoacetonitrile;
1114. 1-(6-(4-isopropyl-4H-1,2,4-triazol-3-yl)pyridin-2-yl)-3-(4-(1-morpholinoethyl)phenyl)imidazolidin-2-one;
1115. 1-(5-(3-(6-(4-isopropyl-4H-1,2,4-triazol-3-yl)pyridin-2-yl)-2-oxoimidazolidin-1-yl)pyridin-2-yl)azetidine-3-carboxamide;
1116. 1-(5-(3-(6-(4-isopropyl-4H-1,2,4-triazol-3-yl)pyridin-2-yl)-2-oxoimidazolidin-1-yl)pyridin-2-yl)-N-methylpiperidine-4-carboxamide;
1117. 5-(3-(6-(4-isopropyl-4H-1,2,4-triazol-3-yl)pyridin-2-yl)-2-oxoimidazolidin-1-yl)-N-(3-methoxypropyl)pyridine-2-carboxamide;
1118. 1-(6-(3-methoxypropylamino)pyridin-3-yl)-3-(6-(4-isopropyl-4H-1,2,4-triazol-3-yl)pyridin-2-yl)imidazolidin-2-one;
1119. 1-(5-(3-(6-(4-isopropyl-4H-1,2,4-triazol-3-yl)pyridin-2-yl)-2-oxoimidazolidin-1-yl)pyridin-2-yl)-N-methylpyrrolidine-3-carboxamide;
1120. 1-(6-(4-isopropyl-4H-1,2,4-triazol-3-yl)pyridin-2-yl)-3-(6-(4-(methylsulfonyl)piperidin-1-yl)pyridin-3-yl)imidazolidin-2-one;
1121. 1-(6-(4-(dimethylamino)piperidin-1-yl)pyridin-3-yl)-3-(6-(4-isopropyl-4H-1,2,4-triazol-3-yl)pyridin-2-yl)imidazolidin-2-one;
1122. 1-(5-(3-(6-(4-isopropyl-4H-1,2,4-triazol-3-yl)pyridin-2-yl)-2-oxoimidazolidin-1-yl)pyridin-2-yl)-N-methylazetidine-3-carboxamide;
1123. 1-(6-(4-isopropyl-4H-1,2,4-triazol-3-yl)pyridin-2-yl)-3-(5-(methylsulfonyl)thiophen-2-yl)imidazolidin-2-one;
1124. 1-(3,3-dimethyl-1,1-dioxido-2,3-dihydrobenzo[b]thiophen-5-yl)-3-(6-(4-isopropyl-4H-1,2,4-triazol-3-yl)pyridin-2-yl)imidazolidin-2-one;
1125. 1-(6-(4-isopropyl-4H-1,2,4-triazol-3-yl)pyridin-2-yl)-3-(1-methyl-1H-indo1-5-yl)imidazolidin-2-one;
1126. 1-(4-(2-(methylsulfonyl)ethoxy)phenyl)-3-(6-(4-isopropyl-4H-1,2,4-triazol-3-yl)pyridin-2-yl)imidazolidin-2-one;
1127. 1-(1-(4-(3-(6-(4-isopropyl-4H-1,2,4-triazol-3-yl)pyridin-2-yl)-2-oxoimidazolidin-1-yl)phenyl)ethyl)-N-methylpiperidine-4-carboxamide;
1128. 1-(4-(3-(6-(4-isopropyl-4H-1,2,4-triazol-3-yl)pyridin-2-yl)-2-oxoimidazolidin-1-yl)benzyl)-N-methylpiperidine-4-carboxamide;
1129. 1-(6-(4-isopropyl-4H-1,2,4-triazol-3-yl)pyridin-2-yl)-3-(4-(2-methyl-1-morpholinopropyl)phenyl)imidazolidin-2-one;
1130. (R)-1-(4-(3-(6-(4-isopropyl-4H-1,2,4-triazol-3-yl)pyridin-2-yl)-2-oxoimidazolidin-1-yl)phenyl)-N-methylpyrrolidine-3-carboxamide;
1131. (S)-1-(4-(3-(6-(4-isopropyl-4H-1,2,4-triazol-3-yl)pyridin-2-yl)-2-oxoimidazolidin-1-yl)phenyl)-N-methylpyrrolidine-3-carboxamide;
1132. 1-(6-(4-isopropyl-4H-1,2,4-triazol-3-yl)pyridin-2-yl)-3-(2-morpholinopyridin-4-yl)imidazolidin-2-one;
1133. 1-(6-(4-isopropyl-4H-1,2,4-triazol-3-yl)pyridin-2-yl)-3-(4-(tetrahydrofuran-3-ylamino)phenyl)imidazolidin-2-one;
1134. 2-(1-(4-(3-(6-(4-isopropyl-4H-1,2,4-triazol-3-yl)pyridin-2-yl)-2-oxoimidazolidin-1-yl)phenyl)azetidin-3-yl)-3-methylbutanenitrile;
1135. 4-(4-(3-(6-(4-isopropyl-4H-1,2,4-triazol-3-yl)pyridin-2-yl)-2-oxoimidazolidin-1-yl)phenyl)-1-methylpiperazin-2-one;
1136. 4-(4-(3-(6-(4-isopropyl-4H-1,2,4-triazol-3-yl)pyridin-2-yl)-2-oxoimidazolidin-1-yl)phenyl)-N-methylpiperazine-1-carboxamide;
1137. 3-(4-(4-(3-(6-(4-isopropyl-4H-1,2,4-triazol-3-yl)pyridin-2-yl)-2-oxoimidazolidin-1-yl)phenyl)piperazin-1-yl)-3-oxopropanenitrile;
1138. 1-(4-(4-fluoropiperidin-1-yl)phenyl)-3-(6-(4-isopropyl-4H-1,2,4-triazol-3-yl)pyridin-2-yl)imidazolidin-2-one;
1139. 1-(6-(4-isopropyl-4H-1,2,4-triazol-3-yl)pyridin-2-yl)-3-(4-(4-(3-(methylamino)oxetan-3-yl)piperidin-1-yl)phenyl)imidazolidin-2-one;
1140. 8-(4-(3-(6-(4-isopropyl-4H-1,2,4-triazol-3-yl)pyridin-2-yl)-2-oxoimidazolidin-1-yl)phenyl)-N-methyl-8-aza-bicyclo[3.2.1]octane-3-carboxamide.

3. 1-(6-(4-isopropyl-4H-1,2,4-triazol-3-yl)pyridin-2-yl)imidazolidin-4-one as claimed in claim 1, their pharmaceutically acceptable salts and isomers of formula II: 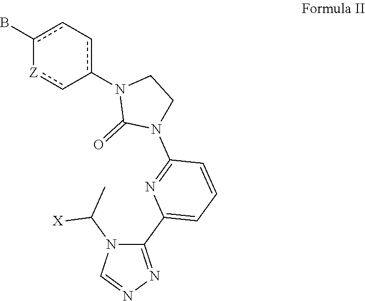
Wherein;
indicates an optional bond
B is absent, H, SO₂, SO₂(CH₂)n; SO₂R₁, SO₂(CH₂)nOR₁, SO₂NHR₁, NH₂(CH₂)n, CO NH₂(CH₂)n, NH₂(CH₂)n, NH₂(CH₂)nR₁CONH₂, NH₂(CH₂)nCN, NHCHR₁CN, NHR₁, SO₂Me, 3 to 8 carbocyclic ring optionally substituted by R1 or R2 or R3, 3 to 8 membered heterocyclic ring containing 1 to 3 heteroatoms selected from the group comprising O, N, S which may be optionally substituted by R1 or R2 or R3;
X is H, CH₃, (CH₂)n, C₁-C₆ straight or branched or cyclic alkyl, CN, (CH₂)CN, OH, (CH₂)nOH, CF₃;
R1, R2 and R3 are independently selected from the group comprising H, halo, CN, CF₃, CH₃, hydroxyl, C1-C₆ alkyl, C₁ to C₆ alkoxy , C1-C6 alkoxyalkyl and C 1 -C6 cyanoalkyl, amino, SO₂, SO₂C₁-C₆ Alkyl, CH₂CF₃, —(CH₂)nCN, —CH₂OMe, —CH₂SO₂Me, NR₄R5, CONR₄R₅, C₁-C₆ straight or branched or cyclic alkyl, C₂-C₆ straight or branched alkenyl, C₂-C₆ straight or branched alkynyl, halo-C₁-C₆ alkyl, C₁-C₆ alkyloxy; C₁-C₆ alkylamino, heteroalkyl, C₃-C₈ cycloalkyl, C₃-C₈cycloalkenyl, C₃-C₈heterocycloalkyl, C₃-C₈heterocycloalkenyl, carbocycyl, aryl, and heteroaryl; oxidised heteroaryl, oxidise heterocyclyl, heteroaryl or heterocyclyl that may be optionally substituted with R4 and R5
R4 and R5 may be selected from H, C₁-C₆ alkyl and C₃-C₈ cycloalkyl, NH₂, NHC₁-C₆ alkyl
Z can be CH or N
n is 0 to 3.

4. The compounds of formula II, as claimed in claim 3, selected from the group comprising:
1043: 1-(6-(4-isopropyl-4H-1,2,4-triazol-3-yl)pyridin-2-yl)-3-(4-morpholinophenyl)imidazolidin-2-one;
1071: 1-(4-(3-(6-(4-isopropyl-4H-1,2,4-triazol-3-yl)pyridin-2-yl)-2-oxoimidazolidin-1-yl)phenyl)azetidine-3-carbonitrile;
1082: 1-(4-(3-(6-(4-isopropyl-4H-1,2,4-triazol-3-yl)pyridin-2-yl)-2-oxoimidazolidin-1-yl)phenyl)-N-methylpiperidine-4-carboxamide;
1097: 1-(4-(3-(6-(4-isopropyl-4H-1,2,4-triazol-3-yl)pyridin-2-yl)-2-oxoimidazolidin-1-yl)phenyl)-N,N-dimethylpiperidine-4-carboxamide.

5. A process for preparing the compounds as claimed in claim 1, comprising the steps of: 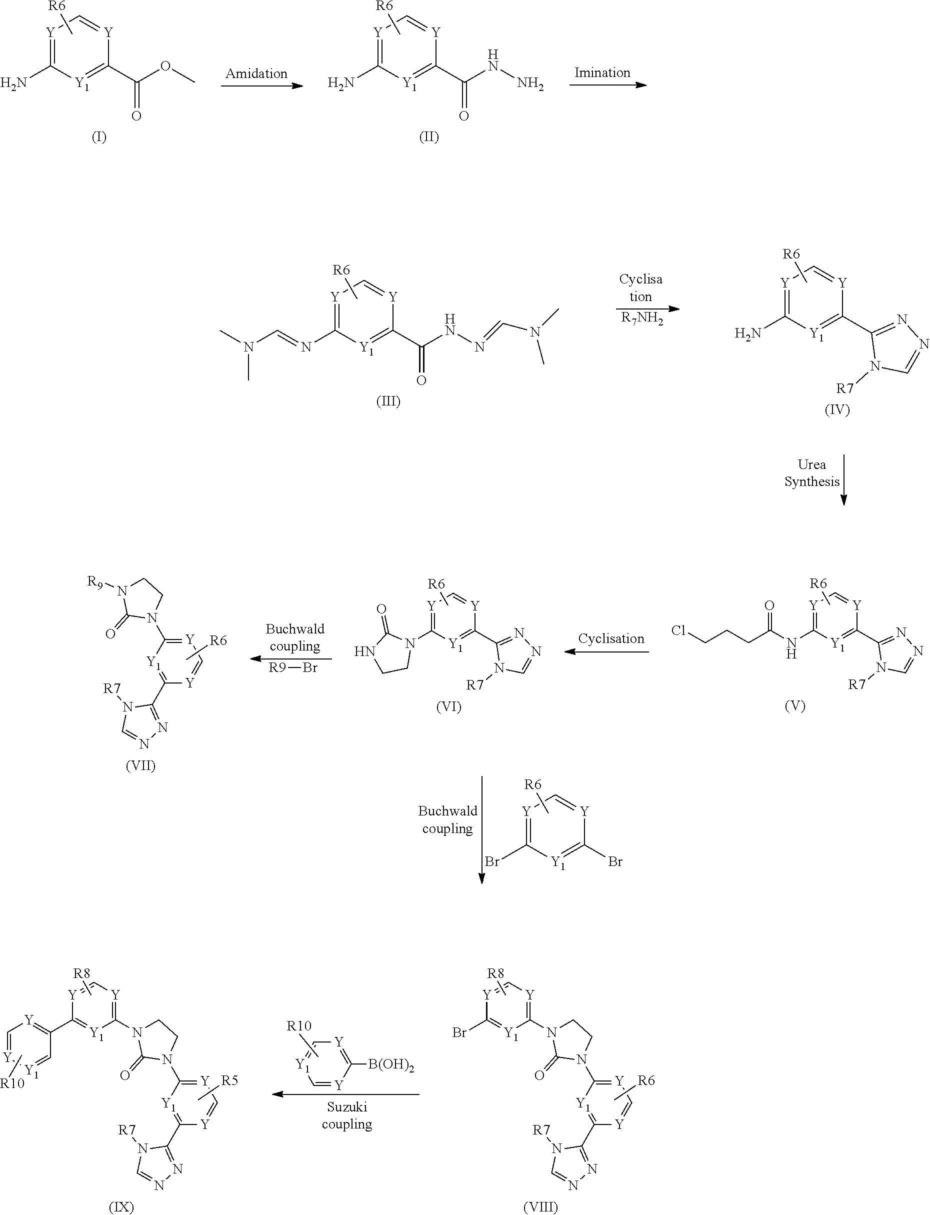
Wherein, Y and Y1 are CH or N;
Wherein, R₆=H, F, CH₃,
Wherein, R₁₀=—CN, —CF₃, —CONH₂, —SO₂CH₃, —CH₃,
Wherein, R₈=H, F, CH₃, OCH₃,
Wherein, 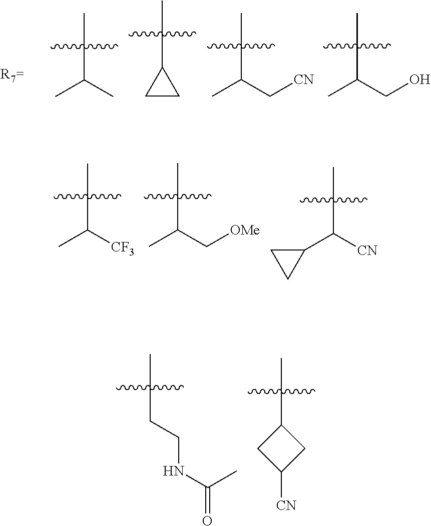
Wherein,

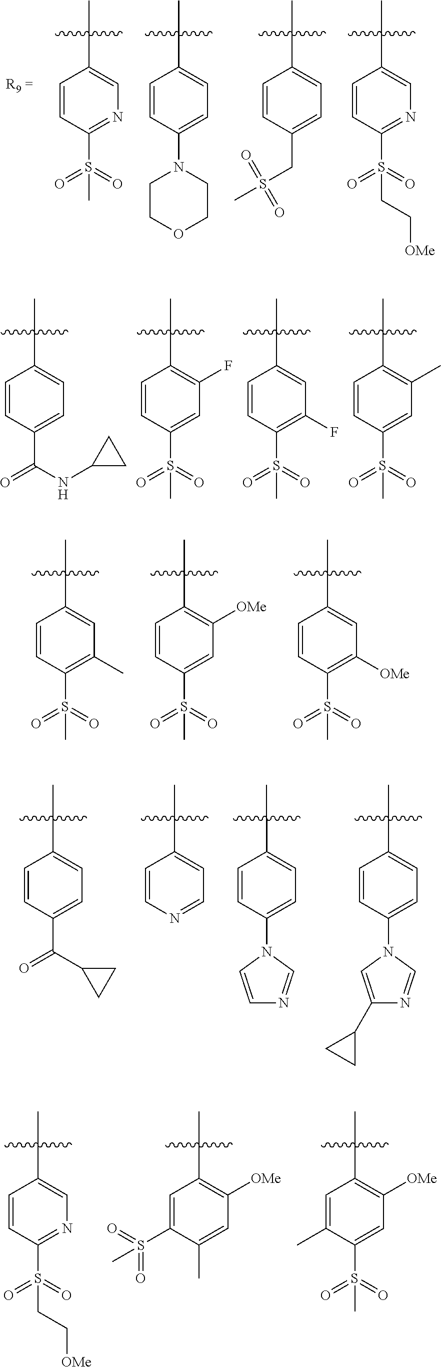

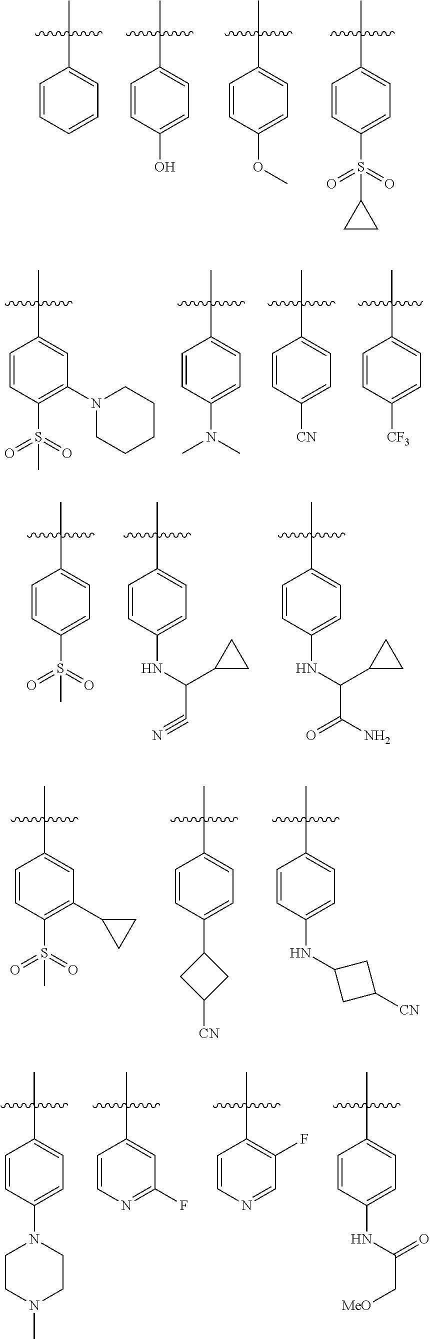

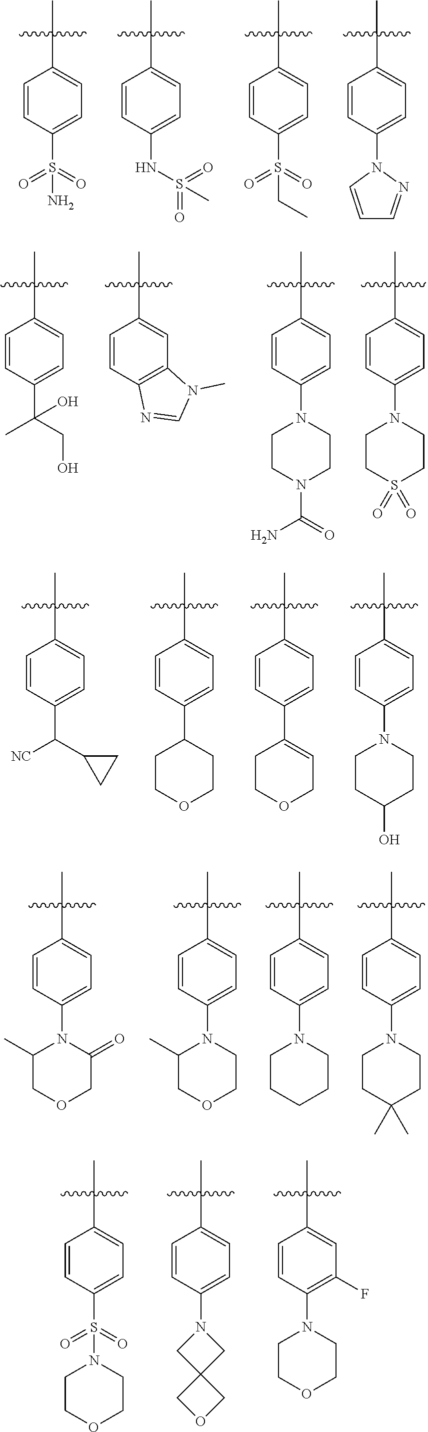

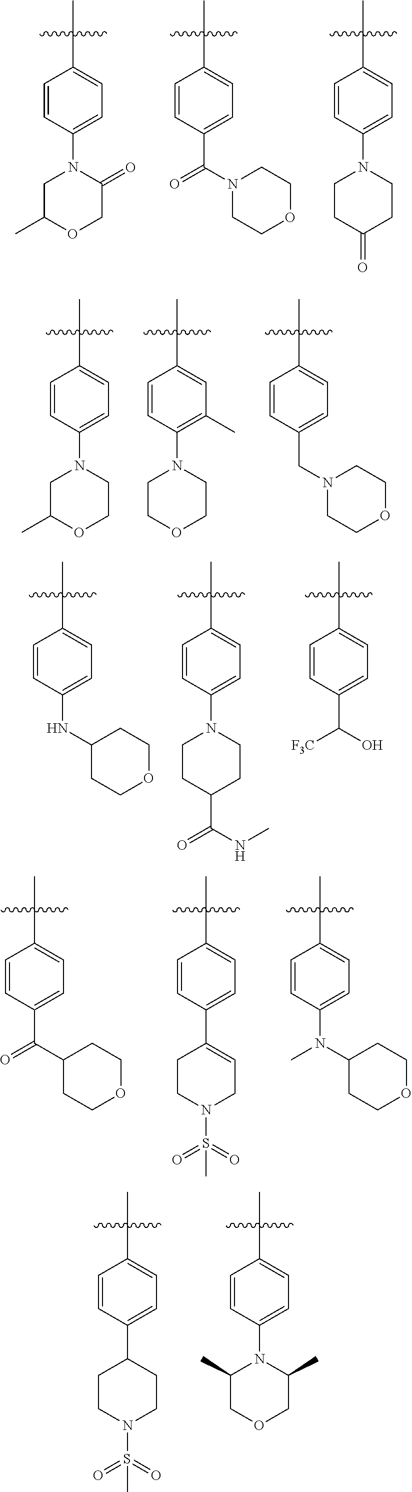

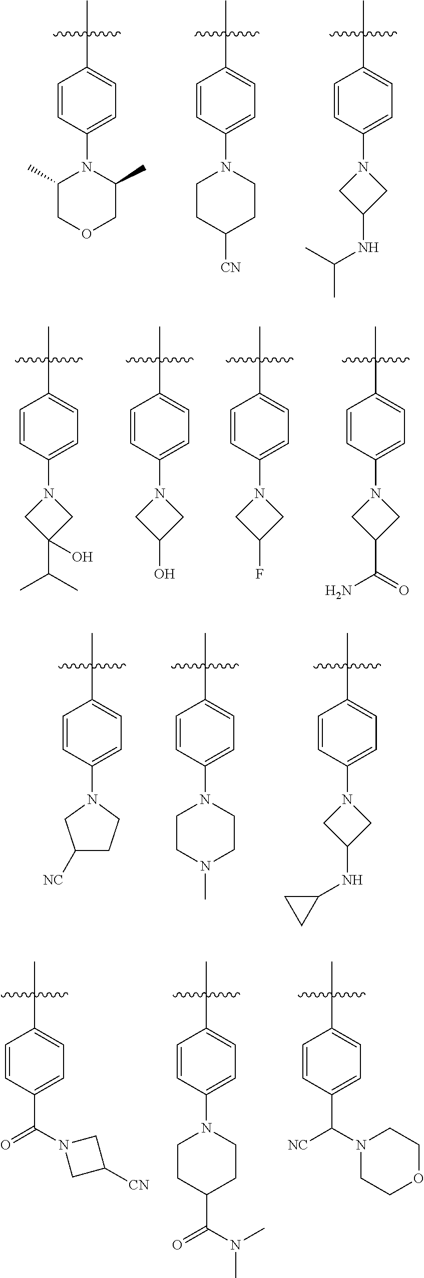

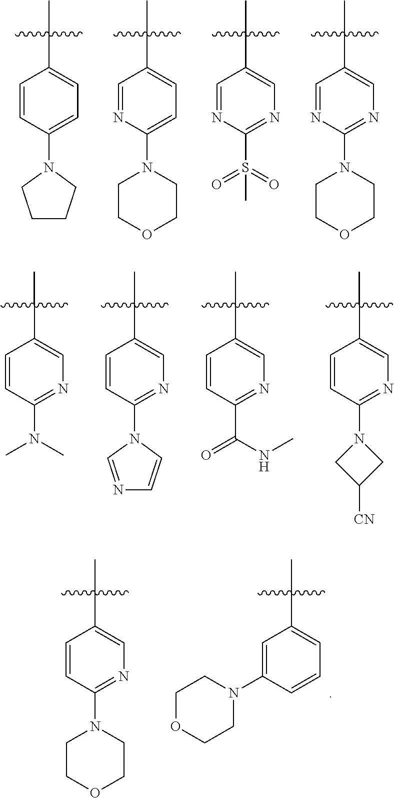

6. A Pharmaceutical composition comprising the compounds as claimed in claim 1 along with pharmaceutically acceptable excipients.

7. The Pharmaceutical composition as claimed in claim 6, when administered as orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms.

8. The compounds as claimed in claim 1, for their use in intervention of disorders pertaining to or related to inhibition or intervention of ASK1.

9. The compounds as claimed in claim 1, for their use in treating nonalcoholic fatty liver disease (NAFLD), obesity, metabolic syndrome, or any type of diabetes, including type 1 and type 2.

10. The compounds as claimed in claim 1, for their use in diabetic complications such as macro (ischemic heart disease, cerebrovascular disease and peripheral vascular disease) and micro (cataract, retinopathy, nephropathy, neuropathy, maculopathy and glaucoma) vascular complication; inflammation such as rheumatoid arthritis and cardiovascular diseases such as atherosclerosis, restenosis, hypertension, vasospasm, and cardiac hypertrophy; and lung disorders and lung fibrosis.
